(19) United States
(12) Patent Application Publication
(10) Pub. No.: US 2024/0132477 A1
(43) Pub. Date: Apr. 25, 2024

(54) Benzimidazole Derivatives and Their Use As Inhibitors of ITK For The Treatment of Skin Disease

(71) Applicant: PFIZER INC., New York, NY (US)

(72) Inventors: Scott William Bagley, Voluntown, CT (US); Agustin Casimiro Garcia, Concord, MA (US); Jennifer Elizabeth Davoren, Cambridge, MA (US); Rajiah Aldrin Denny, Sharon, MA (US); Brian Stephen Gerstenberger, Cambridge, MA (US); Frank Eldridge Lovering, Acton, MA (US); Mihir Dineshkumar Parikh, East Greenwich, RI (US); Joseph Walter Strohbach, Wentzville, MO (US); John Isidro Trujillo, Ledyard, CT (US)

(73) Assignee: Pfizer Inc., New York, NY (US)

(21) Appl. No.: 18/257,136

(22) PCT Filed: Dec. 13, 2021
(86) PCT No.: PCT/IB2021/061638

§ 371 Date: Jun. 13, 2023

Related U.S. Application Data
(60) Provisional application No. 63/125,650, filed on Dec. 15, 2020.

(51) Int. Cl. C07D 403/04; C07D 401/14; C07D 403/14; C07D 405/14; C07D 413/14

(52) U.S. Cl. CPC C07D 403/04; C07D 401/14; C07D 403/14; C07D 405/14; C07D 413/14

(57) ABSTRACT

The invention relates to benzimidazoles of Formula (I)

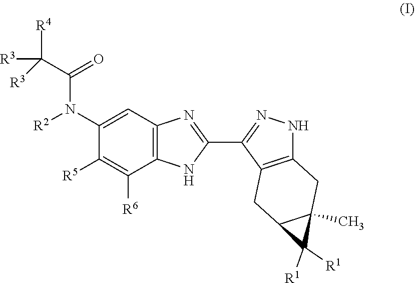

and pharmaceutically acceptable salts thereof, wherein R¹ to R⁶ are as defined in the description; to their use in medicine; to compositions containing them; to processes for their preparation; and to intermediates used in such processes.

The benzimidazoles of Formula (I) are ITK inhibitors and are therefore potentially useful in the treatment of a wide range of disorders including, atopic dermatitis.

The invention relates to benzimidazole derivatives, to their use in medicine, to compositions containing them, to processes for their preparation and to intermediates used in such processes. More especially the invention relates to inhibitors of interleukin-2-inducible T cell kinase (ITK) and their use in the treatment of diseases mediated by ITK, in particular skin diseases, such as dermatitis (e.g. atopic dermatitis).

Atopic dermatitis (AD) is a common chronic inflammatory skin disease with a prevalence in both children and adults. AD patients suffer from dry and pruritic skin lesions which can greatly affect their quality of life. Genetic and environmental factors can contribute to skin barrier disruption and immune hyper-activation which are key drivers of AD pathogenesis.

The pathogenic role for T cells and the Th2 cell-derived cytokines, IL-4 and IL-13, in AD has been shown through the clinical development of dupilumab, an antibody to the IL-4 receptor that blocks the activity of both IL-4 and IL-13. The important activity of these cytokines is also consistent with the early clinical efficacy that has been observed with Janus kinase (JAK) inhibitors, which block signaling of IL-4 and IL-13 as well as additional inflammatory cytokines produced in the skin. A therapeutic strategy that can effectively control the production of IL-4 and IL-13 is an alternative approach to modulate this pathway. Additionally, Th1 cells, Th22 cells, and Th17 cells and the cytokines which they produce, IFNγ, IL-22, and IL-17, respectively, also contribute to AD pathogenesis.

An effective anti-inflammatory for AD would modulate the predominant T cell driven inflammatory response. Interleukin-2-inducible T cell kinase (ITK) is a member of the Tec family of tyrosine kinases. ITK expression is largely limited to immune cells such as T, natural killer (NK), natural killer T (NKT), and mast cells. In T cells, ITK amplifies T cell receptor (TCR)-dependent signals to promote T cell activation, cytokine production, and T cell proliferation. ITK deletion or inhibition of ITK activity in T cells results in suppression of TCR-induced IL-4 and IL-13 production, which plays a central role in contributing to the pathophysiology of AD. An ITK inhibitor is expected to have additional efficacy compared to an antagonist of the IL-4 receptor, as ITK also contributes to TCR-dependent production of numerous pro-inflammatory cytokines such as IL-2, IL-17, IL-22, IL-31, IFNγ, and TNF-α. Additionally, ITK deficient CD8+ T cells demonstrate impaired cytotoxic T lymphocyte expansion, reduced degranulation and defective cytolytic capacity. ITK deficient mice and/or mice treated with an ITK inhibitor demonstrate reduced disease in models of type I diabetes, lymphoproliferative disease, allergy/asthma, and airway hyperresponsiveness. Moreover, ITK-deficient mice or mice treated with an ITK inhibitor demonstrate reduced skin inflammation in models of dermatitis. Elevated levels of ITK were described in peripheral T cells from patients with moderate to severe AD, and ITK expression is elevated in skin lesions from AD patients.

Additionally, tropomyosin receptor kinases (TRKs) are expressed by cells in the skin such as keratinocytes, neurons, mast cells, and basophils. Both TRKA and its ligand, nerve growth factor (NGF), are present in the skin and their expression is enhanced in AD skin lesions. Levels of NGF in skin lesions from AD patients have been demonstrated to correlate with itch severity. Cytokines IL-4 and IL-13 which contribute to AD pathogenesis have been demonstrated to enhance TRKA expression by keratinocytes. In addition to regulating development and maintenance of neurons, NGF can sensitize nociceptors and promote pruritis in the skin. Pruritis is a major factor contributing to reduced quality of life for AD patients. A therapy which can suppress pruritis would not only provide relief for patients, but may also break the itch-scratch cycle which contributes to the barrier disruption and thus reduce the course and chronicity of the disease.

NGF is also expressed by and has effects on non-neuronal cells. NGF induces keratinocyte proliferation, promotes basophil activation, stimulates mast cell degranulation, and contributes to neurogenic itch and inflammation. Furthermore, TRKA expression has been reported on TCR-stimulated peripheral blood T cells and T cells collected from synovial fluid from arthritis patients, and NGF induces proliferation of T cells. Thus, inhibiting TRKA in the skin may suppress dermal inflammation in addition to reducing pruritis.

These data suggest that an ITK inhibitor will suppress pathogenic T cell responses and reduce cytokine production, and therefore have therapeutic value in the treatment of a variety of inflammatory and autoimmune diseases, including dermatological conditions, such as atopic dermatitis, contact dermatitis, psoriasis, alopecia areata, and vitiligo.

Moreover an inhibitor of both ITK and TRKA activity should be of particular advantage in the treatment of dermatological conditions, such as those just mentioned (e.g. atopic dermatitis).

### REFERENCES

Benecke H, et al. Expert Opin. Invest. Drugs. 2013; 22:1167-1179;
Bissonette R, Papp K A, et al. Brit. J. Derm. 2016; 175:902-911;
Botchkarev V A, Yaar M, Peters E M J et al. J. Invest. Dermatology. 2006; 126:1719-1727;
Brunner P M, et al. J Allergy Clin Immunol. 2017;139(4S):S65-S76;
Kapnick S M, Stinchcombe J C, et al. Immunol. 2017; 198:2699-2711;
Lin T A, McIntyre K W, et al. Biochemistry 2004; 43:11056-11062;
Matsumura S, Terao M, et al. J. Derm. Science 2015; 78:215-223;
Otsuka A, Nomura T, et al. Immunological Reviews. 2017; 278:246-262;
Raychaudhuri S P, Raychaudhuri S K, et al. Arthritis & Rheumatism 2011; 63:3243-3252;
Sabat R, Wolk K, et al. Seminars in Immunopathology 2019; 41:359-377;
Sahu N, and August A. Curr. Top. Med. Chem. 2009; 9:690-703;
Von Bonin A, Rausch A, et al. Exp. Derm. 2010; 20:41-47;
Yamaguchi J, Aihara M, et al. J. Dermatol. Science. 2008; 53:48-54

According to a first aspect of the invention there is provided a compound of Formula (I)

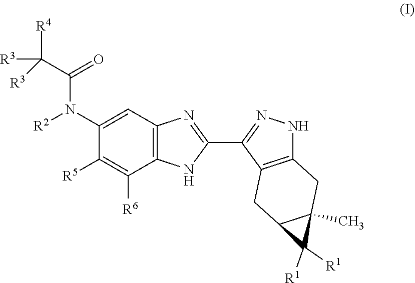

or a pharmaceutically acceptable salt thereof, wherein:
each R¹ is independently H or F;
R² is H or (C₁-C₄)alkyl;
each R³ is independently H, F or (C₁-C₄)alkyl; or
both R³ taken together with the carbon atom to which they are attached form:
   a (C₃-C₆)cycloalkyl, optionally substituted by one or two F;
   a (C₆-C₇)bicycloalkyl, optionally substituted by one or two F or
   a C-linked 4-7 membered saturated heterocycle containing one O;
R⁴ is H, F, (C₁-C₆)alkyl, (C₁-C₆)alkoxy, —NR⁷R⁸, or N-linked 4-8 membered saturated heterocycle containing one N and optionally one O (with the proviso that R⁴ is not morpholinyl) wherein said heterocycle is optionally substituted by oxo;
R⁵ is H, halogen, (C₁-C₆)alkyl, (C₁-C₆)alkoxy, or (C₁-C₆)alkoxy substituted by (C₁-C₄)alkoxy;
R⁶ is independently H or F;
R⁷ is (C₁-C₆)alkyl; and
R⁸ is (C₁-C₆)alkyl or —C(O)(C₁-C₆)alkyl.

Described below are embodiments of this first aspect of the invention, where for convenience E1 is identical thereto.
E1 A compound of Formula (I) or a pharmaceutically acceptable salt thereof as defined above.
E2 A compound according to embodiment E1 or a pharmaceutically acceptable salt thereof wherein each R¹ is H.
E3 A compound according to embodiment E1 or a pharmaceutically acceptable salt thereof wherein each R¹ is F.
E4 A compound according to any one of embodiments E1 to E3 or a pharmaceutically acceptable salt thereof wherein R² is H, methyl or ethyl.
E5 A compound according to embodiment E4 or a pharmaceutically acceptable salt thereof, wherein R² is methyl.
E6 A compound according to any one of embodiments E1 to E5 or a pharmaceutically acceptable salt thereof wherein each R³ is independently H, methyl or ethyl.
E7 A compound according to embodiment E6 or a pharmaceutically acceptable salt thereof wherein one R³ is H and the other R³ is methyl.
E8 A compound according to embodiment E6 or a pharmaceutically acceptable salt thereof wherein each R³ is methyl.
E9 A compound according to embodiment E6 or a pharmaceutically acceptable salt thereof wherein each R³ is H.
E10 A compound according to any one of embodiments E1 to E5 or a pharmaceutically acceptable salt thereof wherein both R³ taken together with the carbon atom to which they are attached form:
   a (C₃-C₆)cycloalkyl, optionally substituted by one or two F;
   a (C₆-C₇)bicycloalkyl, optionally substituted by one or two F; or
   a C-linked 4-7 membered saturated heterocycle containing one O.
E11 A compound according to embodiment E10 or a pharmaceutically acceptable salt thereof wherein both R³ taken together with the carbon atom to which they are attached form a (C₃-C₆)cycloalkyl.
E12 A compound according to embodiment E11 or a pharmaceutically acceptable salt thereof wherein both R³ taken together with the carbon atom to which they are attached form a (C₃-C₄)cycloalkyl.
E13 A compound according to embodiment E10 or a pharmaceutically acceptable salt thereof wherein both R³ taken together with the carbon atom to which they are attached form a (C₆-C₇)bicycloalkyl substituted by two F.
E14 A compound according to embodiment E10 or a pharmaceutically acceptable salt thereof wherein both R³ taken together with the carbon atom to which they are attached form a C-linked 5-6 membered saturated heterocycle containing one O.
E15 A compound according to any one of embodiments E1 to E14 or a pharmaceutically acceptable salt thereof wherein R⁴ is H, F, (C₁-C₃)alkyl, (C₁-C₃)alkoxy, —NR⁷R⁸, or N-linked 5-7 membered saturated heterocycle containing one N and optionally one O (with the proviso that R⁴ is not morpholinyl) wherein said heterocycle is optionally substituted by oxo.
E16 A compound according to embodiment E15 or a pharmaceutically acceptable salt thereof wherein R⁴ is H, F, (C₁-C₃)alkyl, (C₁-C₃)alkoxy or —NR⁷R⁸.
E17 A compound according to embodiment E16 or a pharmaceutically acceptable salt thereof wherein R⁴ is H, F or —NR⁷R⁸.
E18 A compound according to embodiment E17 or a pharmaceutically acceptable salt thereof wherein R⁴ is —NR⁷R⁸.
E19 A compound according to any one of embodiments E1 to E18 or a pharmaceutically acceptable salt thereof wherein R⁷ is (C₁-C₃)alkyl.
E20 A compound according to embodiment E19 or a pharmaceutically acceptable salt thereof wherein R⁷ is methyl.
E21 A compound according to any one of embodiments E1 to E20 or a pharmaceutically acceptable salt thereof wherein R⁸ is —C(O)(C₁-C₃)alkyl.
E22 A compound according to embodiment E21 or a pharmaceutically acceptable salt thereof wherein R⁸ is —C(O)CH3 or —C(O)CH(CH₃)₂.
E23 A compound according to embodiment E16 or a pharmaceutically acceptable salt thereof wherein R⁴ is H or F.
E24 A compound according to embodiment E23 or a pharmaceutically acceptable salt thereof wherein R⁴ is H.
E25 A compound according to embodiment E15 or a pharmaceutically acceptable salt thereof wherein R⁴ is N-linked 5-7 membered saturated heterocycle containing one N and optionally one O (with the proviso that R⁴ is not morpholinyl) wherein said heterocycle is optionally substituted by oxo.
E26 A compound according to embodiment E25 or a pharmaceutically acceptable salt thereof wherein R⁴ is N-linked 5-7 membered saturated heterocycle containing one N wherein said heterocycle is optionally substituted by oxo.
E27 A compound according to embodiment E26 or a pharmaceutically acceptable salt thereof wherein R⁴ is N-linked 5-7 membered saturated heterocycle containing one N wherein said heterocycle is substituted by oxo.
E28 A compound according to embodiment E27 or a pharmaceutically acceptable salt thereof wherein R⁴ is

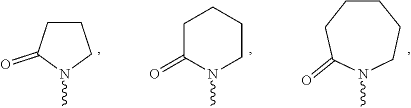

E29 A compound according to embodiment E25 or a pharmaceutically acceptable salt thereof wherein R⁴ is N-linked 5-7 membered saturated heterocycle containing one N and one O (with the proviso that R⁴ is not morpholinyl) wherein said heterocycle is optionally substituted by oxo.
E30 A compound according to embodiment E29 or a pharmaceutically acceptable salt thereof wherein R⁴ is N-linked 5-7 membered saturated heterocycle containing one N and one O (with the proviso that R⁴ is not morpholinyl) wherein said heterocycle is substituted by oxo.
E31 A compound according to embodiment E30 or a pharmaceutically acceptable salt thereof wherein R⁴ is

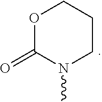

E32 A compound according to any one of embodiments E1 to E31 or a pharmaceutically acceptable salt thereof wherein R⁵ is H, halogen, (C₁-C₃)alkyl, (C₁-C₃)alkoxy, or (C₁-C₃)alkoxy substituted by (C₁-C₃)alkoxy.
E33 A compound according to embodiment E32 or a pharmaceutically acceptable salt thereof wherein R⁵ is H, methyl or ethyl.
E34 A compound according to embodiment E33 or a pharmaceutically acceptable salt thereof wherein R⁵ is methyl.
E35 A compound according to embodiment E33 or a pharmaceutically acceptable salt thereof wherein R⁵ is H.
E36 A compound according to any one of embodiments E1 to E35 or a pharmaceutically acceptable salt thereof wherein R⁶ is F.
E37 A compound according to any one of embodiments E1 to E35 or a pharmaceutically acceptable salt thereof wherein R⁶ is H.
E38 A compound according to embodiment E1 or a pharmaceutically acceptable salt thereof selected from:
   Example 1: N-Methyl-N-(6-methyl-2-((4aS,5aR)-5a-methyl-1,4,4a,5,5a,6-hexahydrocyclopropa[f]indazol-3-yl)-1H-benzo[d]imidazol-5-yl)-2-(2-oxo-1,3-oxazinan-3-yl)acetamide;
   Example 2: N-(2-((4aS,5aR)-5,5-difluoro-5a-methyl-1,4,4a,5,5a,6-hexahydrocyclopropa[f]indazol-3-yl)-1H-benzo[d]imidazol-5-yl)-N-methylacetamide;
   Example 3: N-(2-((4aR,5aS)-5,5-difluoro-5a-methyl-1,4,4a,5,5a,6-hexahydrocyclopropa[f]indazol-3-yl)-1H-benzo[d]imidazol-5-yl)-N-methylacetamide;
   Example 4: N-(6-methyl-2-((4aS,5aR)-5a-methyl-1,4,4a,5,5a,6-hexahydrocyclopropa[f]indazol-3-yl)-1H-benzo[d]imidazol-5-yl)acetamide;
   Example 5: N-ethyl-N-(6-methyl-2-((4aS,5aR)-5a-methyl-1,4,4a,5,5a,6-hexahydrocyclopropa[f]indazol-3-yl)-1H-benzo[d]imidazol-5-yl)acetamide;
   Example 6: N-(6-methoxy-2-((4aS,5aR)-5a-methyl-1,4,4a,5,5a,6-hexahydrocyclopropa[f]indazol-3-yl)-1H-benzo[d]imidazol-5-yl)-N-methylacetamide;
   Example 7: N-(6-(2-methoxyethoxy)-2-((4aS,5aR)-5a-methyl-1,4,4a,5,5a,6-hexahydrocyclopropa[f]indazol-3-yl)-1H-benzo[d]imidazol-5-yl)-N-methylacetamide;
   Example 8: N-(7-fluoro-6-methyl-2-((4aS,5aR)-5a-methyl-1,4,4a,5,5a,6-hexahydrocyclopropa[f]indazol-3-yl)-1H-benzo[d]imidazol-5-yl)-N-methylacetamide;
   Example 9: N-(6-bromo-2-((4aS,5aR)-5a-methyl-1,4,4a,5,5a,6-hexahydrocyclopropa[f]indazol-3-yl)-1H-benzo[d]imidazol-5-yl)-N-methylacetamide;
   Example 10: (R)—N-(4-fluoro-2-((4aS,5aR)-5a-methyl-1,4,4a,5,5a,6-hexahydrocyclopropa[f]indazol-3-yl)-1H-benzo[d]imidazol-6-yl)-N-methyl-2-(2-oxo-1,3-oxazinan-3-yl)propenamide;
   Example 11: (S)—N-(4-fluoro-2-((4aS,5aR)-5a-methyl-1,4,4a,5,5a,6-hexahydrocyclopropa[f]indazol-3-yl)-1H-benzo[d]imidazol-6-yl)-N-methyl-2-(2-oxo-1,3-oxazinan-3-yl)propenamide;
   Example 12: 4-fluoro-N-methyl-N-(5-methyl-2-((4aS,5aR)-5a-methyl-1,4,4a,5,5a,6-hexahydrocyclopropa[f]indazol-3-yl)-1H-benzo[d]imidazol-6-yl)tetrahydro-2H-pyran-4-carboxamide;
   Example 13: (1R,5S)-6,6-difluoro-N-methyl-N-(5-methyl-2-((4aS,5aR)-5a-methyl-1,4,4a,5,5a,6-hexahydrocyclopropa[f]indazol-3-yl)-1H-benzo[d]imidazol-6-yl)bicyclo[3.1.0]hexane-3-carboxamide;
   Example 14: N-methyl-N-(6-methyl-2-((4aS,5aR)-5a-methyl-1,4,4a,5,5a,6-hexahydrocyclopropa[f]indazol-3-yl)-1H-benzo[d]imidazol-5-yl)-2-(piperidin-1-yl)propenamide;
   Example 15: N-methyl-N-(6-methyl-2-((4aS,5aR)-5a-methyl-1,4,4a,5,5a,6-hexahydrocyclopropa[f]indazol-3-yl)-1H-benzo[d]imidazol-5-yl)-2-(1,4-oxazepan-4-yl)propenamide;
   Example 16: N-(6-ethyl-2-((4aS,5aR)-5a-methyl-1,4,4a,5,5a,6-hexahydrocyclopropa[f]indazol-3-yl)-1H-benzo[d]imidazol-5-yl)-N-methylacetamide;
   Example 17: N-Methyl-N-(6-methyl-2-((4aS,5aR)-5a-methyl-1,4,4a,5,5a,6-hexahydrocyclopropa[f]indazol-3-yl)-1H-benzo[d]imidazol-5-yl)acetamide;
   Example 18: N-Methyl-N-(5-methyl-2-((4aS,5aR)-5a-methyl-1,4,4a,5,5a,6-hexahydrocyclopropa[f]indazol-3-yl)-1H-benzo[d]imidazol-6-yl)-2-(2-oxopyrrolidin-1-yl)propenamide;
   Example 19: N-Methyl-N-(5-methyl-2-((4aS,5aR)-5a-methyl-1,4,4a,5,5a,6-hexahydrocyclopropa[f]indazol-3-yl)-1H-benzo[d]imidazol-6-yl)cyclopropanecarboxamide;
   Example 20: N-Methyl-N-(5-methyl-2-((4aS,5aR)-5a-methyl-1,4,4a,5,5a,6-hexahydrocyclopropa[f]indazol-3-yl)-1H-benzo[d]imidazol-6-yl)-2-(2-oxopiperidin-1-yl)acetamide;
      Example 21: N-Methyl-N-(5-methyl-2-((4aS,5aR)-5a-methyl-1,4,4a,5,5a,6-hexahydrocyclopropa[f]indazol-3-yl)-1H-benzo[d]imidazol-6-yl)-2-(N-methylacetamido)acetamide;
   Example 22: N-Methyl-N-(5-methyl-2-((4aS,5aR)-5a-methyl-1,4,4a,5,5a,6-hexahydrocyclopropa[f]indazol-3-yl)-1H-benzo[d]imidazol-6-yl)isobutyramide;
   Example 23: (R)—N-Methyl-N-(5-methyl-2-((4aS,5aR)-5a-methyl-1,4,4a,5,5a,6-hexahydrocyclopropa[f]indazol-3-yl)-1H-benzo[d]imidazol-6-yl)tetrahydrofuran-2-carboxamide;
   Example 24: N,1-Dimethyl-N-(5-methyl-2-((4aS,5aR)-5a-methyl-1,4,4a,5,5a,6-hexahydrocyclopropa[f]indazol-3-yl)-1H-benzo[d]imidazol-6-yl)cyclopropane-1-carboxamide;
   Example 25: (S)—N-Methyl-N-(6-methyl-2-((4aS,5aR)-5a-methyl-1,4,4a,5,5a,6-hexahydrocyclopropa[f]indazol-3-yl)-1H-benzo[d]imidazol-5-yl)-2-(2-oxopyrrolidin-1-yl)propenamide;
   Example 26: N-Methyl-N-(6-methyl-2-((4aS,5aR)-5a-methyl-1,4,4a,5,5a,6-hexahydrocyclopropa[f]indazol-3-yl)-1H-benzo[d]imidazol-5-yl)butyramide;
   Example 27: (S)—N-Methyl-N-(6-methyl-2-((4aS,5aR)-5a-methyl-1,4,4a,5,5a,6-hexahydrocyclopropa[f]indazol-3-yl)-1H-benzo[d]imidazol-5-yl)-2-(2-oxopyrrolidin-1-yl)butanamide;
   Example 28: 2-Fluoro-N,2-dimethyl-N-(6-methyl-2-((4aS,5aR)-5a-methyl-1,4,4a,5,5a,6-hexahydrocyclopropa[f]indazol-3-yl)-1H-benzo[d]imidazol-5-yl)propenamide;
   Example 29: (R)—N-Methyl-N-(6-methyl-2-((4aS,5aR)-5a-methyl-1,4,4a,5,5a,6-hexahydrocyclopropa[f]indazol-3-yl)-1H-benzo[d]imidazol-5-yl)tetrahydrofuran-3-carboxamide;
   Example 30: N-Methyl-N-(2-(methyl(6-methyl-2-((4aS,5aR)-5a-methyl-1,4,4a,5,5a,6-hexahydrocyclopropa[f]indazol-3-yl)-1H-benzo[d]imidazol-5-yl)amino)-2-oxoethyl)isobutyramide;
   Example 31: N-Methyl-N-(6-methyl-2-((4aS,5aR)-5a-methyl-1,4,4a,5,5a,6-hexahydrocyclopropa[f]indazol-3-yl)-1H-benzo[d]imidazol-5-yl)-2-(2-oxopyrrolidin-1-yl)acetamide;
   Example 32: 1-Fluoro-N-methyl-N-(6-methyl-2-((4aS,5aR)-5a-methyl-1,4,4a,5,5a,6-hexahydrocyclopropa[f]indazol-3-yl)-1H-benzo[d]imidazol-5-yl)cyclopropane-1-carboxamide;
   Example 33: N-Methyl-N-(6-methyl-2-((4aS,5aR)-5a-methyl-1,4,4a,5,5a,6-hexahydrocyclopropa[f]indazol-3-yl)-1H-benzo[d]imidazol-5-yl)propionamide;
   Example 34: N-Methyl-N-(6-methyl-2-((4aS,5aR)-5a-methyl-1,4,4a,5,5a,6-hexahydrocyclopropa[f]indazol-3-yl)-1H-benzo[d]imidazol-5-yl)-2-(2-oxoazepan-1-yl)acetamide;
   Example 35: 2-(N-Isopropylacetamido)-N-methyl-N-(6-methyl-2-((4aS,5aR)-5a-methyl-1,4,4a,5,5a,6-hexahydrocyclopropa[f]indazol-3-yl)-1H-benzo[d]imidazol-5-yl)acetamide;
   Example 36: N-Methyl-N-(6-methyl-2-((4aS,5aR)-5a-methyl-1,4,4a,5,5a,6-hexahydrocyclopropa[f]indazol-3-yl)-1H-benzo[d]imidazol-5-yl)cyclobutanecarboxamide;
   Example 37: N-Methyl-N-(6-methyl-2-((4aS,5aR)-5a-methyl-1,4,4a,5,5a,6-hexahydrocyclopropa[f]indazol-3-yl)-1H-benzo[d]imidazol-5-yl)-2-(2-oxopyrrolidin-1-yl)butanamide;
   Example 38: 2-Ethoxy-N-methyl-N-(6-methyl-2-((4aS,5aR)-5a-methyl-1,4,4a,5,5a,6-hexahydrocyclopropa[f]indazol-3-yl)-1H-benzo[d]imidazol-5-yl)acetamide;
   Example 39: 2-Methoxy-N,2-dimethyl-N-(6-methyl-2-((4aS,5aR)-5a-methyl-1,4,4a,5,5a,6-hexahydrocyclopropa[f]indazol-3-yl)-1H-benzo[d]imidazol-5-yl)propenamide; and
   Example 40: (S)—N-Methyl-N-(6-methyl-2-((4aS,5aR)-5a-methyl-1,4,4a,5,5a,6-hexahydrocyclopropa[f]indazol-3-yl)-1H-benzo[d]imidazol-5-yl)tetrahydrofuran-3-carboxamide.

In compounds of Formula (I):
Alkyl means a straight or branched chain hydrocarbon group of formula —CₙH₍₂ₙ₊₁₎. Examples of alkyl include methyl, ethyl, n-propyl, i-propyl, n-butyl, i-butyl, sec-butyl and t-butyl.
Alkyloxy means an alkyl substituent attached through an oxygen atom. Examples of alkyloxy include methoxy, ethoxy, n-propoxy, i-propoxy, n-butoxy, i-butoxy, sec-butoxy and t-butoxy.
Cycloalkyl means a cyclic hydrocarbon group of formula —CₙH₍₂ₙ₋₁₎ containing at least three carbon atoms. Examples of cycloalkyl include cyclopropyl, cyclobutyl, cyclopentyl and cyclohexyl.
Bicycloalkyl means a bicyclic hydrocarbon group in which the two rings are fused or form a bridged structure. An example of bicycloalkyl includes bicyclo[3.1.0]hexyl.
Oxo refers to a double bonded oxygen (═O).
Examples of halogen include fluoro (F), chloro (Cl), bromo (Br) and iodo (1).
Examples of a C-linked 4-7 membered saturated heterocycle containing one O include tetrahydrofuran-2-yl, tetrahydrofuran-3-yl and tetrahydro-2H-pyran-4-yl.

Hereinafter, all references to compounds of the invention include compounds of Formula (I) or pharmaceutically acceptable salts, solvates, or multi-component complexes thereof, or pharmaceutically acceptable solvates or multi-component complexes of pharmaceutically acceptable salts of compounds of Formula (I), as discussed in more detail below.

Preferred compounds of the invention are compounds of Formula (I) or pharmaceutically acceptable salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, 1,5-naphathalenedisulfonate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Hemisalts of acids may also be formed, for example, hemisulphate and hemitartrate salts.

The skilled person will appreciate that the aforementioned salts include ones wherein the counterion is optically active, for example d-lactate, or racemic, for example dl-tartrate.

For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of Formula (I) may be prepared by one or more of three methods:
(i) by reacting the compound of Formula (I) with the desired acid;
(ii) by removing an acid-labile protecting group from a suitable precursor of the compound of Formula (I) using the desired acid; or
(iii) by converting one salt of the compound of Formula (I) to another by reaction with an appropriate acid or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of Formula (I) or pharmaceutically acceptable salts thereof may exist in both unsolvated and solvated forms. The term ‘solvate’ is used herein to describe a molecular complex comprising a compound of Formula (I) or a pharmaceutically acceptable salt thereof and one or more pharmaceutically acceptable solvent 35 molecules, for example, ethanol. The term ‘hydrate’ is employed when said solvent is water. Pharmaceutically acceptable solvates in accordance with the invention include those wherein the solvent of crystallization may be isotopically substituted, e.g. D20, d₆-acetone and d₆-DMSO.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995), incorporated herein by reference. Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) of compounds of Formula (I) or pharmaceutically acceptable salts thereof wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004), incorporated herein by reference. For a general review of multi-component complexes, see J Pharm Sci, 64 (8), 1269-1288, by Haleblian (August 1975), incorporated herein by reference.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970), incorporated herein by reference.

The compounds of the invention may be administered as prodrugs. Thus certain derivatives of compounds of Formula (I) which may have little or no pharmacological activity themselves can, when administered into or onto the body, be converted into compounds of Formula (I) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs can, for example, be produced by replacing appropriate functionalities present in a compound of Formula (I) with certain moieties known to those skilled in the art as ‘pro-moieties’ as described, for example, in “Design of Prodrugs” by H Bundgaard (Elsevier, 1985).

Examples of prodrugs include phosphate prodrugs, such as dihydrogen or dialkyl (e.g. di-tert-butyl) phosphate prodrugs. Further examples of replacement groups in accordance with the foregoing examples and examples of other prodrug types may be found in the aforementioned references.

Also included within the scope of the invention are metabolites of compounds of Formula (I), that is, compounds formed in vivo upon administration of the drug.

Examples of metabolites in accordance with the invention include:
(i) where the compound of Formula (I) contains an alkoxy group, a hydroxy derivative thereof (—(C₁-C₆)alkoxy→—OH); and
(ii) where the compound of Formula (I) contains a phenyl moiety, a phenol derivative thereof (-Ph→-PhOH).

Formula (I) contains an asymmetric cyclopropaindazolyl moiety and is stereospecifically defined (as the ‘4aS,5aR’ stereoisomer).

The skilled person will appreciate that one or more substituents in Formula (I) may introduce one or more additional asymmetric centres. For example, an additional asymmetric centre is present in compounds of Formula (I) when each R³ therein is different. Such an asymmetric centre is found in the compound of Example 10, shown below, where the additional asymmetric carbon atom is marked by an asterisk (*):

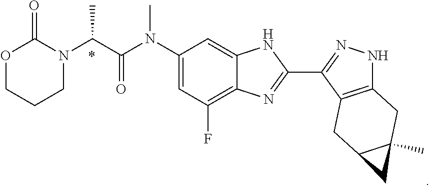 [SMILES: C[C@H](C(=O)N(C)c1cc(F)c2nc(-c3n[nH]c4c3C[C@@H]3C[C@]3(C)C4)[nH]c2c1)N1CCCOC1=O]

Compounds of the invention containing said one or more additional asymmetric centres can exist as two or more stereoisomers; included within the scope of the invention are all such stereoisomers (including epimers) of the compounds of the invention and mixtures of two or more thereof.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of Formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

Chiral chromatography using sub- and supercritical fluids may be employed. Methods for chiral chromatography useful in some embodiments of the present invention are known; see, for example, Smith, Roger M., Loughborough University, Loughborough, UK; Chromatographic Science Series (1998), 75 (Supercritical Fluid Chromatography with Packed Columns), pp. 223-249 and references cited therein.

Mixtures of stereoisomers may be separated by conventional techniques known to those skilled in the art; see, for example, “Stereochemistry of Organic Compounds” by E. L. Eliel and S. H. Wilen (Wiley, New York, 1994.

Where structural isomers are interconvertible via a low energy barrier, tautomeric isomerism (‘tautomerism’) and conformational isomerism can occur.

Tautomerism can take the form of proton tautomerism in compounds of Formula (I), as illustrated below in Formula (I) generally, and Example 1 specifically, with respect to the benzimidazole group:

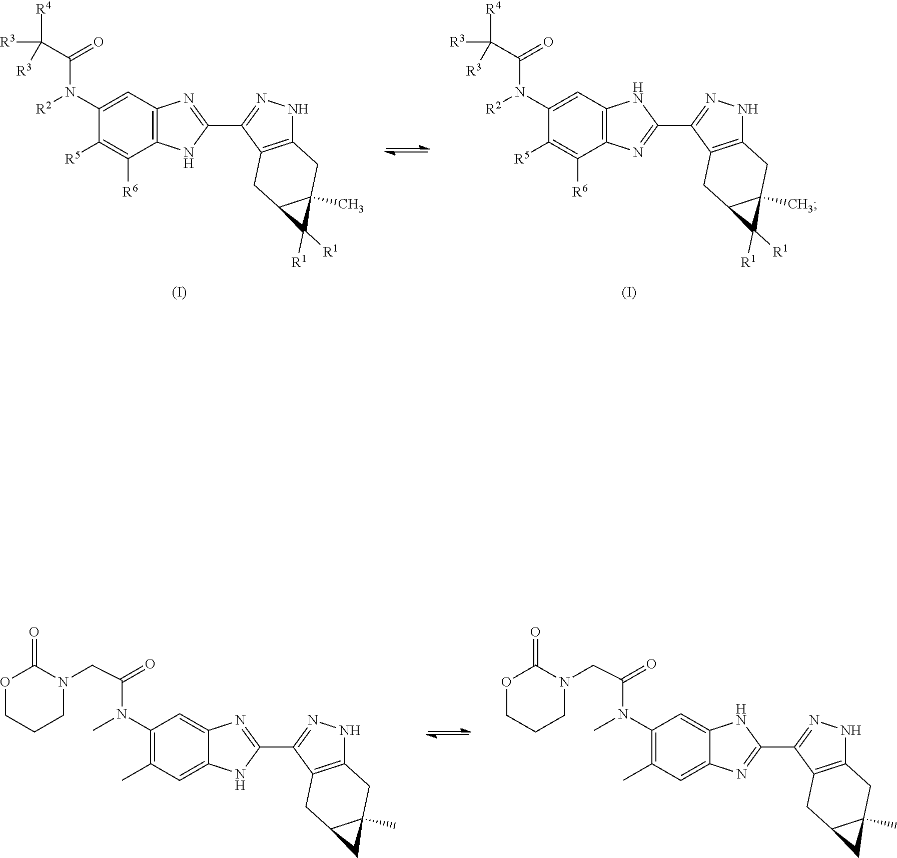

The skilled person will appreciate that proton tautomerism can also take place on the pyrazole ring in compounds of Formula (I).

While, for conciseness, the compounds of Formula (I) have been drawn herein in a single tautomeric form, all possible tautomeric forms, and mixtures thereof, are included within the scope of the invention.

Conformational isomerism is a form of stereoisomerism in which the isomers of a compound can be interconverted exclusively by rotations about single bonds. Such isomers are generally referred to as conformational isomers or conformers and, specifically, as rotamers. A “rotameric mixture”, or “mixture of rotamers”, describes a compound existing as a mixture of more than one of the possible conformational isomers. While, for conciseness, the compounds of Formula (I) have been drawn in a single conformational form, all possible conformers, and mixtures thereof, are included within the scope of the invention.

The scope of the invention includes all crystal forms of the compounds of the invention, including racemates and racemic mixtures (conglomerates) thereof. Stereoisomeric conglomerates may also be separated by the conventional techniques described herein just above.

The scope of the invention includes all pharmaceutically acceptable isotopically-labelled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention include isotopes of: hydrogen, such as ²H and ³H; carbon, such as ¹¹C, ¹³C and ¹⁴C; fluorine, such as ¹⁸F; chlorine, such as ³⁶Cl; iodine, such as ¹²³I and ¹²⁵I; nitrogen, such as ¹³N and ¹⁵N; oxygen, such as ¹⁵O, ¹⁷O and ¹⁸O.

Certain isotopically-labelled compounds of the invention, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium (D), i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁵O and ¹³N, can be useful in Positron Emission Topography (PET) studies for examining substrate receptor occupancy.

Isotopically-labeled compounds of Formula (I) can generally be prepared by conventional techniques known to those skilled in the art or by processes analogous to those described in the accompanying examples and preparations using an appropriate isotopically-labeled reagent in place of the non-labeled reagent previously employed.

Also within the scope of the invention are intermediate compounds as hereinafter defined, all salts, solvates and complexes thereof, and all solvates and complexes of salts thereof as defined hereinbefore for compounds of Formula (I). The invention includes all polymorphs of the aforementioned species and crystal habits thereof.

When preparing a compound of Formula (I) in accordance with the invention, a person skilled in the art may routinely select the form of intermediate which provides the best combination of features for this purpose. Such features include the melting point, solubility, processability and yield of the intermediate form and the resulting ease with which the product may be purified on isolation.

The compounds of the invention may be prepared by any method known in the art for the preparation of compounds of analogous structure. In particular, the compounds of the invention can be prepared by the procedures described by reference to the schemes that follow, or by the specific methods described in the examples, or by similar processes to either.

The skilled person will appreciate that the experimental conditions set forth in the schemes that follow are illustrative of suitable conditions for effecting the transformations shown, and that it may be necessary or desirable to vary the precise conditions employed for the preparation of compounds of Formula (I). It will be further appreciated that it may be necessary or desirable to carry out the transformations in a different order from that described in the schemes, or to modify one or more of the transformations, to provide the desired compound of the invention.

Compounds of the present invention contain two or more stereogenic centers, with the stereochemical designation (R) or (S). The skilled person will appreciate that all the synthetic transformations can be conducted on either enantioenriched or racemic compounds, and that the resolution to the desired stereoisomer may take place at any point in the synthesis, using well known methods described herein and/or known in the art.

In addition, the skilled person will appreciate that it may be necessary or desirable at any stage in the synthesis of compounds of the invention to protect one or more sensitive groups, so as to prevent undesirable side reactions. In particular, it may be necessary or desirable to protect hydroxyl, carboxyl and/or amino groups. The protecting groups used in the preparation of the compounds of the invention may be used in conventional manner; see, for example, those described in ‘Greene's Protective Groups in Organic Synthesis’ by Theodora W Greene and Peter G M Wuts, fifth edition, (John Wiley and Sons, 2014), incorporated herein by reference, and in particular chapters 2, 5 and 7 respectively, which also describes methods for the removal of such groups.

In the following general processes and unless otherwise stated:
R¹ to R⁶ are as previously defined for a compound of Formula (I);
R is alkyl, such as ethyl, or in the case of Formulae 3 and 4, two R may be taken together with the oxygen atoms to which they are attached to form a cyclic acetal;
PG is a suitable amino protecting group, such as a silyl ether (e.g. SEM), an alkoxy carbonyl (e.g. BOC), acetyl (Ac), benzyl (e.g. PMB) or dihydropyran (DHP) protecting group; and
X is F or Cl.

A substituted pyrazole of Formula 11 may be prepared as shown in Scheme 1.

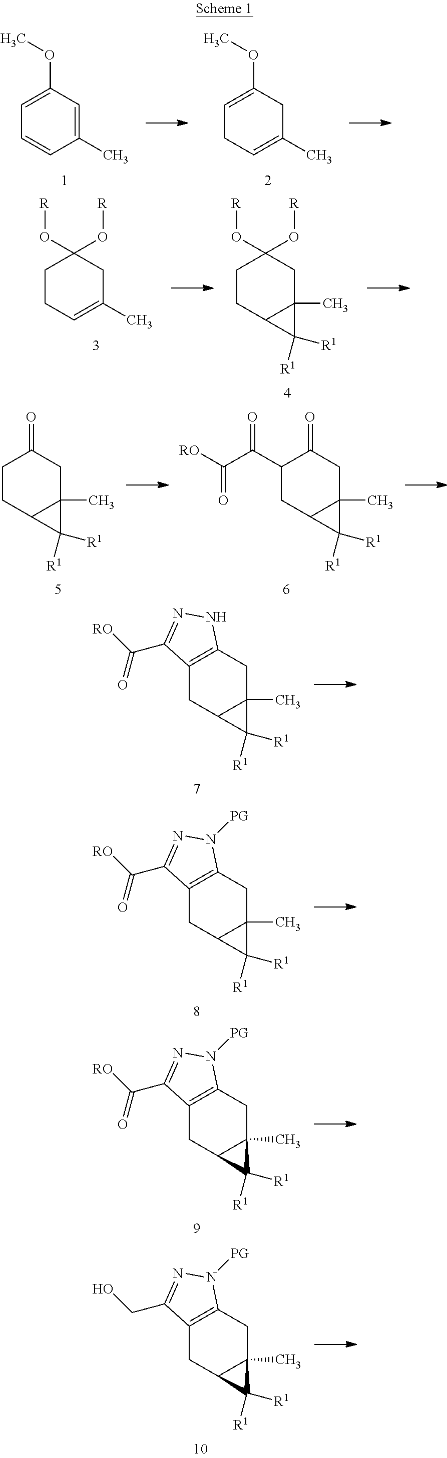

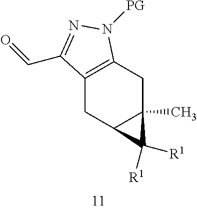

Compound 1 (3-methoxytoluene) can be reduced to the corresponding 1,4-diene Compound 2 by a Birch reduction (Mander, L. N. Comprehensive Organic Synthesis; Trost, B. M. and Fleming, I., Ed.; Pergamon: Oxford, 1991, Vol. 8, pp. 489-521), using an alkali metal such as Li or Na in liquid ammonia at temperatures below −30° C.

Preparation of an olefinic acetal of Formula 3 from the 1,4-diene Compound 2 can proceed under catalytic acid conditions, e.g. using pTSA or CSA in the presence of alkyl primary alcohols such as MeOH or EtOH, or a diol such as ethylene glycol, with or without a solvent such as DCM or other aprotic solvent, at a temperature between 0-100° C., such as 0-25° C.

Conversion of an olefin of Formula 3 into a cyclopropane of Formula 4 may proceed via dihalocarbene addition or Simmons-Smith cyclopropanation (Charette, A. B.; Beauchemin, A. Simmons-Smith Cyclopropanation Reaction. Org. React. 2001, 58, p 1-415).

Deprotection of an acetal of Formula 4 to give a ketone of Formula 5 may be performed under acidic conditions, e.g. using HCl, H₂SO₄ or an organic acid such as pTSA, in a mixture of water and solvent such as THF.

Preparation of a diketone of Formula 6 can be achieved by reacting a ketone of Formula 5 with: i) a dialkyl oxalate and 1-3 equivalents of a strong base, such as LDA, LiHMDS or KOtBu, in a polar aprotic solvent such as THF, at −78° C. to 25° C.; or ii) with an alkoxide in a corresponding alcoholic solvent (e.g. EtONa in ethanol) at temperatures between 0° C. and reflux.

Condensation of a diketone of Formula 6 with hydrazine or hydrazine hydrate, in a protic solvent such as MeOH or EtOH, at 25° C. to reflux, can provide a pyrazole of Formula 7. A hydrazine salt, such as the HCl salt, may also be used together with a corresponding molar equivalent of inorganic (e.g. K₂CO₃) or organic (e.g. Et3N or iPr₂NEt) base.

Protection of a pyrazole of Formula 7 can be performed with SEM-Cl, DHP or another suitable protecting group to deliver a pyrazole of Formula 8, resolution of which to deliver the corresponding enantiomer of Formula 9 can be performed by supercritical fluid chromatography with the use of a chiral solid phase.

Reduction of an ester of Formula 9 to an alcohol of Formula 10 may be performed using LAH, in an aprotic solvent such as THF, at temperatures between 0° C. and reflux.

Oxidation of an alcohol of Formula 10 to an aldehyde of Formula 11 can be effected by: i) using an agent, such as PCC, PDC, or MnO₂, in an aprotic solvent; or ii) by catalysis, for example by using TEMPO/bleach and TPAP/NMO (Caron, S., Dugger, R. W., Gut Ruggeri, S., Ragan, J. A., Brown Ripin, D. H., Chem. Rev. 2006, 106, 2943-2989) or Swern oxidation conditions.

A compound of Formula (I) may be prepared as shown in Scheme 2, wherein R is H or PG.

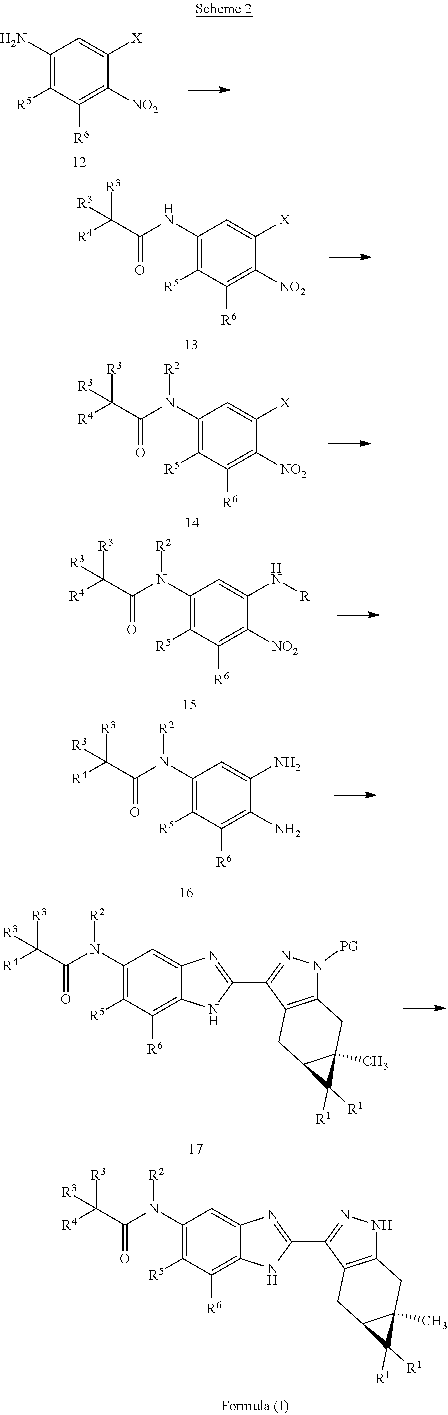

A 4-nitroaniline of Formula 12 may be acylated to provide an amide of Formula 13 with a carboxylic acid using standard amide coupling reagents such as EDCl, HATU, HBTU, or T3P; or by reaction with an alternate acylating agent, such an acid chloride, acid anhydride or acyl imidazole, in a solvent such as DCM or DMF, in the presence of an organic base such as Et₃N, at a temperature between 0° C. and reflux.

Alkylation of an amide of Formula 13 to provide an amide of Formula 14 may be effected with an alkylating agent such as an alkyl halide or tosylate, in the presence of a base such as KOtBu or LiHMDS, in a polar aprotic solvent such as DMF or THF.

A compound of Formula 14 can be converted to an amine of formula 15 (wherein R is H, benzyl or substituted benzyl) via an aromatic nucleophilic substitution (SNAr) reaction with a nitrogen nucleophile such as ammonia, benzyl amine or substituted benzyl amine; at 25 to 100° C.; in the presence of a base, such as an inorganic base (e.g. sodium-, potassium-, or cesium carbonate, bicarbonate, hydroxide, or acetate), or an organic amine base such as Et₃N; in a polar aprotic solvent such as THF, DMF, DMSO or NMP, or a protic solvent such as water, MeOH, EtOH or isopropanol, or a mixture thereof.

Reduction of a nitro aniline of Formula 15 (with concomitant deprotection, as required) can be performed under hydrogenation conditions with Pd catalyst, such as 10% Pd/C under 1-3 atm H₂, in an alcoholic solvent such as MeOH or EtOH, at a temperature between 20 and 60° C., to deliver ortho-diamines of Formula 16. Alternatively, when R=H, reduction of the nitro group can be effected by use of a metal such as Zn or Fe in AcOH as solvent or mixture of organic solvent such as THF with aqueous ammonium chloride, at a temperature between 20-100° C.

A diamine of Formula 16 can be condensed with an aldehyde of Formula 11 in a polar solvent, such as DMF with 0-5 eq DMSO, with an oxidant such as Na₂S₂O₅, at a temperature between 90 and 150° C., to deliver a benzimidazole of Formula 17. Alternatively, the condensation of compounds of Formulae 16 and 11 can proceed in the presence aqueous NaHSO₃, and EtOH or other alcoholic solvent, at 60° C. to reflux.

Removal of the protecting group in a compound of Formula 17 to deliver the corresponding compound of Formula (I) may be performed under conditions well known to the skilled person. For instance, when PG=SEM, the protecting group may be removed by use of TFA in DCM, optionally with added Et₃SiH.

The skilled person will appreciate that a compound of Formula 15 (and subsequently compounds of Formulae 16, 17 and (1)) wherein R² is H may be prepared according to Scheme 2 directly from a compound of Formula 13.

By processes directly corresponding to those described in Scheme 2, a compound of Formula (I) may also be prepared from a 3-nitro aniline of Formula 18, according to Scheme 3.

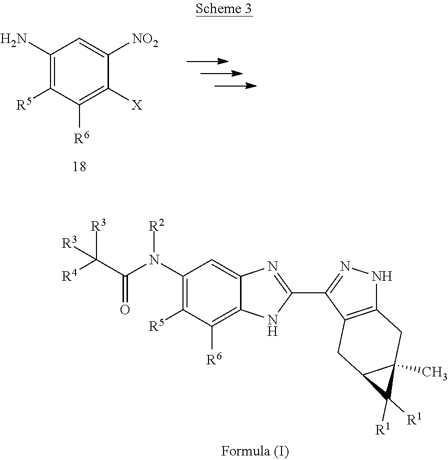

A compound of Formula (I) may also be synthesized according to Scheme 4, wherein R is H or PG.

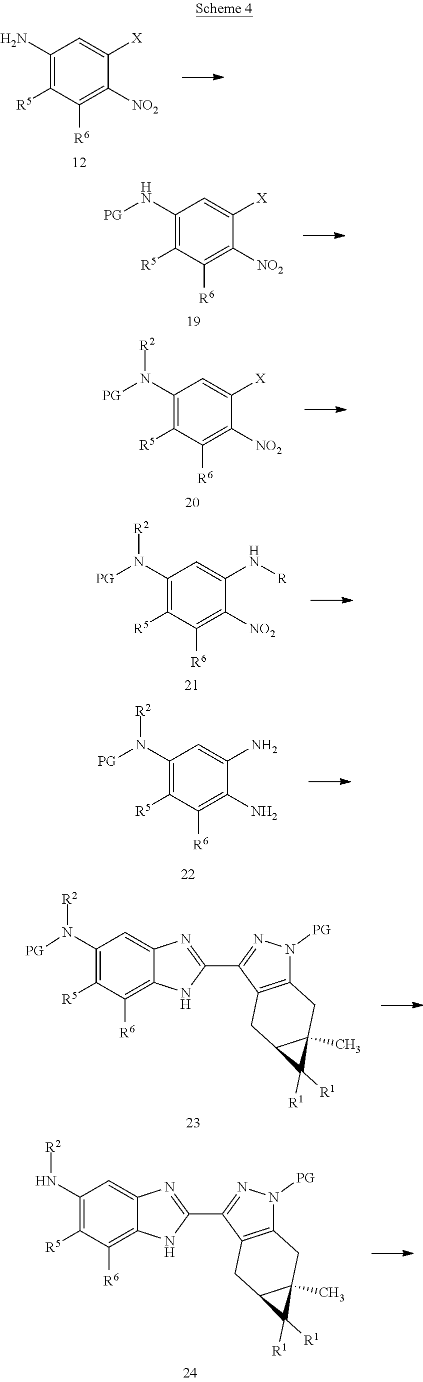

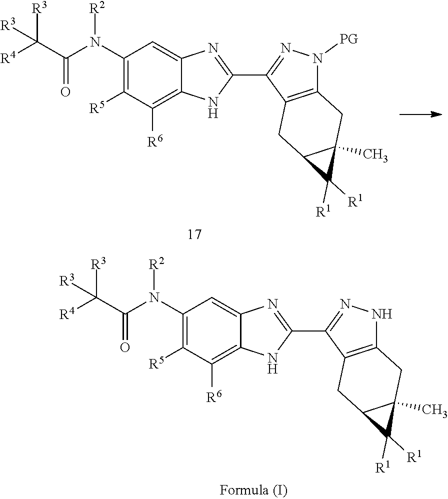

A 4-nitro aniline of Formula 12 may be N-protected with an appropriate protecting group such as BOC or Ac to deliver a compound of Formula 19, which in turn may be N-alkylated with an alkyl halide, as described in Scheme 2 above for the preparation of a compound of Formula 14, to deliver a compound of Formula 20.

A compound of Formula 20 may be substituted under the SNAr conditions described above in Scheme 2 for the preparation of an amine of Formula 15 to give a compound of Formula 21 (wherein R is H, benzyl or substituted benzyl); which in turn may be reduced, e.g. under the conditions described above in Scheme 2 for the preparation of a compound of Formula 16, to give a diamine of Formula 22; and the diamine finally condensed with an aldehyde of Formula 11 to deliver an orthogonally protected compound of Formula 23.

Selective deprotection of the aniline protecting group of a compound of Formula 23 to deliver an aniline of Formula 24 can be achieved by reaction with ZnBr₂ or TMSOTf (PG=BOC), in a non-polar solvent such as DCM; or by basic hydrolysis with aq. NaOH or KOH, in MeOH or EtOH, at reflux (PG=Ac).

An aniline of Formula 24 may be acylated under the conditions described above in Scheme 2 for the preparation of a benzimidazole of Formula 17, and the benzimidazole subsequently deprotected to provide a compound of Formula (I) under conditions well known to the skilled person, such as those described in Scheme 2 for the preparation of Formula (I).

A compound of Formula (I) may also be synthesized according to Scheme 5.

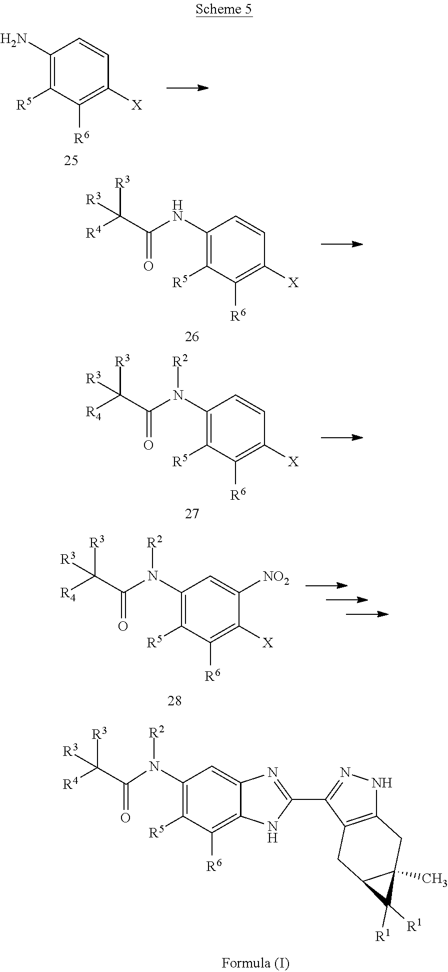

An aniline of Formula 25 may be acylated to provide an amide of Formula 26 with a carboxylic acid using standard amide coupling reagents such as EDCl, HATU, HBTU, or T3P; or by reaction with an alternate acylating agent, such an acid chloride, acid anhydride or acyl imidazole, in a solvent such as DCM or DMF, in the presence of an organic base such as Et₃N, at a temperature between 0° C. and reflux.

Alkylation of an amide of Formula 26 to provide an amide of Formula 27 may be effected with an alkylating agent such as an alkyl halide or tosylate, in the presence of a base such as KOtBu or LiHMDS, in a polar aprotic solvent such as DMF or THF.

An amide of Formula 27 may be nitrated by standard conditions including potassium nitrate in conc. sulfuric acid at a temperature between −20° C. and 50° C. to provide a nitro-arene of Formula 28.

A compound of Formula 28 may be converted into a compound of Formula (I) by processes corresponding to those described in Scheme 2 for the preparation of a compound of Formula (I) from a compound of Formula 14.

The skilled person will appreciate that a compound of Formula 28 (and subsequently a compound of Formula (I)) wherein R² is H may be prepared according to Scheme 5 directly from a compound of Formula 26.

An aniline of Formula 25 where R⁵ is (C₁-C₆)alkoxy may be synthesized according to Scheme 6.

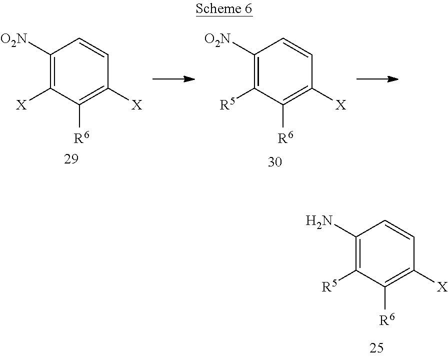

A nitro-arene of Formula 29 may be substituted under conditions of aromatic nucleophilic substitution, such as described in Scheme 4 for the preparation of a compound of formula 21, to give a compound of Formula 30 where R⁵ is alkoxy.

Reduction of a nitro arene of Formula 30 can be performed under hydrogenation conditions with Pd catalyst, such as 10% Pd/C under 1-3 atm H₂, in an alcoholic solvent such as MeOH or EtOH, at a temperature between 20 and 60° C., to deliver and aniline of Formula 25. Alternatively, reduction of the nitro group can be effected by use of a metal such as Zn or Fe in AcOH as solvent or mixture of organic solvent such as THF with aqueous ammonium chloride, at a temperature between 20-100° C.

A compound of Formula 21 may also be synthesized according to Scheme 7, wherein R is H, benzyl or substituted benzyl.

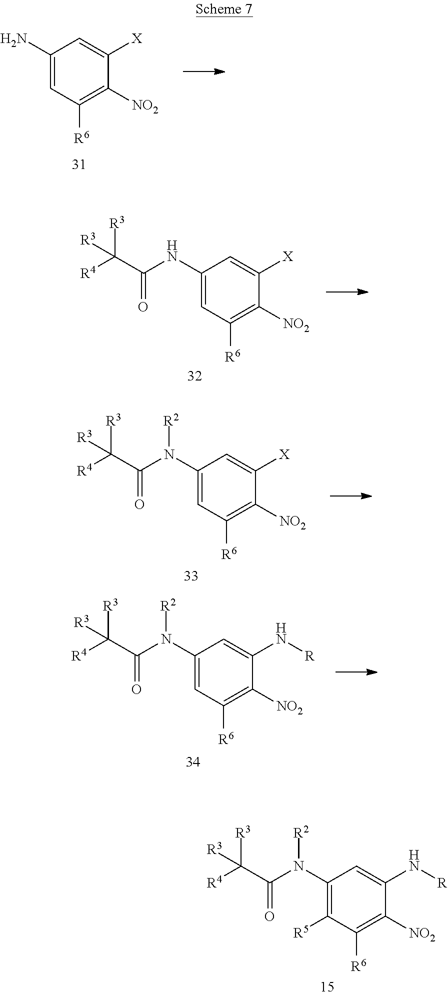

A 4-nitroaniline of Formula 31 may be acylated to provide an amide of Formula 32 with a carboxylic acid using standard amide coupling reagents such as EDCl, HATU, HBTU, or T3P; or by reaction with an alternate acylating agent, such an acid chloride, acid anhydride or acyl imidazole, in a solvent such as DCM or DMF, in the presence of an organic base such as Et₃N, at a temperature between 0° C. and reflux.

Alkylation of an amide of Formula 32 to provide an amide of Formula 33 may be effected with an alkylating agent such as an alkyl halide or tosylate, in the presence of a base such as KOtBu or LiHMDS, in a polar aprotic solvent such as DMF or THF.

A nitro aniline of Formula 34 may be prepared by substitution of X in a compound of Formula 33 with a nitrogen nucleophile, such as ammonia, benzyl amine or substituted benzyl amine, at 25 to 100° C., either neat or in a solvent such as DMF or THF.

A nitro aniline of Formula 34 may be treated with a reagent such as N-bromo succinimide in a solvent such as DMF under conditions of electrophilic aromatic substitution to provide a nitro aniline of Formula 15 where R⁵=Br.

The skilled person will appreciate that a compound of Formula 34 (and subsequently compounds of Formulae 15 and (1)) wherein R² is H may be prepared according to Scheme 7 directly from a compound of Formula 32.

A compound of Formula (I) may also be prepared from an aniline of Formula 35, according to Scheme 8, by processes directly corresponding to those described in Scheme 5.

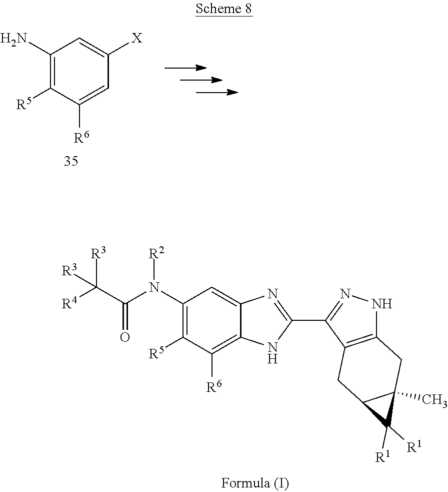

A compound of Formula 17 where R² is methyl may also be synthesized according to Scheme 9.

A diamine of Formula 36 can be condensed with an aldehyde of Formula 11 in a polar solvent, such as DMF with 0-5 eq DMSO, with an oxidant such as Na₂S₂O₅, at a temperature between 90 and 150° C., to deliver a benzimidazole of Formula 37. Alternatively, the condensation of compounds of Formulae 36 and 11 can proceed in the presence aqueous NaHSO₃, and EtOH or other alcoholic solvent, at 60° C. to reflux.

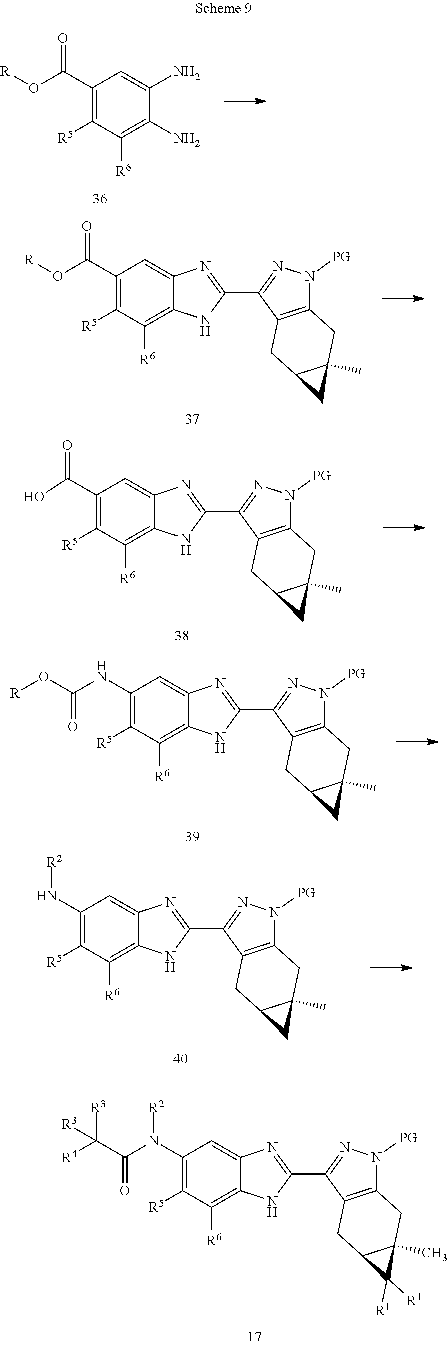

An ester of Formula 37 may be hydrolyzed using aqueous lithium, sodium or potassium hydroxide in a solvent such as MeOH, EtOH or THF, or mixture thereof, at a temperature between 20 C and reflux to provide an acid of Formula 38.

Preparation of a carbamate of Formula 39 may proceed from an acid of Formula 38 by treatment with diphenyl phosphoyl azide in a solvent such as toluene in the presence of a base such as triethylamine and an alcohol such as tert-butyl alcohol or alternative alcohols such as methanol, ethanol and benzyl alcohol at a temperature between 60° C. and 120° C.

Reduction of a carbamate of Formula 39 to an amine of Formula 40 where R² is methyl may be performed using LiAlH₄, in an aprotic solvent such as THF, at temperatures between 0° C. and reflux.

An amine of Formula 40 may be acylated to provide an amide of Formula 17 where R² is methyl with a carboxylic acid using standard amide coupling reagents such as EDCl, HATU, HBTU, or T3P; or by reaction with an alternate acylating agent, such an acid chloride, acid anhydride or acyl imidazole, in a solvent such as DCM or DMF, in the presence of an organic base such as Et₃N, at a temperature between 0° C. and reflux.

Compounds of Formulae 1, 12, 18, 25, 29, 31, 35 and 36 may be acquired from commercial sources, prepared by analogy with literature methods, or obtained by the methods described in the Experimental section that follows or variations of the same, well known to the skilled person.

All new processes for preparing compounds of Formula (I) or a pharmaceutically acceptable salts thereof, and corresponding new intermediates employed therein, form further aspects of the present invention.

Compounds of the invention intended for pharmaceutical use may be administered in amorphous or crystalline form or may exist in a continuum of solid states ranging from fully amorphous to fully crystalline. They may be obtained, for example, as solid plugs, powders, or films by methods such as precipitation, crystallization, freeze drying, spray drying, or evaporative drying. Microwave or radio frequency drying may be used for this purpose.

Compounds of the invention may be administered by any suitable route in the form of a pharmaceutical composition adapted to such a route, and in a dose effective for the treatment intended. Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the mode of administration, the effect of the excipient on solubility and stability, and the nature of the dosage form.

Modes of administration for compounds of the invention include oral, parenteral, topical, rectal, vaginal, ocular and aural administration.

Oral administration may involve swallowing, so that a compound of the invention enters the gastrointestinal tract, or buccal or sublingual administration, such that the compound enters the bloodstream directly from the mouth.

Parenteral administration may involve injecting a compound of the invention into the bloodstream, muscle or an internal organ, where the injection may be intravenous, intraarterial, intraperitoneal, intrathecal, intraventricular, intraurethral, intrasternal, intracranial, intramuscular or subcutaneous. Parenteral administration may employ needle (including microneedle) injectors, needle-free injectors and infusion techniques.

Topical administration is preferred and includes:
administration to the skin, nail, hair, claw, hoof, mucosa;
dermal or transdermal administration;
intranasal administration or administration by inhalation;
rectal or vaginal administration; and
administration directly to the eye or ear.

The term “transdermal administration” refers to the diffusion of a compound of the invention across the barrier of the skin, nail, hair, claw or hoof resulting from topical administration or other application of a composition. Transdermal delivery includes delivery through any portion of the skin, nail, hair, claw or hoof and absorption or permeation through the remaining portion.

Topical administration of a compound of the invention can result in distribution of the compound limited to the skin and surrounding tissues or, when the compound is removed from the treatment area by the bloodstream, can result in systemic exposure of the compound of the invention. Preferably, topical administration of a compound of the invention results in distribution of the compound limited to the skin and surrounding tissues. Where systemic exposure of the compound of the invention occurs, preferably the compound is rapidly metabolized so that systemic exposure of compound of the invention is minimized. Minimizing systemic exposure can reduce unwanted biological effects (i.e. side effects).

In another aspect the invention provides a pharmaceutical composition comprising a compound of the invention and a pharmaceutically acceptable excipient.

Pharmaceutical compositions suitable for the delivery of compounds of the invention and methods for their preparation will be readily apparent to those skilled in the art. Such compositions and preparative methods may be found in, for example, “Remington's Pharmaceutical Sciences”, 19th Edition (Mack Publishing Company, 1995).

Pharmaceutical compositions are typically prepared by mixing a compound of the invention and one or more excipients. Excipients include materials such as carbohydrates, waxes, water soluble and/or swellable polymers, hydrophilic or hydrophobic materials, gelatin, oils, solvents, water, buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and the like. Solvents may include water, ethanol, propylene glycol, polyethylene glycols (e.g., PEG400, PEG300), and mixtures thereof. The excipient(s) are chosen to facilitate manufacture, or use, of the pharmaceutical composition.

Pharmaceutical compositions may be prepared by conventional dissolution and mixing. For example, the compound of the invention may be dissolved in a solvent in the presence of one or more of the excipients described above. The dissolution rate of poorly water-soluble compounds may be enhanced by the use of a spray-dried dispersion, such as those described by Takeuchi, H., et al. in “Enhancement of the dissolution rate of a poorly water-soluble drug (tolbutamide) by a spray-drying solvent deposition method and disintegrants” J. Pharm. Pharmacol., 39, 769-773 (1987); and US2002/009494; incorporated herein by reference.

Solid dosage forms for oral administration of compounds of the invention include, for example, tablets, hard or soft capsules, lozenges, granules or powders, each containing at least one compound of the invention. In such solid dosage forms the compound of the invention is ordinarily combined with one or more pharmaceutically acceptable excipients. Solid dosage forms for oral administration such as tablets and capsules may be prepared with enteric coatings.

Liquid dosage forms for oral administration of compounds of the invention include, for example, pharmaceutically acceptable emulsions, solutions, suspensions, syrups, and elixirs containing inert diluents commonly used in the art (e.g. water). Such compositions also may comprise excipients, such as wetting, emulsifying, suspending, flavoring (e.g. sweetening), and/or perfuming agents.

Parenteral formulations of compounds of the invention are typically aqueous solutions which may contain excipients such as salts, carbohydrates and buffers (preferably buffering to a pH of from 3 to 9). Formulations for parenteral administration may also be sterile non-aqueous solutions, or dried (e.g. lyophilised) forms to be administered on reconstitution with a suitable vehicle such as sterile, pyrogen-free water.

Pharmaceutical compositions for topical or transdermal administration of a compound of the invention include ointments, pastes, creams, lotions, gels, suppositories, powders, solutions, sprays, drops, inhalants and patches. The compound of the invention is admixed under sterile conditions with a pharmaceutically acceptable topical carrier and any preservatives or buffers as may be required. Compounds that are volatile may require admixture with formulating agents or with packaging materials to assure proper dosage delivery. Compounds of the invention that have poor skin permeability may require one or more permeation enhancers, whereas compounds rapidly absorbed through the skin may require formulation with absorption-retarding agents or barriers.

The term “pharmaceutically acceptable topical carrier” refers to a carrier medium, suitable for topical application, that provides appropriate delivery of an effective amount of a compound of the invention, such as an inactive liquid or cream vehicle capable of suspending or dissolving the compound. The skilled person will appreciate that this term encompasses carrier materials approved for use in topical cosmetics as well.

The terms “permeation enhancer” relates to an increase in the permeability of the skin, nail, hair, claw or hoof to the compound of the invention, so as to increase the rate and extent of permeation of the compound. The enhanced permeation can be observed, for example, by measuring the rate of diffusion of the drug through animal or human skin, nail, hair, claw or hoof using a diffusion cell apparatus. A diffusion cell is described by Merritt et al. Diffusion Apparatus for Skin Penetration, J of Controlled Release, 1 (1984) pp. 161-162.

The ointments, pastes, creams, lotions, gels, suppositories, powders, solutions, sprays, drops, inhalants and patches for topical administration may contain, in addition to a compound of the invention, one or more pharmaceutically acceptable excipients, such animal or vegetable fats, oils, waxes, paraffins, starch, tragacanth, cellulose derivatives, polyethylene glycols, silicones, bentonites, silicic acid, talc, zinc oxide, preservatives, antioxidants, fragrances, emulsifiers, dyes, inert fillers, anti-irritants, tackifiers, fragrances, opacifiers, antioxidants, gelling agents, stabilizers, surfactants, emollients, coloring agents, preservatives, buffering agents, permeation enhancers. Such excipients should not interfere with the effectiveness of the biological activity of the active agent and not be deleterious to the epithelial cells or their function.

Transdermal administration may be achieved by means of a transdermal patch. The transdermal patch may be of the ‘reservoir and porous membrane’ type or employ a ‘matrix system’.

The solubility of compounds of compounds of the invention used in the preparation of pharmaceutical compositions may be increased by the use of appropriate formulation techniques, such as the incorporation of solubility-enhancing agents.

Pharmaceutical compositions may be formulated to be immediate and/or modified release. Conveniently compounds of the invention are formulated for immediate release

Modified release formulations include delayed-, sustained-, pulsed-, controlled-, targeted- and programmed-release. Thus compounds of the invention may be formulated as a solid, semi-solid, or thixotropic liquid for administration as an implanted depot providing modified release of the active compound. Examples of such formulations include poly(dl-lactic-coglycolic)acid (PGLA) microspheres.

The compounds of the invention may be combined with soluble macromolecular entities, such as cyclodextrin and suitable derivatives thereof, or polyethylene glycol-containing polymers, in order to improve their solubility, dissolution rate, taste-masking, bioavailability and/or stability for use in any of the aforementioned modes of administration.

For administration to human patients, the total daily dose of the compounds of the invention is typically in the range 1 mg to 10 g, such as 60 mg to 6 g, for example 100 mg to 1.5 g, depending on the mode of administration and efficacy. For example, administration may require a total daily dose of from 200 mg to 1 g, such as from 250 mg to 750 mg. The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

As noted above, the compounds of the invention are useful because they exhibit pharmacological activity in animals, i.e. inhibition of ITK. More particularly, the compounds of the invention are of use in the treatment of disorders for which an ITK inhibitor is indicated.

Preferably the animal is a mammal, more preferably a human.

Preferably the compound of the invention also inhibits TRKA.

In a further aspect of the invention there is provided a compound of the invention for use as a medicament.

In a further aspect of the invention there is provided a compound of the invention for use in the treatment of a disorder for which an ITK inhibitor is indicated.

In a further aspect of the invention there is provided use of a compound of the invention for the preparation of a medicament for the treatment of a disorder for which an ITK inhibitor is indicated.

In a further aspect of the invention there is provided a method of treating a disorder in an animal (preferably a mammal, more preferably a human) for which an ITK inhibitor is indicated, comprising administering to said animal a therapeutically effective amount of a compound of the invention.

Disorders or conditions for which an ITK inhibitor is indicated include inflammatory, autoimmune, dermatologic, eye, respiratory, joint, cardiovascular and neuroinflammatory diseases. The skilled person will appreciate that a given disease, disorder or condition may fall into more than one of the above categories.

More particularly, disorders or conditions for which an ITK inhibitor is indicated include:
inflammatory disorders, such as allergic conjunctivitis, celiac diseases, proctitis, eosinophilic gastroenteritis, mastocytosis, inflammatory bowel disease (e.g. Crohn's disease, ulcerative colitis, microscopic colitis (such as collagenous colitis or lymphocytic colitis), diversion colitis, Behcet's disease, and indeterminate colitis), nephritis, retinitis, retinopathy, myositis, vasculitis, Sjogren's syndrome, Wegener's granulomatosis, arteritis, sclerosing cholangitis, and eosinophilic esophagitis;
autoimmune disorders, such as lupus nephritis, autoimmune hepatitis, myasthenia gravis, Guillain-Barre syndrome, and Graves' disease;
eye disorders or conditions, including autoimmune diseases of the eye, keratoconjunctivitis, vernal conjunctivitis, non-infectious uveitis (e.g. uveitis associated with Behcet's disease and lens-induced uveitis), keratitis (e.g. herpetic keratitis and conical keratitis), corneal epithelial dystrophy, keratoleukoma, ocular premphigus, Mooren's ulcer, scleritis, retinitis, retinopathy, Grave's ophthalmopathy, Vogt-Koyanagi-Harada syndrome, keratoconjunctivitis sicca (dry eye), phlyctenule, iridocyclitis, sarcoidosis, endocrine ophthalmopathy, sympathetic ophthalmitis, allergic conjunctivitis, and ocular neovascularization;
dermatological conditions, such as eczema (e.g. chronic and dyshidrotic eczema), chronic itch, dermatitis (e.g. atopic, irritant contact, allergic contact, occupational, perioral, stasis, nummular, seborrheic, xerotic, eyelid, diaper, and hand dermatitis), vitiligo, alopecia areata, pruritis (e.g. chronic idiopathic pruritus), psoriasis (e.g. plaque, guttate, inverse, pustular, nail, flexural palmoplantar, facial or erythrodermic psoriasis), scleroderma, pemphigus, dermatomyositis, neurodermatitis, skin flushing, urticaria, cutaneous lupus erythematosus (e.g. acute cutaneous lupus (acute skin lupus), subacute cutaneous lupus (subacute lupus), and chronic cutaneous lupus (discoid lupus)), keloid, sunburn, hypertrophic scar, idiopathic thrombocytopenic thrombotic purpura (also known as immune thrombocytopenia purpura (ITP)), ichthyosis (e.g. ichthyosis vulgaris), epidermal hyperplasia, acne, lichen planus, lichen sclerosis, rosacea, epidermolysis bullosa, intertrigo, keratosis pilaris, urticaria (e.g. chronic spontaneous urticaria, chronic idiopathic urticaria, chronic physical urticaria), molluscum contagiosum, Netherton syndrome, Vogt-Koyanagi-Harada syndrome, Sweet's syndrome, pityriasis alba, vulvovaginitis, Sutton's nevus/nevi, post inflammatory hypopigmentation, senile leukoderma, chemical/drug-induced leukoderma, palmoplantar pustulosis, pemphigoid, and hidradenitis suppurativa;
respiratory conditions, such as rhinitis (e.g. allergic and perennial rhinitis), rhinorrhoea, nasal congestion, nasal inflammation, asthma (e.g. chronic asthma, inveterate asthma, late asthma, bronchial asthma, allergic asthma, intrinsic asthma, extrinsic asthma, and dust asthma), chronic obstructive pulmonary disease (COPD), chronic and acute bronchoconstriction, chronic bronchitis, emphysema, chronic eosinophilic pneumonia, acute lung injury (ACI), adult respiratory distress syndrome (ARDS), pulmonary vascular disease (PVD), pulmonary arterial hypertension (PAH), bronchiectasis, sinusitis, pulmonary sarcoidosis, and silicosis;
joint disorders, such as arthritis (e.g. osteoarthritis, as well as psoriatic, rheumatoid, juvenile, and gouty arthritis), spondyloarthropathy (e.g. reactive arthritis (also known as Reiter's Syndrome) and axial spondyloarthritis (including ankylosing spondylitis)), cartilage inflammation, bone degradation, and Still's disease;
cardiovascular and metabolic disorders, such as diabetes (type 1 and type 2), diabetic neuropathy, cachexia, and Celiac Sprue; and
neuroinflammatory disorders, such as lupus (e.g. CNS, systemic and discoid lupus), diabetic neuropathy, and multiple sclerosis.

Allergic contact dermatitis (ACD) is a contact dermatitis characterised by an allergic response to contact with a substance. An example of ACD is urushiol-induced contact dermatitis (also called toxicodendron dermatitis or rhus dermatitis), which is caused by the oil urushiol found in various plants, including poison ivy, poison oak, poison sumac and the Chinese lacquer tree. Other allergens that can induce ACD include chromium, gold and nickel.

Irritant contact dermatitis (ICD) is a form of contact dermatitis that can be divided into forms caused by chemical irritants and those caused by physical irritants. Common chemical irritants include acids, alkalis, latex, oils, perfumes and preservatives in cosmetics, solvents, and surfactants.

Occupational dermatitis is an ACD or ICD arising from exposure to an allergen or irritant in a work environment.

Additionally, an ITK inhibitor may be of use in treating certain viral and bacterial infections, transplant rejection, septic shock, acute or chronic graft-versus-host disease, polymyalgia rheumatica, sarcoidosis, Addison's disease and Raynaud's syndrome.

In one embodiment the disorder or condition for which an ITK inhibitor is indicated is a dermatological condition. In another embodiment the dermatological condition for which an ITK inhibitor is indicated is dermatitis. In another embodiment the dermatitis for which an ITK inhibitor is indicated is atopic dermatitis.

A compound of the invention may usefully be combined with one or more other pharmacologically active compounds. Such combinations offer the possibility of significant advantages, including patient compliance, ease of dosing and synergistic activity.

In a further aspect of the invention there is provided a compound of the invention in combination with another pharmacologically active compound, or with two or more other pharmacologically active compounds.

In such combinations the compound of the invention and other pharmacologically active compound(s) may be administered simultaneously, such as in a single dosage form (e.g. a composition for topical administration, such as a cream or an ointment), sequentially or separately.

The one or more additional therapeutic agents may be selected from any of the agents or types of agent that follow:
an agent for treating autoimmune and/or inflammatory disorders, such as, sulfasalazine, mesalazine, azathioprine, an antibody (e.g. infliximab, adalimumab, belimumab, tanezumab, ranibizumab, bevacizumab, mepolizumab certolizumab, natalizumab, and vedolizumab), 6-mercaptopurine, hydroxychloroquine, mofetil, sodium mycophenolate, leflunomide, rituxan, solumedrol, depomedrol, a non-steroidal anti-inflammatory drug (NSAID) (e.g. aspirin, ibuprofen, celecoxib, valdecoxib, WBI-1001 and MRX-6), and a corticosteroid (e.g. betamethasone, dexamethasone, and prednisone);
an agent for treating dermatological conditions, such as an immunosuppressant (e.g. cyclosporin, tacrolimus, and pimecrolimus), an antibody (e.g. infliximab, adalimumab dupilumab, omalizumab, and efalizumab), a TNF inhibitor (e.g. etanercept), a PDE4 inhibitor (e.g. crisaborole), and a topical corticosteroid (e.g. fluocinonide, mapracorat, hydrocortisone, desonide, alclometasone, triamcinolone, and desoximetasone);
an agent for treating respiratory conditions, such as oxymetazoline, rifampin, an anti-histamine (e.g. fexofenadine, loratidine, desloratidine, levocetirizine, methapyrilene, cetirizine), a leukotriene receptor antagonist (e.g. montelukast and zafirlukast), a 5-lipoxygenase activating protein (FLAP) antagonist, a muscarinic receptor antagonist (e.g. tiotropium and ipratropium), sodium cromoglycate, sodium nedocromil, a corticosteroid (e.g. budesonide, fluticasone, mometasone, dexamethasone, prednisolone, ciclesonide, and beclomethasone), a beta-2 agonist (e.g. salmeterol, albuterol, salbutamol, fenoterol, and formoterol), and an antibody (e.g. omalizumab);
an agent for treating joint disorders, such as methotrexate, azathioprine, and an NSAID (e.g. aspirin, ibuprofen, celecoxib, valdecoxib, WBI-1001 and MRX-6);
an agent for treating cardiovascular and metabolic disorders, such as ursodeoxycholic acid, chloroquine, quinacrine, methylnorephrine, phenylephrine, methoxamine, oxymetazoline, theophylline, a PDE5 inhibitor (e.g. sildenafil, vardenafil, and tadalafil), a PDE4 inhibitor (e.g. crisaborole, ibudilast, cilomilast, roflumilast, and ampremilast), and a kinin B₁ or B₂ receptor antagonist; and
an agent for treating neuroinflammatory disorder treatments, such as cyclophosphamide.

The one or more additional therapeutic agents may also be selected from any of the agents that follow:
a JAK inhibitor, such as abrocitinib, baricitinib, brepocitinib cerdulatinib, decernotinib, delgocitinib, fedratinib, filgotinib, gandotinib, ilginatinib, itacitinib, lestaurtinib, momelotinib, oclacitinib pacritinib, peficitinib, ritlecitinib, ruxolitinib, tofacitinib, upadacitinib, ATI-502, BMS-986165, JTE052, PF-06826647, SNA-152, and SHR-0302;
an aryl hydrocarbon receptor agonist such as, tapinarof;
an IRAK4 inhibitor such as PF-06650833;
a vitamin D analog, such as calcipotriene;
a retinoic acid derivative such as, alitretinoin;
a liver X receptor (LXR) selective agonist, such as VTP-38543;
an H4 receptor antagonist, such as, ZPL-389;
an NKI receptor antagonist, such as, aprepitant and tradipitant;
a CRTH2 receptor antagonist, such as, fevipiprant and OC-459;
a chymase inhibitor, such as SUN 13834;
a GATA-3 inhibitor, such as SB-011 and GR-MD-02;
an ROR inverse agonist, such as VTP-43742, ARN6039, TAK-828 and JTE-451;
an immunomodulator, such as PF-06763809; and
an inhibitor of SYK and BTK, including but not limited to, R-348, fostamatinib, mastinib, mivavotinib, sperbrutinib, fenebrutinib, cerdulatinib, ibrutinib, entospletinib and tirabrutinib.

It is within the scope of the invention that two or more pharmaceutical compositions, at least one of which contains a compound of the invention, may conveniently be combined in the form of a kit suitable for coadministration of the compositions. Thus the kit of the invention comprises two or more separate pharmaceutical compositions, at least one of which contains a compound of the invention, and means for separately retaining said compositions, such as a container, divided bottle, or divided foil packet. An example of such a kit is the familiar blister pack used for the packaging of tablets, capsules and the like. The kit of the invention is particularly suitable for administering different dosage forms (e.g. topical, oral, parenteral, etc.), for administering the separate compositions at different dosage intervals, or for titrating the separate compositions against one another. To assist compliance, the kit typically comprises directions for administration and may be provided with a so-called memory aid.

In another aspect the invention provides a pharmaceutical product (such as in the form of a kit) comprising a compound of the invention together with one or more additional therapeutically active agents as a combined preparation for simultaneous, separate or sequential use in the treatment of a disorder for which an ITK inhibitor is indicated.

It is to be appreciated that all references herein to treatment include curative, palliative and prophylactic treatment.

In the non-limiting Examples and Preparations set out below that illustrate the invention, and in the aforementioned Schemes, the following the abbreviations, definitions and analytical procedures may be referred to:
AcOH is acetic acid;
Ac₂O is acetic anhydride;
APC is allophycocyanin;
aq. is aqueous;
atm is atmosphere;
ATP is adenosine 5′-triphosphate disodium salt trihydrate;
BINAP is (2,2′-bis(diphenylphosphino)-1,1′-binaphthyl);
Boc is tert-butoxycarbonyl;
BOC₂O is BOC anhydride, di-tert-butyl dicarbonate;
br is broad;
BTFFH is fluorobis(tetramethylene)formamidinium hexafluorophosphate;
BTK is Bruton's tyrosine kinase;
° C. is degrees celcius;
CD₃OD is deutero-methanol;
CDCl₃ is deutero-chloroform;
conc. is concentrated;
CSA is camphor sulphonic acid;
δ is chemical shift;
d is doublet;
dd is doublet of doublets;
ddd is doublet of doublet of doublets;
ddq is doublet of doublet of quartets;
dt is doublet of triplets;
DAST is diethylaminosulfur trifluoride;
DCM is dichloromethane;
DCE is 1,2-dichloroethane;
Dess-Martin periodinane is 3-oxo-1,3-dihydro-1λ⁵,2-benziodoxole-1,1,1-triyl triacetate;
DHP is dihydropyran;
DMAP is 4-dimethylaminopyridine;
DMF is N,N-dimethylformamide;
DMSO is dimethyl sulfoxide;
DPPA is diphenylphosphoryl azide;
EDCl is 1-ethyl-3-(3-dimethylaminopropyl)carbodiimide hydrochloride;
ee is enantiomeric excess;
eq is equivalent;
EDTA is ethylenediaminetetraacetic acid;
ESI-MS is electrospray ionization mass spectrometry;
EtOAc is ethyl acetate;
EtOH is ethanol;
EtONa is sodium ethoxide;
Et₃N is triethylamine;
Et₃SiH is triethylsilane;
g is gram;
h is hour(s);
HATU is 1-[bis(dimethylamino)methylene]-1H-1,2,3-triazolo[4,5-b]pyridinium 3-oxide,
hexafluorophosphate;
HBTU is N,N,N′,N′-tetramethyl-O-(1H-benzotriazol-1-yl)uronium hexafluorophosphate;
HEPES is (4-(2-hydroxyethyl)-1-piperazineethanesulfonic acid);
HPLC is high pressure liquid chromatography;
iPr₂NEt is N,N-diisopropylethyl amine, also known as Hunig's base;
iPrOH is isopropanol, also known as 2-propanol;
KOAc is potassium acetate;
KOtBu is potassium tert-butoxide;
L is liter;
LAH is lithium aluminium hydride;
LCMS is liquid chromatography mass spectrometry;
LDA is lithium diisopropylamide;
LiHMDS is lithium hexamethyldisilazide, also known as lithium bis(trimethylsilyl)amide;
m is multiplet;
M is molar;
MeCN is acetonitrile;
MeNH₂ is methyl amine;
MeOH is methanol;
MHz is mega Hertz;
min is minutes;
mL is milliliter;
mm is millimeter;
mmol is millimole;
mol is mole;
MS m/z is mass spectrum peak;
MTBE is methyl tert-butyl ether;
n-BuLi is n-butyl lithium;
N, in the context of concentration, is normal
NaHMDS is sodium bis(trimethylsilyl) amide;
NaOtBu is sodium tert-butoxide;
NH₄OH is 33 M aq. ammonia;
NMP is N-Methyl-2-pyrrolidone;
NMR is nuclear magnetic resonance;
PCC is pyridinium chlorochromate;
PDC is pyridinium dichromate;
Pd₂(dba)₃ is tris(dibenzylideneacetone)dipalladium(0);
Pd/C is palladium on carbon;
Pd(dppf)Cl₂ is 1,1′-bis(diphenylphosphino)ferrocene]dichloropalladium(II);
Pd(OAc)₂ is palladium(II)acetate;
Pd(OH)₂/C is palladium(II)hydroxide on carbon;
Pd(Ph₃P)₄ is tetrakis(triphenylphosphine)palladium(0);
PE is petroleum ether;
PhCH₃ is toluene;
PMB is para-methoxybenzyl;
pTSA is p-toluenesulfonic acid monohydrate;
q is quartet;
Qphos is 1,2,3,4,5-pentaphenyl-1′-(di-tert-butylphosphino)ferrocene;
RT is room temperature;
s is singlet;
sat. is saturated;
SEM-Cl is 2-(trimethylsilyl)ethoxymethyl chloride;
SFC is supercritical fluid chromatography;
SPhos is 2-Dicyclohexylphosphino-2′,6′-dimethoxybiphenyl;
t is triplet;
tt is triplet of triplets;
tert-BuDavePhos is 2-di-tert-butylphosphino-2′-(N,N-dimethylamino)biphenyl;
t-BuOH is tert-butanol;
TCEP is tris(2-carboxyethyl)phosphine;
TFA is trifluoroacetic acid;
TFAA is trifluoroacetic anhydride;
TGA is thermogravimetric analysis;
THF is tetrahydrofuran;
TMSCF₃ is trifluoromethyltrimethylsilane;
TMSOTf is trimethylsilyl trifluoromethanesulfonate;
T₃P is propylphosphonic anhydride;
Tris is tris(hydroxymethyl)aminomethane;
μm is micrometer;
v/v is volume by volume;
w/v is volume by volume;
XantPhos is 4,5-Bis(diphenylphosphino)-9,9-dimethylxanthene; and
ZnEt₂ is diethylzinc.

Unless otherwise stated all reactions are run under a nitrogen atmosphere. RT (room temperature) is generally taken to mean approximately 22° C. (±5° C.). Unless otherwise stated, the term “concentrated” refers to the process of removal of volatile compounds such as solvents by use of a rotary evaporator under reduced pressure.

¹H NMR spectra were in all cases consistent with the proposed structures. Characteristic δ for ¹H-NMR are reported relative to residual solvent signals (for CDCl₃, δH=7.27 ppm; for DMSO-d₆, δH=2.50 ppm, for CD₃OD, δH=3.30 ppm) using conventional abbreviations for designation of major peaks. The skilled person will appreciate that tautomers may be recorded within the NMR data and some exchangeable protons may not be visible. Likewise, the skilled person will appreciate that a mixture of rotamers may be recorded within the NMR data.

Mass spectra were recorded using either ESI-MS. Where relevant and unless otherwise stated the m/z data provided are for isotopes ¹⁹F, ³⁵Cl, ⁷⁹Br and/or ⁸¹Br.

The term “chromatography” refers to silica gel chromatography with mobile phase consisting of mixtures or gradients of either EtOAc/heptane or methanol/DCM or some combination thereof.

Where silica gel chromatography, preparative HPLC or SFC chromatography have been used, the skilled person will appreciate that any suitable solvent or solvent combination may be employed to purify the desired compound.

Nomenclature for the compounds of the Preparations and Examples that follow was generated using ChemDraw Professional 19.0, Perkin Elmer, in accordance with the IUPAC (International Union of Pure and Applied Chemistry).

### Preparations

### Preparation 1: 1-Methoxy-5-methylcyclohexa-1,4-diene (Compound 2)

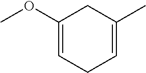

Anhydrous ammonia (1.40 kg, 82.2 mol) was bubbled into a solution of 1-methoxy-3-methylbenzene (Compound 1, 500 g, 4.09 mol) in t-BuOH (1.50 L) and THF (1.00 L) at about −55° C. Lithium sand (62.5 g, 9.00 mol) was added to the mixture while maintaining the temperature between about −50° C. and −60° C. After the addition, the reaction mixture was stirred between about −50° C. and −60° C. for about 2 h and then gradually warmed to about 15° C. The ammonia was allowed to evaporate and mixture was treated with NH₄Cl (500 g, 9.35 mol) followed by water (500 mL). The organic layer was separated and the aqueous layer was extracted with EtOAc (2×500 mL). The combined EtOAc layers were washed with brine, dried (Na₂SO₄), filtered and concentrated to provide the title Compound 2. Yield: 404 g (80%). ¹H NMR (400 MHz, CDCl₃) δ=5.41 (tt, 1H), 4.67-4.60 (m, 1H), 3.56 (s, 3H), 2.77 (ddq, 2H), 2.65-2.56 (m, 2H), 1.70 (dq, 3H).

### Preparation 2: 7-Methyl-1,4-dioxaspiro[4.5]dec-7-ene

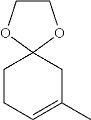

A solution of Compound 2 (500 g, 4.03 mol) in DCM (4.0 L) was treated with (±)-10-camphorsulfonic acid (46.8 g, 201 mmol) and ethylene glycol (399 g, 6.04 mol) between about −10° C. and 0° C. The mixture was stirred at about 0° C. for about 30 min. The reaction mixture was washed sequentially with sat. aq. NaHCO₃ (2 L) and then water (2×2 L), dried (Na₂SO₄), filtered and concentrated to provide the title compound. Yield: 621 g (100%). ¹H NMR (400 MHz, CDCl₃) δ=5.38 (td, 1H), 3.95 (t, 4H), 2.21-2.12 (m, 6H), 1.67 (d, 3H).

### Preparation 3: 1-Methylspiro[bicyclo[4.1.0]heptane-3,2′-[1,3]dioxolane]

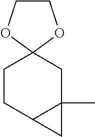

A solution of ZnEt₂ (1.00 M, 3.89 L) in DCM (2.0 L) was cooled to about 0° C. and treated with TFA (444 g, 3.89 mol, 288 mL) dropwise at about 0° C. The mixture was stirred at about 0° C. for about 30 min after which CH₂I₂ (314 mL, 3.89 mol) was added dropwise at about 0° C. The mixture was stirred at about 0° C. for about 30 min. Preparation 2 (300 g, 1.95 mol) was added dropwise, maintaining the temperature at about 0° C. and the resulting mixture was stirred at about 0° C. for about 30 min. The reaction mixture was poured into water (1 L) and extracted with DCM (3×1.75 L). The combined organic phases were dried (Na₂SO₄), filtered and concentrated to provide the title compound. Yield: 300 g (92%). ¹H NMR (400 MHz, CDCl₃) δ=3.78-3.93 (m, 4H), 2.01-2.13 (m, 1H), 1.79-1.84 (m, 1H), 1.67-1.75 (m, 2H), 1.36-1.47 (m, 1H), 1.27 (m, 1H), 1.03 (s, 3H), 0.61-0.73 (m, 1H), 0.25-0.35 (m, 2H).

### Preparation 4: 1-Methylbicyclo[4.1.0]heptan-3-one

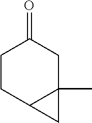

A solution of Preparation 3 (300 g, 1.78 mol) in THF (1.5 L) and water (300 mL) was treated with pTSA.H₂O (34.0 g, 178 mmol). The mixture was stirred at about 60° C. for about 3 h, cooled to RT and treated with sat. aq. NaHCO₃ solution until the pH was between 6-7. The mixture was extracted with MTBE (3×500 mL), dried (Na₂SO₄), filtered and concentrated. The crude product was purified by chromatography to provide the title compound. Yield: 85.7 g (39%). ¹H NMR (400 MHz, CDCl₃) δ=2.60-2.40 (m, 2H), 2.32-2.18 (m, 2H), 2.11-1.93 (m, 2H), 1.13 (s, 3H), 1.03-0.94 (m, 1H), 0.48-0.39 (m, 2H).

### Preparation 5

### Ethyl 2-(6-methyl-4-oxobicyclo[4.1.0]heptan-3-yl)-2-oxoacetate

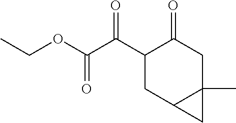 [SMILES: CCOC(=O)C(=O)C1CC2CC2(C)CC1=O]

A solution of Preparation 4 (200 g, 1.61 mol) in ethanol (1.0 L) was treated with sodium ethoxide (126 g, 1.77 mol) at about 0° C. Diethyl oxalate (259 g, 1.77 mol, 242 mL) was added at about 0° C. and the mixture was gradually warmed to RT and stirred for about 1 h. The mixture was poured into 1 N HCl (1.25 L) and extracted with DCM (3×1 L). The combined organic extracts were dried (Na₂SO₄), filtered and concentrated. The crude product was purified by chromatography to provide the title compound. Yield: 350 g (97%). LC/MS m/z (M+H)⁺=225.1

### Preparation 6

### Ethyl 5a-methyl-1,4,4a,5,5a,6-hexahydrocyclopropa[f]indazole-3-carboxylate

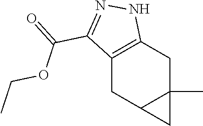 [SMILES: CCOC(=O)c1n[nH]c2c1CC1CC1(C)C2]

To a solution of Preparation 5 (350 g, 1.56 mol) in ethanol (1.5 L), was added hydrazine hydrate (79.7 g, 1.56 mol) at about 0° C. The resulting mixture was stirred at RT for about 2 h. The mixture was treated with H₂O (2.5 L) and extracted with DCM (3×2 L). The combined organic layers were dried (Na₂SO₄), filtered and concentrated. The crude product was purified by chromatography to provide the title compound 6. Yield: 200 g (58%). ¹H NMR (400 MHz, CDCl₃) δ=8.95 (s, 1H), 4.36 (q, 2H), 3.30-3.21 (m, 1H), 3.06 (d, 1H), 2.94 (dd, 1H), 2.71 (dd, 1H), 1.38 (t, 3H), 1.24 (s, 3H), 1.09-0.99 (m, 1H), 0.37-0.29 (m, 1H), 0.18 (t, 1H); LC/MS m/z (M+H)⁺=221.1.

### Preparations 6a and 6b

### Ethyl (4aR,5aS)-5a-methyl-1,4,4a,5,5a,6-hexahydrocyclopropa[f]indazole-3-carboxylate (6a) and ethyl (4aS,5aR)-5a-methyl-1,4,4a,5,5a,6-hexahydrocyclopropa[f]indazole-3-carboxylate (6b

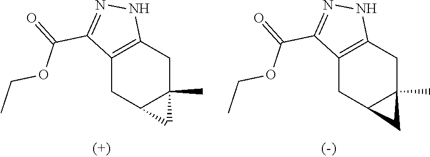

Preparation 6 was separated by chiral SFC (Chiral Tech OZ-H 250 mm×4.6 mm, 5 μm column with a mobile phase of 20% methanol (0.2% v/v 7 M NH3/methanol) and 80% CO2; flow rate 3.0 mL/min) to provide the title compounds.

Preparation 6a: retention time=3.89 min, 100% ee, [α]²⁰_{D}=+67.1 (c=4.2, methanol); LC/MS m/z (M+H)⁺=221.1.

Preparation 6b: retention time=4.76 min, 98.9% ee; [α]²⁰_{D}=−80.2 (c=4.7, methanol); ¹H NMR (400 MHz, CDCl₃) δ=4.33 (q, 2H), 3.46 (s, 2H), 3.27-3.18 (m, 1H), 3.04 (d, 1H), 2.91 (dd, 1H), 2.72-2.63 (m, 1H), 1.34 (t, 3H), 1.21 (s, 3H), 1.07-0.96 (m, 1H), 0.34-0.26 (m, 1H), 0.16 (t, 1H); LC/MS m/z (M+H)⁺=221.1.

### Preparations 7a and 7b: Ethyl (4aS,5aR)-5a-methyl-2-((2-(trimethylsilyl)ethoxy)methyl)-2,4,4a,5,5a,6-hexahydrocyclopropa[f]indazole-3-carboxylate (7a) and ethyl (4aS,5aR)-5a-methyl-1-((2-(trimethylsilyl)ethoxy)methyl)-1,4,4a,5,5a,6-hexahydrocyclopropa[f]indazole-3-carboxylate (7b

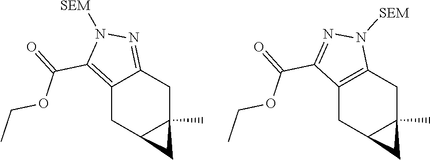

A suspension of sodium hydride (60% suspension, 19.3 g, 483 mmol) in THF (500 mL) was treated with a solution of Preparation 6b (103 g, 467.6 mmol) in THF (1.25 L) at about 0° C. After about 30 min, SEM-Cl (81.9 g, 491 mmol) was added at about 0° C. and the mixture was stirred at about 0° C. for about 3 h. The mixture was treated with sat. aq. NH₄Cl (500 mL) at about 0° C. The mixture was extracted with EtOAc (3×500 mL), washed with brine (500 mL), dried (MgSO₄), filtered and concentrated. The crude product was purified by chromatography to provide the title compounds.

Preparation 7a: Yield: 5.5 g (3.3%); ¹H NMR (400 MHz, DMSO-d₆) δ=5.63 (s, 2H), 4.27 (qt, 2H), 3.53-3.41 (m, 2H), 3.18 (d, 1H), 2.97-2.84 (m, 2H), 2.64 (d, 1H), 1.30 (t, 3H), 1.21 (s, 3H), 1.05 (dt, 1H), 0.78-0.68 (m, 2H), 0.31 (dd, 1H), 0.02 (d, 1H), −0.11 (s, 9H); LC/MS m/z (M+H)⁺=351.3

Preparation 7b: Yield: 138 g (84%); ¹H NMR (400 MHz, CDCl₃) δ=5.48-5.30 (m, 2H), 4.37 (q, 2H), 3.59-3.41 (m, 2H), 3.23 (dd, 1H), 3.07 (d, 1H), 3.02-2.86 (m, 1H), 2.73-2.58 (m, 1H), 1.36 (t, 3H), 1.23 (s, 3H), 1.13-0.95 (m, 1H), 0.85 (ddt, 2H), 0.42-0.27 (m, 1H), 0.18 (t, 1H), −0.05 (s, 9H); LC/MS m/z (M+H)⁺=351.3; [α]²⁰_{D}=−30.9 (c=1, methanol)

### Preparation 8: ((4aS,5aR)-5a-Methyl-1-((2-(trimethylsilyl)ethoxy)methyl)-1,4,4a,5,5a,6-hexahydrocyclopropa[f]indazol-3-yl)methanol

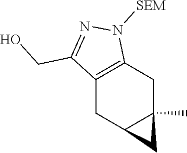 [SMILES: C[C@@]12Cc3c(c(CO)nn3COCC[Si](C)(C)C)C[C@@H]1C2]

A suspension of LiAlH₄ (14.94 g, 393.7 mmol) in THF (500 mL) at about 0° C. was treated dropwise with a solution of Preparation 7b (138 g, 393.7 mmol) in THF (1 L). The mixture was stirred at about 15° C. for about 2 h. The mixture was cooled to about 0° C. and treated sequentially by dropwise addition of H₂O (15 mL), 15% aq. NaOH (15 mL) and H₂O (30 mL), followed by MgSO₄. The resultant mixture was stirred for about 30 min, diluted with EtOAc (500 mL) and filtered. The filtrate was concentrated to provide the title compound. Yield: 110 g (91%). ¹H NMR (400 MHz, CDCl₃) δ=5.35-5.23 (m, 2H), 4.59 (dd, 2H), 3.51 (t, 2H), 3.03 (d, 1H), 2.86-2.81 (m, 2H), 2.64 (d, 1H), 1.90 (t, 1H), 1.24 (s, 3H), 1.04 (dq, 1H), 0.88 (td, 2H), 0.36 (dd, 1H), 0.23 (t, 1H), −0.03 (d, 9H); LC/MS m/z (M+H)⁺=309.3; [α]²⁰_{D}=−18.3 (c=1, methanol).

### Preparation 9: (4aS,5aR)-5a-Methyl-1-((2-(trimethylsilyl)ethoxy)methyl)-1,4,4a,5,5a,6-hexahydrocyclopropa[f]indazole-3-carbaldehyde

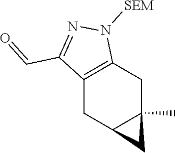 [SMILES: C[C@@]12Cc3c(c(C=O)nn3COCC[Si](C)(C)C)C[C@@H]1C2]

A solution of Preparation 8 (110.13 g, 0.36 mol) in DCM (1.5 L) was treated with activated MnO₂ (310 g, 3.57 mol) and the resulting mixture was stirred at RT for about 16 h. The mixture was filtered through a pad of Celite©. The filtrate was concentrated and the crude product was purified by chromatography to provide the title compound. Yield: 96 g (88%). ¹H NMR (400 MHz, DMSO-d₆) δ=9.86 (s, 1H), 5.52-5.38 (m, 2H), 3.50 (dd, 2H), 3.11 (dd, 2H), 2.93-2.82 (m, 1H), 2.67 (d, 1H), 1.22 (s, 3H), 1.08-1.02 (m, 1H), 0.81 (td, 2H), 0.39 (dd, 1H), 0.08 (t, 1H), −0.07 (s, 9H); LC/MS m/z (M+H)⁺=307.3; [α]²⁰_{D}=−38.9 (c=1, methanol).

### Preparation 10: 7,7-Difluoro-1-methylspiro[bicyclo[4.1.0]heptane-3,2′-[1,3]dioxolane]

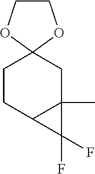

The following reaction was carried out in 26 batches in parallel. A solution of Compound 2 (150 g, 972 mmol) in THF (1.20 L) was treated with TMSCF₃ (276 g, 1.95 mol) and sodium iodide (75.8 g, 506 mmol). The mixture was stirred at about 70° C. for about 16 h. The 26 reaction mixtures were cooled to room temperature and combined. The mixture was diluted with water (10 L) and extracted with MTBE (4×3 L). The organic phase was washed with brine (8 L), dried (Na₂SO₄), filtered and concentrated to provide the title compound. Yield: 4.30 kg (83%).

### Preparation 11: 7,7-Difluoro-1-methylbicyclo[4.1.0]heptan-3-one

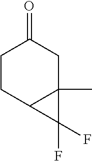

The following reaction was carried out in 5 batches in parallel. A mixture of Preparation (860 g, 4.21 mol) in THF (10 L) was treated with 3M HCl (2.6 L) at RT. The mixture was stirred at RT for about 16 hours. The 5 reaction mixtures were combined and extracted with MTBE (4×2.5 L), washed with sat. aq. NaHCO₃ (5 L) and brine (5 L). The organic phase was dried (Na₂SO₄), filtered and concentrated to provide the title compound. Yield: 3.50 kg. ¹H NMR (400 MHz, CDCl₃) δ=2.56 (br d, 1H), 2.38-2.13 (m, 4H), 2.01-1.89 (m, 1H), 1.49-1.38 (m, 1H), 1.27 (br s, 3H).

### Preparation 12: Ethyl 2-(7,7-difluoro-6-methyl-4-oxobicyclo[4.1.0]heptan-3-yl)-2-oxoacetate

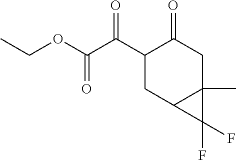 [SMILES: CCOC(=O)C(=O)C1CC2C(F)(F)C2(C)CC1=O]

The following reaction was carried out in 8 batches in parallel. A solution of Preparation 11 (250 g, 1.56 mol) in ethanol (1.25 L) was treated with sodium ethoxide (112 g, 1.65 mol) in portions at about 0° C. The resultant mixture was treated with diethyl oxalate (242 g, 1.65 mol) at about 0° C. The reaction mixture was stirred at RT for about 1 h. The 8 batches were combined. The mixture was poured into 3 M aq. HCl solution (8.00 L) and extracted with DCM (3×2 L). The organic extracts were washed with brine (5 L), dried (Na₂SO₄), filtered and concentrated to provide the title compound. Yield: 3.20 kg (98%).

### Preparation 13: Ethyl 5,5-difluoro-5a-methyl-1,4,4a,5,5a,6-hexahydrocyclopropa[f]indazole-3-carboxylate

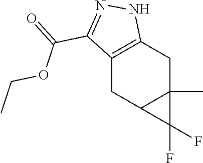 [SMILES: CCOC(=O)c1n[nH]c2c1CC1C(F)(F)C1(C)C2]

The following reaction was carried out 8 in batches in parallel. A suspension of Preparation 12 (400 g, 1.54 mol) in ethanol (2 L) was treated with hydrazine hydrate (76.9 g, 1.54 mol) at about 0° C. The reaction mixture was stirred at RT for about 16 h. The eight reaction mixtures were combined for workup. The reaction mixture was concentrated and the residue taken up in H₂O (5 L) and extracted with EtOAc (5×2 L). The combined organic extracts were dried (Na₂SO₄), filtered and concentrated. The crude product was triturated from 6:1 (v/v) EtOAc/ethanol (3 L) at RT to provide the title compound. Yield: 1.0 Kg. ¹H NMR (400 MHz, CDCl₃) δ=12.03-10.65 (br m, 1H), 4.38 (q, 2H), 3.30-3.04 (m, 3H), 2.79 (dd, 1H), 1.57 (br dd, 1H), 1.34-1.43 (m, 6H); LC/MS m/z (M+H)=257.1.

### Preparation 14: Ethyl 5,5-difluoro-5a-methyl-1-((2-(trimethylsilyl)ethoxy)methyl)-1,4,4a,5,5a,6-hexahydrocyclopropa[f]indazole-3-carboxylate

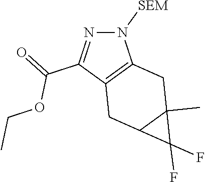 [SMILES: CCOC(=O)c1nn(COCC[Si](C)(C)C)c2c1CC1C(F)(F)C1(C)C2]

A mixture of sodium hydride (60% suspension, 1.07 g, 26.9 mmol) in THF (5 mL) cooled to about 0° C. was treated dropwise over about 15 min with a solution of Preparation 13 (5.51 g, 21.5 mmol) in THF (50 mL). The mixture was stirred at about 0° C. for about 1 h and then treated dropwise with SEM-Cl (4.76 mL, 26.9 mmol) in THF (50 mL). The resultant mixture was stirred at RT for about 48 h. The reaction mixture was treated slowly with water and extracted with EtOAc. The organic extracts were combined, washed with brine, dried (MgSO₄), filtered and concentrated to provide the title compound. Yield: 8.0 g (96%). LC-MS m/z (M+H)⁺=387.2.

### Preparation 15: (5,5-Difluoro-5a-methyl-1-((2-(trimethylsilyl)ethoxy)methyl)-1,4,4a,5,5a,6-hexahydrocyclopropa[f]indazol-3-yl)methanol

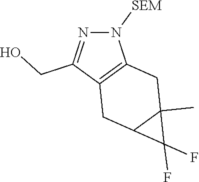 [SMILES: CC12Cc3c(c(CO)nn3COCC[Si](C)(C)C)CC1C2(F)F]

To a solution of Preparation 14 (8.0 g, 20.70 mmol) in THF (20 mL) at about 0° C. was added a solution of LiAlH₄ (29 mL, 1M in THF) dropwise. The mixture was stirred at RT for about 3 h. The mixture was cooled to about 0° C. and treated sequentially with water (1.1 mL), 15% aq NaOH (1.1 mL) and water (3.3 mL). The mixture was stirred at RT for about 15 min and treated with MgSO₄. The slurry was stirred at RT for about 15 min and filtered through a Celite© pad. The filtrate was concentrated to provide the title compound. Yield: 7.0 g (98%). LC-MS m/z (M+H)⁺=345.2.

### Preparation 16: 5,5-Difluoro-5a-methyl-1-((2-(trimethylsilyl)ethoxy)methyl)-1,4,4a,5,5a,6-hexahydrocyclopropa[f]indazole-3-carbaldehyde

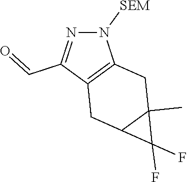 [SMILES: CC12Cc3c(c(C=O)nn3COCC[Si](C)(C)C)CC1C2(F)F]

A solution of Preparation 15 (7.0 g, 18.11 mmol) in DCM (25 mL) was cooled to about 0° C. A solution of Dess-Martin periodinane (9.6 g, 22.6 mmol) in DCM (65 mL) was added dropwise at about 0° C. The mixture was treated with water (0.33 mL) in DCM dropwise at about 0° C. The mixture was warmed to RT and stirred for about 18 h. The mixture was neutralized to about pH 7 with 1 N aq. NaOH and stirred for about 1 h. The biphasic mixture was separated. The organic phase was washed with brine, dried (MgSO₄), filtered and concentrated. The crude product was purified by chromatography to provide the title compound. Yield: 6.2 g (81%). ¹H NMR (400 MHz, CDCl₃) δ=9.98 (s, 1H), 5.53-5.35 (m, 2H), 3.61-3.43 (m, 2H), 3.27-3.05 (m, 4H), 2.82-2.66 (m, 1H), 1.42 (m, 3H), 0.98-0.80 (m, 2H), −0.08-−0.13 (m, 9H).

### Preparation 17: tert-Butyl (5-fluoro-2-methyl-4-nitrophenyl)carbamate

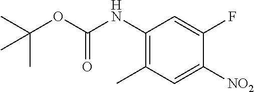 [SMILES: Cc1cc([N+](=O)[O-])c(F)cc1NC(=O)OC(C)(C)C]

The following reaction was carried out 3 in batches in parallel. A solution of 5-fluoro-2-methyl-4-nitroaniline (133 g, 781 mmol, 1 eq), DMAP (9.55 g, 78.1 mmol, 0.1 eq) and iPr₂NEt (202 g, 1.56 mol, 272 mL, 2 eq) in DCM (2 L) was treated with BOC₂O (187 g, 859 mmol) at RT. The mixture was stirred at RT for about 16 h. The 3 reaction mixtures were combined and concentrated. The residue was dissolved in EtOAc (3 L) and washed sequentially with sat. aq. NH₄Cl (1 L), sat. aq. NaHCO₃ (1 L) and brine (1 L). The organic layer was dried (Na₂SO₄), filtered and concentrated. The residue was triturated with methanol (3 L) and the solids were collected by filtration to provide the title compound. Yield: 270 g. The filtrate was concentrated and the residue was dissolved in methanol (1.5 L) and treated with K₂CO₃ (46.6 g). The mixture was stirred at RT for about 3 h. The mixture was filtered and the solids rinsed with methanol. The filtrate was concentrated and the residue purified by chromatography to provide additional title compound. Yield: 135 g. Both batches of title compound were combined. Yield: 405 g (64%). ¹H NMR (400 MHz, CDCl₃) δ=8.15 (d, 1H), 7.94-7.88 (m, 1H), 6.62 (s, 1H), 2.28 (s, 3H), 1.55 (s, 9H).

### Preparation 18: tert-Butyl (5-fluoro-2-methyl-4-nitrophenyl)carbamate

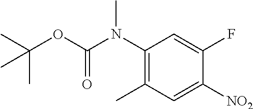 [SMILES: Cc1cc([N+](=O)[O-])c(F)cc1N(C)C(=O)OC(C)(C)C]

The following reaction was carried out 3 in batches in parallel. A solution of Preparation 17 (131 g, 486 mmol) in THF (1.9 L) was treated with KOtBu (81.9 g, 730 mmol) at about 0° C. and the mixture was stirred at about 0° C. for about 1 h. Methyl iodide (61 mL, 980 mmol) was added dropwise at about 0° C. The resulting mixture was stirred at RT for about 16 h. The 3 reaction mixtures were combined and treated with sat. aq. NH₄Cl (1.5 L) and extracted with EtOAc (2×2 L). The combined EtOAc layers were washed with brine (1.5 L), dried (Na₂SO₄), filtered and concentrated to provide the title compound. Yield: 450 g (95%). ¹H NMR (400 MHz, CDCl₃) δ=7.95 (d, 1H), 7.07 (d, 1H), 3.17 (s, 3H), 2.26 (s, 3H), 1.40 (br s, 9H).

### Preparation 19: tert-Butyl (5-amino-2-methyl-4-nitrophenyl)(methyl)carbamate

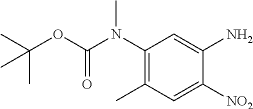 [SMILES: Cc1cc([N+](=O)[O-])c(N)cc1N(C)C(=O)OC(C)(C)C]

A solution of Preparation 18 (450 g, 1.38 mol, 1 eq) in 7 M NH₃ in methanol (7 M, 5.5 L) was heated at about 58° C. for 72 h in an autoclave. The mixture was concentrated. The residue was dissolved in EtOAc (2 L) and washed with brine (2 L). The organic layer was dried (Na₂SO₄), filtered and concentrated. The crude product was purified by chromatography to provide the title compound. Yield: 295 g (76%). ¹H NMR (400 MHz, CDCl₃) δ=7.99 (s, 1H), 6.61 (br s, 1H), 5.94 (br s, 2H), 3.14 (s, 3H), 2.14 (s, 3H), 1.55-1.23 (m, 9H).

### Preparation 20: tert-Butyl (4,5-diamino-2-methylphenyl)(methyl)carbamate

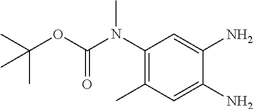 [SMILES: Cc1cc(N)c(N)cc1N(C)C(=O)OC(C)(C)C]

The following reaction was carried out 3 in batches in parallel. A solution of Preparation 19 (98 g, 349 mmol) in methanol (1 L) was treated with 10% Pd/C (10 g). The reaction mixture was stirred at about 40° C. under H₂ (3 atm) for about 24 h. The 3 reaction mixtures were combined, filtered and the solids rinsed with methanol (3×500 mL). The filtrate was concentrated to provide the title compound. Yield: 205 g (78%). ¹H NMR (400 MHz, DMSO-d₆) δ 6.32 (s, 1H), 6.27 (s, 1H), 4.37 (s, 2H), 4.29 (s, 2H), 2.96 (d, 3H), 1.89 (d, 3H), 1.44 (s, 3H), 1.28 (s, 9H). LC/MS m/z (M+H-tert butyl)⁺=195.9.

### Preparation 21: tert-Butyl methyl(5-methyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-6-yl)carbamate

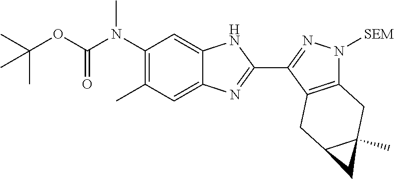 [SMILES: Cc1cc2nc(-c3nn(COCC[Si](C)(C)C)c4c3C[C@@H]3C[C@]3(C)C4)[nH]c2cc1N(C)C(=O)OC(C)(C)C]

A solution of Preparation 20 (5.53 g, 23.5 mmol) and sodium metabisulfite (2.24 g, 11.8 mmol) in DMF (124 mL) was treated with Preparation 9 (7.21 g, 23.5 mmol) and DMSO (4.6 g, 58.8 mmol) at RT. The mixture was heated at about 110° C. for about 16 h. The mixture was concentrated. The residue was treated with 3% aq. LiCl (100 mL) and extracted with EtOAc (2×100 mL). The combined EtOAc extracts were dried (MgSO₄), filtered and concentrated. The crude product was purified by chromatography to provide the title compound. Yield: 10.8 g (86%). ¹H NMR (400 MHz, CD₃OD) δ 7.46 (t, 1H), 7.38 (s, 1H), 5.53-5.42 (m, 2H), 3.63 (t, 2H), 3.40 (d, 1H), 3.26-3.11 (m, 5H), 2.77 (d, 1H), 2.33 (s, 3H), 1.56 (s, 3H), 1.36-1.30 (m, 9H), 1.18 (dd, 1H), 0.96-0.84 (m, 2H), 0.45 (dd, 1H), 0.28 (t, 1H), −0.02 (s, 9H); LC/MS m/z (M+H)⁺=538.3.

### Preparation 22: N,5-Dimethyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-6-amine

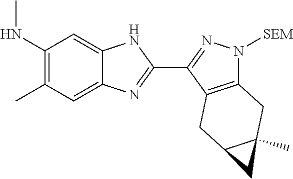 [SMILES: CNc1cc2[nH]c(-c3nn(COCC[Si](C)(C)C)c4c3C[C@@H]3C[C@]3(C)C4)nc2cc1C]

A solution of Preparation 21 (10.86 g 20.2 mmol) in DCM (135 mL) was treated with ZnBr₂ (22.7 g, 101 mmol) at about 0° C. The mixture was gradually warmed to RT and stirred for about 16 h. The mixture was poured into sat. aq. NaHCO₃ (200 mL) and extracted with DCM (2×200 mL). The combined DCM layers were dried (MgSO₄), filtered and concentrated. The crude product was purified by chromatography to provide the title compound. Yield: 7.54 g (85.3%). ¹H NMR (400 MHz, CD₃OD) δ 7.32 (s, 1H), 6.76 (s, 1H), 5.51-5.42 (m, 2H), 3.38 (t, 2H), 3.40 (d, 1H), 3.31-3.28 (m, 2H), 3.22 (s, 3H), 3.17 (m, 1H), 2.27 (s, 3H), 1.32 (s, 3H), 1.18 (m, 1H), 0.88 (m, 2H), 0.45 (dd, 1H), 0.28 (t, 1H), −0.02 (s, 9H); LC/MS m/z (M+H)⁺=438.3.

### Preparation 23: N-(5-Fluoro-2-methyl-4-nitrophenyl)acetamide

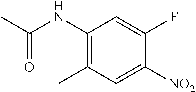

5-fluoro-2-methyl-4-nitroaniline (9.5 g, 56 mmol) was added in portions to Ac₂O (100 mL) at 15° C. The reaction mixture was stirred at about 15° C. for about 36 h. The solids were collected by filtration and rinsed with water (3×50 mL). The solids were dried to provide the title compound. Yield: 6.3 g. The filtrate was extracted with EtOAc (100 mL). The organic layer was washed with water (2×100 mL), sat. aq. Na₂HCO₃ (3×100 mL), dried (Na₂SO₄), filtered and concentrated. The crude product was purified by chromatography to provide additional title compound. Yield: 4 g. Both batches of title compound were combined to provide an overall yield of 10.3 g (89%). ¹H NMR (400 MHz, CDCl₃) δ 8.34 (d, 1H), 7.94 (d, 1H), 7.17 (s, 1H), 2.32 (s, 3H), 2.28 (s, 3H).

### Preparation 24: N-(5-Fluoro-2-methyl-4-nitrophenyl)-N-methylacetamide

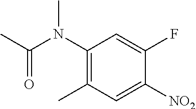 [SMILES: CC(=O)N(C)c1cc(F)c([N+](=O)[O-])cc1C]

A solution of Preparation 23 (9.3 g, 43.8 mmol) in THF (220 mL) at about 0° C. was treated with KOtBu (48.2 mL, 1M THF). The mixture was stirred at about 0° C. for about 1 h and then treated with a solution of methyl iodide (6.84 g, 48.2 mmol) in THF (20 mL). The mixture was warmed to about 15° C. and stirred for about 16 h. The mixture was treated with sat. aq. NH₄Cl (30 mL). The mixture was extracted with EtOAc (2×50 mL). The combined organic extracts were washed with brine, dried (Na₂SO₄), filtered and concentrated. The crude product was purified by chromatography to provide the title compound. Yield: 9.4 g (95%). ¹H NMR (400 MHz, CDCl₃) δ 8.04 (d, 1H), 7.13 (d, 1H), 3.18 (s, 3H), 2.30 (s, 3H), 1.82 (s, 3H); LC/MS m/z (M+H)⁺=226.9.

### Preparation 25: N-(5-Amino-2-methyl-4-nitrophenyl)-N-methylacetamide

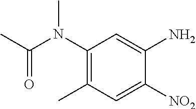 [SMILES: CC(=O)N(C)c1cc(N)c([N+](=O)[O-])cc1C]

A solution of Preparation 24 (10.3 g, 45.5 mmol) in ethanol (200 mL) at about 15° C. was treated with conc. NH₄OH (200 mL). The mixture was heated at about 50° C. and stirred for about 40 h. The ethanol was removed under reduced pressure and the suspension was filtered to collect the solids. The solids were washed with water (3×10 mL) and dried to provide the title compound. Yield: 9 g (89%). ¹H NMR (400 MHz, CDCl₃) δ 8.07 (s, 1H), 6.65 (s, 1H), 6.05 (s, 2H), 3.15 (s, 3H), 2.15 (s, 3H), 1.82 (s, 3H); LC/MS m/z (M+H)⁺=224.1.

### Preparation 26: N-(4,5-Diamino-2-methylphenyl)-N-methylacetamide

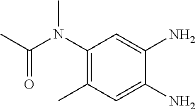

A solution of Preparation 25 (8 g, 35.8 mmol) in ethanol (10 mL) was treated with of 10% Pd/C (1.3 g). The reaction mixture was stirred at about 15° C. under H₂ (1 atm) for about 16 h. The reaction mixture was combined with that of another identical reaction conducted using 1 g Preparation 25 with similar proportions of other reagents. The combined reaction mixture was filtered and the filtrate was concentrated to provide the title compound. Yield: 7.7 g (99%). ¹H NMR (400 MHz, CDCl₃) δ 6.57 (s, 1H), 6.46 (s, 1H), 3.40 (s, 4H), 3.12 (s, 3H), 2.05 (s, 3H), 1.78 (s, 3H); LC/MS m/z (M+H)⁺=194.3.

### Preparation 27: N-Methyl-N-(6-methyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)acetamide

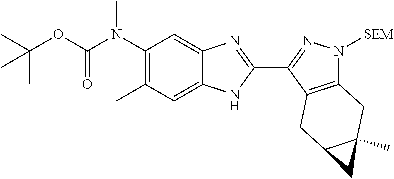 [SMILES: Cc1cc2[nH]c(-c3nn(COCC[Si](C)(C)C)c4c3C[C@@H]3C[C@]3(C)C4)nc2cc1N(C)C(=O)OC(C)(C)C]

Preparation 26 (4 g, 20.7 mmol) and sodium metabisulfite (1.97 g, 10.3 mmol) were mixed with a solution of Preparation 9 (6.84 g, 22.3 mmol) in DMF (100 mL) and DMSO (3.7 mL). The mixture was heated at about 110° C. for about 16 h. The mixture was cooled to RT and 3% aq. LiCl (150 mL) was added. The resultant solids were collected by filtration, washed with water (2×20 mL) and dried to provide the title compound. Yield: 7.9 g (80%). ¹H NMR (400 MHz, CDCl₃) δ 9.88 (s, 1H), 7.58 (s, 1H), 7.31 (s, 1H), 5.50-5.26 (m, 2H), 3.55 (t, 3H), 3.23 (s, 3H), 3.20-3.05 (m, 2H), 2.74 (d, 1H), 2.32 (s, 3H), 1.79 (s, 3H), 1.29 (s, 3H), 1.16 (dt, 1H), 0.90 (dd, 2H), 0.42 (dd, 1H), 0.26 (t, 1H), −0.03 (s, 9H); LC/MS m/z (M+H)⁺=480.4.

### Preparation 28: N-(3,5-Difluoro-4-nitrophenyl)-N-methylacetamide

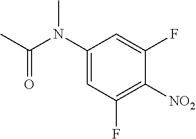 [SMILES: CC(=O)N(C)c1cc(F)c([N+](=O)[O-])c(F)c1]

A solution of 5-bromo-1,3-difluoro-2-nitrobenzene (25.0 g, 105.0 mmol) in toluene (250 mL) at RT under N₂ was treated with N-methylacetamide (11.5 g, 158 mmol), Cs₂CO₃ (68.5 g, 210 mmol), Pd₂(dba)₃ (9.62 g, 10.5 mmol), XantPhos (6.08 g, 10.5 mmol) and aluminum(III)triflate (9.96 g, 21 mmol). The mixture was heated at about 100° C. for about 15 h. The solids were removed by filtration and the filtrate was concentrated. The residue was purified by chromatography to provide the title compound. Yield: 8.75 g (36%). ¹H NMR (400 MHz, CDCl₃) δ 7.10-6.99 (m, 2H), 3.34 (s, 3H), 2.15 (s, 3H); LC/MS m/z (M+H)⁺=230.9.

### Preparation 29: N-(3-Amino-5-fluoro-4-nitrophenyl)-N-methylacetamide

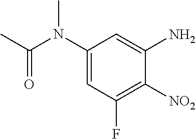 [SMILES: CC(=O)N(C)c1cc(N)c([N+](=O)[O-])c(F)c1]

A solution of Preparation 28 (8.75 g, 38.0 mmol) in ethanol (95 mL) was treated with conc. NH₄OH (24 mL). The mixture was stirred at RT for about 16 h and treated with water (120 mL). The mixture was extracted with EtOAc (2×80 mL). The combined EtOAc extracts were washed with brine, dried (Na₂SO₄), filtered and concentrated to provide the title compound. Yield: 5.70 g (66%). ¹H NMR (400 MHz, DMSO-d₆) δ 7.17 (s, 2H), 6.69 (t, 1H), 6.62 (dd, 1H), 3.15 (s, 3H), 1.99 (s, 3H). LC/MS m/z (M+H)⁺=227.9.

### Preparation 30

### N-(3,4-Diamino-5-fluorophenyl)-N-methylacetamide (30)

(00709007-2398)

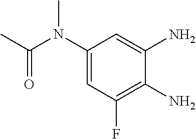

A solution Preparation 29 (5.70 g, 25.1 mmol) in ethanol (150 mL) was treated with 10% Pd/C (700 mg). The mixture was stirred under H₂ (1 atm) at RT for about 24 h. The mixture was filtered and the solids were rinsed with ethanol. The filtrate was concentrated to provide the title compound. Yield: 4.6 g (93%). ¹H NMR (400 MHz, DMSO-d₆) δ 6.32 (dd, 1H), 6.24 (dd, 1H), 5.01 (s, 2H), 4.52 (s, 2H), 3.02 (s, 3H), 1.74 (s, 3H); LC/MS m/z (M+H)⁺=198.1.

### Preparation 31: N-(7-Fluoro-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)-N-methylacetamide

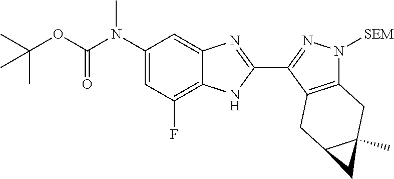 [SMILES: CN(C(=O)OC(C)(C)C)c1cc(F)c2[nH]c(-c3nn(COCC[Si](C)(C)C)c4c3C[C@@H]3C[C@]3(C)C4)nc2c1]

A solution of Preparation 9 (6.53 g, 21.3 mmol) in DMF (106 mL) at RT was treated with Preparation 30 (4.20 g, 21.3 mmol), sodium metabisulfite (2.02 g, 10.6 mmol) and DMSO (4.16 g, 53.2 mmol). The mixture was heated at about 110° C. for about 16 h and diluted with 3% aq. LiCl (50 mL). The mixture was extracted with EtOAc (2×50 mL). The EtOAc extracts were combined dried (Na₂SO₄), filtered and concentrated. The crude product was purified by chromatography to provide the title compound. Yield: 7.55 g (67%). ¹H NMR (400 MHz, CDCl₃) δ 9.99 (d, 1H), 7.45 (d, 0.5H), 7.12-7.03 (m, 0.5H), 6.87-6.71 (m, 1H), 5.40 (qd, 2H), 3.62-3.45 (m, 3H), 3.30 (s, 3H), 3.23-3.05 (m, 2H), 2.75 (d, 1H), 1.90 (s, 3H), 1.29 (s, 3H), 1.16 (s, 1H), 0.91 (ddd, 2H), 0.43 (dt, 1H), 0.27 (d, 1H), −0.02 (d, 9H); LC/MS m/z (M+H)⁺=484.4.

### Preparation 32: 7-Fluoro-N-methyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-amine

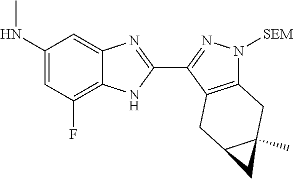 [SMILES: CNc1cc(F)c2[nH]c(-c3nn(COCC[Si](C)(C)C)c4c3C[C@@H]3C[C@]3(C)C4)nc2c1]

A solution of Preparation 31 (6.50 g, 13.44 mmol) in ethanol (51.7 mL) was treated with 5N NaOH (26.9 mL, 134 mmol). The reaction mixture was heated at about 90° C. for about 46 h. The reaction mixture was combined with that of another identical reaction conducted using 1 g Preparation 31 with similar proportions of other reagents. Water (200 mL) was added and the mixture was extracted with EtOAc (2×200 mL). The EtOAc extracts were combined, washed with brine, dried (Na₂SO₄), filtered and concentrated. The crude product was purified by chromatography to provide the title compound 32. Yield: 5.2 g (76%). ¹H NMR (400 MHz, CD₃OD) δ 6.47 (s, 1H), 6.38 (dd, 1H), 5.48-5.38 (m, 2H), 3.60 (t, 2H), 3.37-3.30 (m, 1H), 3.21-3.08 (m, 2H), 2.83-2.72 (m, 1H), 2.80 (s, 3H), 1.29 (s, 3H), 1.15 (dt, 1H), 0.88 (td, 2H), 0.42 (dd, 1H), 0.25 (t, 1H), −0.03 (s, 9H); LC/MS m/z (M+H)⁺=442.2.

### Preparation 33: N-(2-(5,5-Difluoro-5a-methyl-1-((2-(trimethylsilyl)ethoxy)methyl)-1,4,4a,5,5a,6-hexahydrocyclopropa[f]indazol-3-yl)-1-((2-(trimethylsilyl)ethoxy)methyl)-1H-benzo[d]imidazol-5-yl)-N-methylacetamide

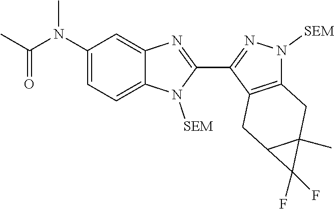 [SMILES: CC(=O)N(C)c1ccc2c(c1)nc(-c1nn(COCC[Si](C)(C)C)c3c1CC1C(F)(F)C1(C)C3)n2COCC[Si](C)(C)C]

### Step 1: Methyl 2-(5,5-difluoro-5a-methyl-1-((2-(trimethylsilyl)ethoxy)methyl)-1,4,4a,5,5a,6-hexahydrocyclopropa[f]indazol-3-yl)-1H-benzo[d]imidazole-5-carboxylate (33a

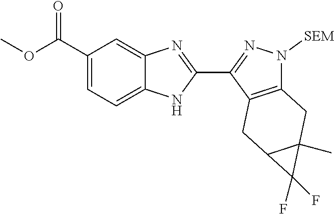

A solution of Preparation 16 (735 mg, 2.15 mmol) in DMF (10.7 mL) was treated with methyl 3,4-diaminobenzoate (411 mg, 2.47 mmol), Oxone© (429 mg, 1.40 mmol) and water (0.33 mL). The mixture was stirred at RT for about 18 h. The mixture was treated with 10% aq. sodium bisulfite and allowed to stir for several minutes and then treated with sat. aq NaHCO₃. The mixture was extracted with EtOAc. The combined EtOAc extracts were washed with brine, dried (MgSO₄), filtered and concentrated. The crude product was purified by chromatography to provide the title compound 33a. Yield: 500 mg, 48%). LC-MS m/z (M+H)⁺=489.2.

### Step 2: Methyl 2-(5,5-difluoro-5a-methyl-1-((2-(trimethylsilyl)ethoxy)methyl)-1,4,4a,5,5a,6-hexahydrocyclopropa[f]indazol-3-yl)-1-((2-(trimethylsilyl)ethoxy)methyl)-1H-benzo[d]imidazole-5-carboxylate (33b

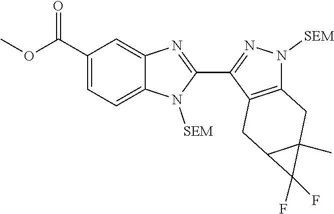

A solution of Preparation 33a (1.2 g, 2.46 mmol) in THF (20 mL) at about 0° C. was treated with sodium hydride (60% suspension, 123 mg, 3.07 mmol) and the mixture was stirred at about 0° C. for about 1 h. The reaction mixture was treated with SEM-Cl (0.54 mL, 3.07 mmol) as a solution in THF (5 mL). The reaction mixture was stirred at RT for about 18 h. The reaction mixture was treated with water and extracted with EtOAc. The combined EtOAc extracts were washed with brine, dried (MgSO₄), filtered and concentrated. The crude product was purified by chromatography to provide the title compound 33b. Yield: 600 mg (40%). LC-MS m/z (M+H)⁺=619.4.

### Step 3: 2-(5,5-Difluoro-5a-methyl-1-((2-(trimethylsilyl)ethoxy)methyl)-1,4,4a,5,5a,6-hexahydrocyclopropa[f]indazol-3-yl)-1-((2-(trimethylsilyl)ethoxy)methyl)-1H-benzo[d]imidazole-5-carboxylic acid (33c

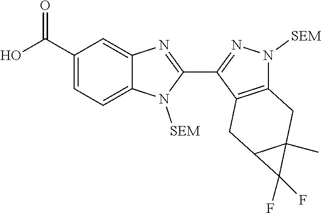 [SMILES: CC12Cc3c(c(-c4nc5cc(C(=O)O)ccc5n4COCC[Si](C)(C)C)nn3COCC[Si](C)(C)C)CC1C2(F)F]

A mixture of Preparation 33b (600 mg, 1.0 mmol) in THF (10 mL) and methanol (10 mL) was treated with 1N NaOH (6 mL). The reaction mixture was heated at about 70° C. for about 1 h. The mixture was cooled to RT and stirred for about 18 h. The volatile solvents were removed on a rotary evaporator and the resultant mixture was acidified with 1N HCl and extracted with EtOAc (3×). The combined EtOAc extracts were washed with brine, dried (MgSO₄), filtered and concentrated to provide the title compound 33c. Yield: 600 mg (100%). LC/MS m/z (M+H)⁺=605.3.

### Step 4: tert-Butyl (2-(5,5-difluoro-5a-methyl-1-((2-(trimethylsilyl)ethoxy)methyl)-1,4,4a,5,5a,6-hexahydrocyclopropa[f]indazol-3-yl)-1-((2-(trimethylsilyl)ethoxy)methyl)-1H-benzo[d]imidazol-5-yl)carbamate (33d

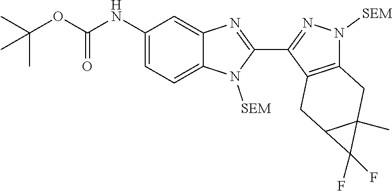 [SMILES: CC(C)(C)OC(=O)Nc1ccc2c(c1)nc(-c1nn(COCC[Si](C)(C)C)c3c1CC1C(F)(F)C1(C)C3)n2COCC[Si](C)(C)C]

A solution of Preparation 33c (1.45 g, 2.40 mmol) in toluene (30 mL) was treated with DPPA (858 mg, 3.12 mmol) and Et₃N (1.0 mL, 7.19 mmol) and t-BuOH (1.15 mmol, 12.0 mmol). The mixture was heated at reflux for about 18 h. The reaction mixture was cooled to RT and concentrated. The residue was partitioned between water and EtOAc. The EtOAc extract was dried, filtered and concentrated to provide the title compound 33d. Yield: 1.30 g (80%). LC/MS m/z (M+H)⁺=676.5.

### Step 5: 2-(5,5-Difluoro-5a-methyl-1-((2-(trimethylsilyl)ethoxy)methyl)-1,4,4a,5,5a,6-hexahydrocyclopropa[f]indazol-3-yl)-N-methyl-1-((2-(trimethylsilyl)ethoxy)methyl)-1H-benzo[d]imidazol-5-amine (33e

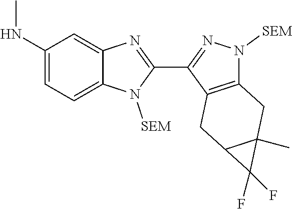 [SMILES: CNc1ccc2c(c1)nc(-c1nn(COCC[Si](C)(C)C)c3c1CC1C(F)(F)C1(C)C3)n2COCC[Si](C)(C)C]

A solution of Preparation 33d (1.79 g, 2.65 mmol) in THF (9 mL) was cooled to about 0° C. and treated with LiAlH₄ (1M in THF, 13.2 mL). The reaction mixture was then heated at reflux for about 5 h. The reaction mixture was cooled to about 0° C. and treated with 6 N aq. NaOH (6.6 mL). The mixture was then treated with EtOAc, warmed to RT, treated with MgSO₄ and filtered through Celite©. The filtrate was concentrated to provide the title compound 33e. Yield: 1.40 g (90%). LC/MS m/z (M+H)⁺=590.5.

### Step 6: N-(2-(5,5-Difluoro-5a-methyl-1-((2-(trimethylsilyl)ethoxy)methyl)-1,4,4a,5,5a,6-hexahydrocyclopropa[f]indazol-3-yl)-1-((2-(trimethylsilyl)ethoxy)methyl)-1H-benzo[d]imidazol-5-yl)-N-methylacetamide

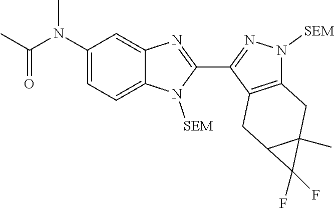 [SMILES: CC(=O)N(C)c1ccc2c(c1)nc(-c1nn(COCC[Si](C)(C)C)c3c1CC1C(F)(F)C1(C)C3)n2COCC[Si](C)(C)C]

A solution of Preparation 33e (365 mg, 0.62 mmol) in DMF (3 mL) was treated with Ac₂O (88 μL, 0.93 mmol), HATU (353 mg, 0.93 mmol) and iPr₂NEt (0.32 mL, 1.86 mmol). The reaction mixture was stirred at RT for about 18 h then diluted with water and extracted with EtOAc (2×). The combined EtOAc extracts were washed with brine, dried (MgSO₄), filtered and concentrated to provide the title compound. Yield: 390 mg (99%). LC/MS m/z (M+H)⁺=632.3.

### Preparation 34: N-(5-Methyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-6-yl)acetamide

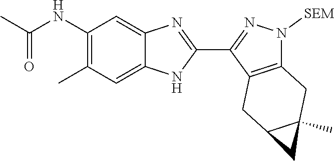 [SMILES: CC(=O)Nc1cc2nc(-c3nn(COCC[Si](C)(C)C)c4c3C[C@@H]3C[C@]3(C)C4)[nH]c2cc1C]

### Step 1: N-(4-Fluoro-2-methyl-5-nitrophenyl)acetamide (34a

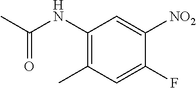

4-Fluoro-2-methyl-5-nitroaniline (16.7 g, 98.2 mmol) was added to Ac₂O (200 mL) with stirring at about 15° C., and the mixture was stirred at about 15° C. for about 16 h. The mixture was treated with water (300 mL) and extracted with EtOAc (300 mL). The organic layer was washed with sat. aq. Na₂CO₃ (2×150 mL) and brine (100 mL). The organic layer was dried (Na₂SO₄), filtered and concentrated. The residue was triturated with EtOAc/PE (v/v=1:5, 100 mL). The resulting solid was collected by filtration and dried to provide the title compound 34a. Yield: 15 g (72%). ¹H NMR (400 MHz, CDCl₃) 5=8.52 (d, 1H), 7.13 (br d, 2H), 2.35 (s, 3H), 2.25 (s, 3H)

### Step 2: N-(4-Amino-2-methyl-5-nitrophenyl)acetamide (34b

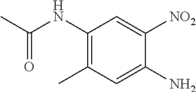

A solution of Preparation 34a (15 g, 70.7 mmol) in ethanol (300 mL) was treated with conc. NH₄OH (198 g) at about 30° C. and the mixture was stirred at about 50° C. for about 16 h. Additional conc. NH₄OH (140 g) was added and the mixture was stirred at about 50° C. for about 16 h. Additional conc. NH₄OH (46 g) was added and the mixture was stirred at about 60° C. for about 16 h. The mixture was concentrated and the solids were collected by filtration. The solids were washed with water (3×10 mL) and dried to provide the title compound 34b. Yield: 14.0 g (95%). ¹H NMR (400 MHz, CD₃OD) δ=7.97 (s, 1H), 6.81 (s, 1H), 2.19 (s, 3H), 2.13 (s, 3H)

### Step 3: N-(4,5-Diamino-2-methylphenyl)acetamide (34c

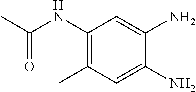

A suspension of Preparation 34b (2.50 g, 11.95 mmol) in ethanol (50 mL) was added to a suspension of 10% Pd/C (500 mg) in ethanol (10 mL). The reaction mixture was stirred at about 15° C. under H₂ (1 atm) for about 16 h. The mixture was filtered and the filtrate was concentrated to provide the title compound 34c. Yield: 2.2 g. LC/MS m/z (M+H)⁺=180.1.

### Step 4: N-(5-Methyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-6-yl)acetamide

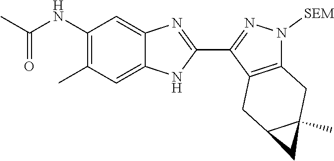 [SMILES: CC(=O)Nc1cc2nc(-c3nn(COCC[Si](C)(C)C)c4c3C[C@@H]3C[C@]3(C)C4)[nH]c2cc1C]

A solution of Preparation 34c (2.20 g, 12.28 mmol) in DMF (40 mL) was treated with sodium metabisulfite (1.17 g, 6.14 mmol), DMSO (2.18 mL, 30.7 mmol), and a solution of compound 9 (3.76 g, 12.3 mmol) in DMF (20 mL). The mixture was stirred at about 100° C. for about 16 h. The mixture was concentrated and the crude product was purified by chromatography to provide the title compound. Yield: 5.3 g (92%). LC/MS m/z (M+H)⁺=466.2.

### Preparation 35: N-Ethyl-N-(6-methyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)acetamide

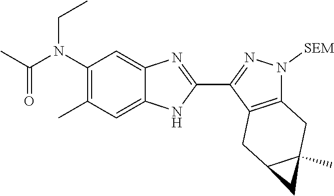 [SMILES: CCN(C(C)=O)c1cc2nc(-c3nn(COCC[Si](C)(C)C)c4c3C[C@@H]3C[C@]3(C)C4)[nH]c2cc1C]

### Step 1: N-Ethyl-5-methyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-6-amine (35a

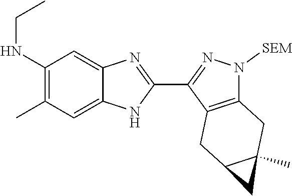

A suspension of LiAlH₄ (326 mg, 8.59 mmol) in THF (33 mL) at about 0° C. was treated with a solution of Preparation 34 (2 g, 4.3 mmol) in THF (10 mL) and stirred at RT for about 72 h. The mixture was treated with Na₂SO₄ decahydrate, followed by MgSO₄ (4 g). The mixture was stirred for about 30 min. The mixture filtered and the solids rinsed with EtOAc (2×10 mL). The filtrate was concentrated and the residue was purified by chromatography to provide the title compound 35a. Yield: 1.03 g (53%). ¹H NMR (400 MHz, CDCl₃) δ=9.55 (s, 1H), 7.51 (s, 1H), 7.10 (d, 1H), 6.57 (s, 1H), 5.43-5.27 (m, 2H), 3.60-3.50 (m, 3H), 3.28-3.13 (m, 3H), 3.09 (d, 1H), 2.72 (d, 1H), 2.25 (s, 3H), 1.35 (t, 3H), 1.28 (s, 3H), 1.14 (dt, 1H), 0.96-0.84 (m, 2H), 0.39 (dd, 1H), 0.28 (t, 1H), −0.03 (s, 9H); LC/MS m/z (M+H)⁺=452.3.

### Step 2: N-Ethyl-N-(6-methyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)acetamide

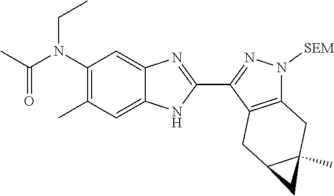 [SMILES: CCN(C(C)=O)c1cc2nc(-c3nn(COCC[Si](C)(C)C)c4c3C[C@@H]3C[C@]3(C)C4)[nH]c2cc1C]

A solution of Preparation 35a (200 mg, 0.44 mmol) and AcOH (35 mg, 0.58 mmol) in pyridine (4.4 mL) was treated with EDCl (127 mg, 0.66 mmol) at about 0° C. The reaction mixture was stirred at about 18° C. for about 13 h. The reaction mixture was treated with water (10 mL) and extracted with EtOAc (2×10 mL). The combined EtOAc extracts were dried, filtered and concentrated to provide the title compound. Yield: 220 mg (88%). LC/MS m/z (M+H)⁺=494.1.

### Preparation 36: N-(6-Methoxy-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)-N-methylacetamide

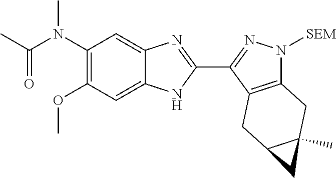 [SMILES: COc1cc2[nH]c(-c3nn(COCC[Si](C)(C)C)c4c3C[C@@H]3C[C@]3(C)C4)nc2cc1N(C)C(C)=O]

### Step 1: N-(4-Fluoro-2-methoxyphenyl)acetamide (36a

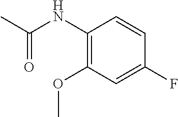

A flask charged with Ac₂O (60 mL) was treated with 4-fluoro-2-methoxyaniline (5.90 g, 39.7 mmol) added in portions at about 15° C. The reaction mixture was stirred at about 15° C. for about 24 h. Additional Ac₂O (10 mL) was added and the reaction mixture was stirred at about 15° C. for about 16 h. The reaction mixture was diluted with water (250 mL) and extracted with EtOAc (250 mL). The aqueous layer was made basic to about pH 8 with sat. aq NaHCO₃ (200 mL) and extracted with EtOAc. The combined EtOAc extracts were washed with brine, dried, filtered and concentrated. The crude product was purified by chromatography to provide the title compound 36a. Yield: 7.71 g. ¹H NMR (400 MHz, CDCl₃) δ=8.29 (dd, 1H), 7.59 (br s, 1H), 6.73-6.56 (m, 2H), 3.88 (s, 3H), 2.20 (s, 3H).

### Step 2: N-(4-Fluoro-2-methoxyphenyl)acetamide (36b

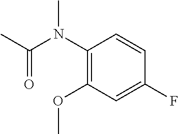

A solution of Preparation 36a (2.0 g, 10.9 mmol) in THF (156 mL) at about 0° C. was treated with sodium hydride (60% suspension, 655 mg, 16.4 mmol) and the mixture was stirred at about 15° C. for about 30 min. The reaction mixture was treated with methyl iodide (0.82 mL, 13.1 mmol). The reaction mixture was stirred about 15° C. for about 16 h. The reaction mixture was treated with sat. aq. NH₄Cl (150 mL) and extracted with EtOAc (2×200 mL). The combined EtOAc extracts were concentrated. The crude product was purified by chromatography to provide the title compound 36b. Yield: 1.99 g (92%). ¹H NMR (400 MHz, CDCl₃) δ=7.12 (dd, 1H), 6.77-6.61 (m, 2H), 3.84 (s, 3H), 3.14 (s, 3H), 1.79 (s, 3H).

### Step 3: Synthesis of N-(4-Fluoro-2-methoxy-5-nitrophenyl)-N-methylacetamide (36c

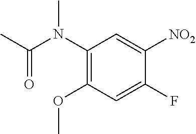 [SMILES: COc1cc(F)c([N+](=O)[O-])cc1N(C)C(C)=O]

A solution of Preparation 36b (500 mg, 2.54 mmol) in conc. H₂SO₄ (3.5 mL) at about 0° C. was treated with potassium nitrate (256 mg, 2.54 mmol) added in portions while keeping the internal reaction temperature below about 5° C. The mixture was stirred at about 0° C. for about 2 h. The reaction mixture was poured into ice water and stirred for about 10 min. The solids were collected by filtration and rinsed with water (3×10 mL) then dried to provide the title compound 36c. Yield: 500 mg (81%). ¹H NMR (400 MHz, CDCl₃) δ=8.04 (d, 1H), 6.86 (d, 1H), 3.98 (s, 3H), 3.17 (s, 3H), 1.83 (s, 3H)

### Step 4: N-(4-Amino-2-methoxy-5-nitrophenyl)-N-methylacetamide (36d

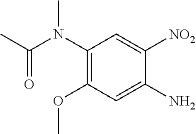 [SMILES: COc1cc(N)c([N+](=O)[O-])cc1N(C)C(C)=O]

A solution of Preparation 36c (500 mg, 2.06 mmol) in ethanol (15 mL) was treated with NH₄OH (15 mL) and the mixture was stirred at about 50° C. for about 16 h. The volatile solvents were removed on a rotary evaporator and the solids collected by filtration and rinsed with water (3×10 mL) then dried under high vacuum to provide the title compound 36d. Yield: 359 mg (73%). ¹H NMR (400 MHz, CDCl₃) δ=8.01 (s, 1H), 6.37 (br s, 2H), 6.23 (s, 1H), 3.89 (s, 3H), 3.13 (s, 3H), 1.84 (s, 3H).

### Step 5: N-(4-amino-2-methoxy-5-nitrophenyl)-N-methylacetamide (36e

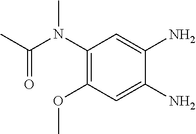

A slurry of Preparation 36d (359 mg, 1.50 mmol) and 10% Pd/C (60 mg) in methanol (10 mL) was stirred under hydrogen (1 atm) at RT for about 20 h. The mixture was filtered through Celite© and rinsed with methanol (3×). The filtrate was concentrated to provide the title compound 36e. Yield: 360 mg. LC/MS m/z (M+H)⁺=209.9.

### Step 6: N-(6-Methoxy-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)-N-methylacetamide

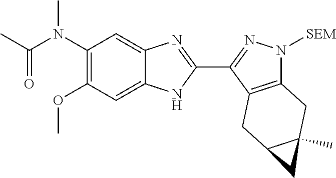 [SMILES: COc1cc2[nH]c(-c3nn(COCC[Si](C)(C)C)c4c3C[C@@H]3C[C@]3(C)C4)nc2cc1N(C)C(C)=O]

A solution of Preparation 36e (360 mg, 1.72 mmol) and sodium metabisulfite (164 mg, 0.86 mmol) in DMF (9 mL) was treated with Preparation 9 (527 mg, 1.72 mmol) and DMSO (0.31 mL, 4.30 mmol). The mixture was stirred at about 110° C. for about 16 h. The reaction mixture was poured into 3% aq. LiCl (20 mL) and extracted with EtOAc (4×40 mL). The combined EtOAc extracts were concentrated and the crude product was purified by chromatography to provide the title compound. Yield: 729 mg (86%). ¹H NMR (400 MHz, CD₃OD) δ=7.60-7.44 (m, 1H), 7.40-7.16 (m, 1H), 5.54-5.42 (m, 2H), 3.93 (s, 3H), 3.62 (t, 2H), 3.44-3.34 (m, 1H), 3.25-3.09 (m, 5H), 2.77 (br d, 1H), 1.31 (s, 3H), 1.22-1.11 (m, 1H), 0.89 (dt, 2H), 0.45 (dd, 1H), 0.27 (t, 1H), −0.03 (s, 9H).

### Preparation 37: N-(6-(2-Methoxyethoxy)-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)-N-methylacetamide

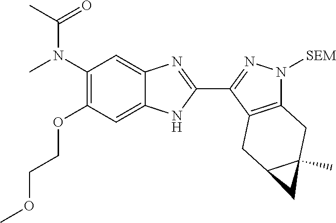

### Step 1: 4-Fluoro-2-(2-methoxyethoxy)-1-nitrobenzene (37a

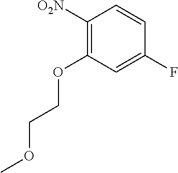

A solution of 2-methoxyethanol (2.48 mL, 31.4 mmol) in THF (40 mL) was treated with KOtBu (1 M in THF, 31.4 mL) at about 0° C. The mixture was added dropwise to a second solution of 2,4-difluoronitrobenzene in THF (40 mL) at about 0° C. The mixture was stirred at about 0° C. for about 3 h. The mixture was diluted with water (50 mL) and extracted with EtOAc (2×50 mL). The combined EtOAc extracts were dried (Na₂SO₄), filtered and concentrated to provide the title compound 37a. Yield: 6.76 g (100%). ¹H NMR (400 MHz, CDCl₃) δ=7.94 (dd, 1H), 6.83 (dd, 1H), 6.77-6.70 (m, 1H), 4.27-4.20 (m, 2H), 3.84-3.77 (m, 2H), 3.46 (s, 3H)

### Step 2: 4-Fluoro-2-(2-methoxyethoxy)aniline (37b

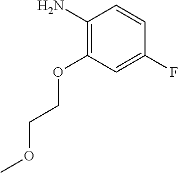

A solution of Preparation 37a (6.76 g, 31.42 mmol) in methanol (200 mL) was treated with 10% Pd/C (50% water, 600 mg) and stirred under hydrogen (1 atm) at RT for about 16 h. The mixture was filtered through Celite© and filtrate was concentrated to provide the title compound 37b. Yield: 4.53 g (78%). ¹H NMR (400 MHz, CDCl₃) δ=6.64 (dd, 1H), 6.59 (dd, 1H), 6.56-6.50 (m, 1H), 4.14-4.11 (m, 2H), 3.78-3.74 (m, 2H), 3.45 (s, 3H).

### Step 3: N-(4-Fluoro-2-(2-methoxyethoxy)phenyl)acetamide (37c

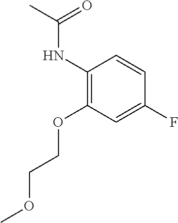 [SMILES: COCCOc1cc(F)ccc1NC(C)=O]

A mixture of Preparation 37b (4.53 g, 24.46 mmol) in Ac₂O (120 mL) was stirred at RT for about 20 h. The solids were collected by filtration and rinsed with water (50 mL). The filtrate was extracted with EtOAc (2×50 mL). The combined EtOAc extracts were washed with sat. aq. NaHCO₃ (5×50 mL) and concentrated. The crude product was purified by chromatography and combined with the previously filtered precipitate to provide the title compound 37c. Yield: 5 g (90%). ¹H NMR (400 MHz, CDCl₃) δ=8.32 (dd, 1H), 7.93 (br s, 1H), 6.79-6.62 (m, 2H), 4.21-4.11 (m, 2H), 3.79-3.69 (m, 2H), 3.47 (s, 3H), 2.19 (s, 3H).

### Step 4: N-(4-Fluoro-2-(2-methoxyethoxy)phenyl)-N-methylacetamide (37d

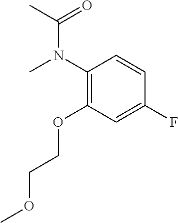 [SMILES: COCCOc1cc(F)ccc1N(C)C(C)=O]

A solution of Preparation 37c (2.0 g, 8.8 mmol) in THF (60 mL) at about 0° C. was treated with sodium hydride (60% suspension, 528 mg, 13.2 mmol) and the mixture was stirred at about 15° C. for about 30 min. The reaction mixture was treated with methyl iodide (0.61 mL, 9.9 mmol). The reaction mixture was stirred about 15° C. for about 36 h. The reaction mixture was treated with sat. aq. NH₄Cl (60 mL) and extracted with EtOAc (3×60 mL). The combined EtOAc extracts were dried (Na₂SO₄), filtered and concentrated. The crude product was purified by chromatography to provide the title compound 37d. Yield: 1.98 g (93%). ¹H NMR (400 MHz, CDCl₃) δ=7.13 (dd, 1H), 6.74-6.66 (m, 2H), 4.17-4.09 (m, 2H), 3.73 (dt, 2H), 3.42 (s, 3H), 3.16 (s, 3H), 1.81 (s, 3H).

### Step 5: N-(4-Fluoro-2-(2-methoxyethoxy)-5-nitrophenyl)-N-methylacetamide (37e

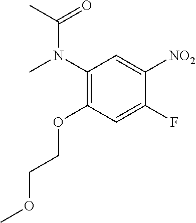 [SMILES: COCCOc1cc(F)c([N+](=O)[O-])cc1N(C)C(C)=O]

A solution of Preparation 37d (1.98 g, 8.21 mmol) in conc. H₂SO₄ (12 mL) at about 0° C. was treated with potassium nitrate (830 mg, 8.21 mmol) in portions while keeping the internal reaction temperature below about 5° C. The mixture was stirred at about 0° C. for about 2 h. The reaction mixture was poured into ice water (60 mL) and stirred for about 10 min then extracted with EtOAc (2×60 mL). The combined EtOAc extracts were dried (Na₂SO₄), filtered and concentrated to provide the title compound 37e. Yield: 2.19 g (93%). ¹H NMR (400 MHz, CDCl₃) δ=8.03 (d, 1H), 6.90 (d, 1H), 4.26 (dd, 2H), 3.80-3.72 (m, 2H), 3.40 (s, 3H), 3.18 (s, 3H), 1.85 (s, 3H).

### Step 6: N-(4-Amino-2-(2-methoxyethoxy)-5-nitrophenyl)-N-methylacetamide (37f

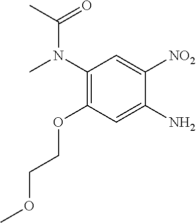 [SMILES: COCCOc1cc(N)c([N+](=O)[O-])cc1N(C)C(C)=O]

A solution of Preparation 37e (2.19 g, 7.65 mmol) in ethanol (50 mL) was treated with NH₄OH (15 mL) and the mixture was stirred at about 50° C. for about 16 h. The volatile solvents were removed on a rotary evaporator and the solids collected by filtration, rinsed with water (10 mL) and dried to provide the title compound 37f. Yield: 1.95 (90%). ¹H NMR (400 MHz, CDCl₃) δ=8.01 (s, 1H), 6.35 (br s, 2H), 6.25 (s, 1H), 4.16 (t, 2H), 3.79-3.69 (m, 2H), 3.41 (s, 3H), 3.14 (s, 3H), 1.86 (s, 3H).

### Step 7: N-(4,5-Diamino-2-(2-methoxyethoxy)phenyl)-N-methylacetamide (37q

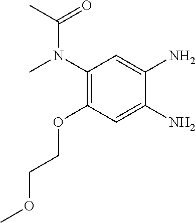 [SMILES: COCCOc1cc(N)c(N)cc1N(C)C(C)=O]

A solution of Preparation 37f (1.95 g, 6.88 mmol) in methanol (60 mL) was treated with 10% Pd/C (300 mg) and stirred under hydrogen (1 atm) at RT for about 24 h. The mixture was filtered through Celite© and rinsed with methanol (4×). The filtrate was concentrated to provide the title compound 37q. Yield: 1.74 g (99%). LC/MS m/z (M+H)⁺=254.3.

### Step 8: N-(6-(2-Methoxyethoxy)-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)-N-methylacetamide

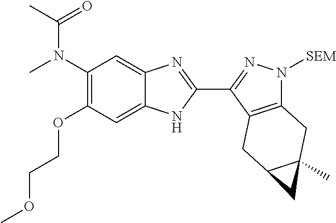

A solution of Preparation 37g (250 mg, 0.99 mmol) and Preparation 9 (302 mg, 0.98 mmol) in DMF (5 mL) was treated with sodium metabisulfite (94 mg, 0.49 mmol) and DMSO (0.18 mL, 2.47 mmol). The mixture was heated at about 110° C. for about 16 h. The reaction mixture was poured into water (20 mL) and extracted with EtOAc (3×30 mL). The combined EtOAc extracts were washed with 5% aq. LiCl and concentrated. The crude product was purified by chromatography to provide the title compound. Yield: 527 mg (99%). LC/MS m/z (M+H)⁺=540.0.

### Preparation 38: N-Methyl-N-(6-methyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)-2-(2-oxo-1,3-oxazinan-3-yl)acetamide

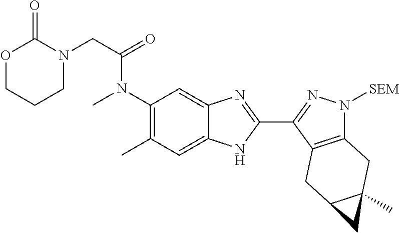 [SMILES: Cc1cc2[nH]c(-c3nn(COCC[Si](C)(C)C)c4c3C[C@@H]3C[C@]3(C)C4)nc2cc1N(C)C(=O)CN1CCCOC1=O]

### Step 1: Methyl 2-(2-oxo-1,3-oxazinan-3-yl)acetate (38a

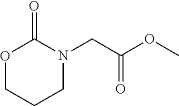

A solution of 1,3-oxazinan-2-one (800 mg, 7.91 mmol) in THF (50 mL) at about 10° C. was treated with sodium hydride (60% suspension, 506 mg, 12.7 mmol). The mixture was stirred at about 10° C. for about 30 min then treated with methyl 2-bromoacetate (0.9 mL, 9.5 mmol) dropwise. The mixture was stirred at RT for about 3 h. The mixture was treated with sat. aq. NH₄Cl (10 mL) then diluted with water (20 mL). The suspension was extracted with EtOAc (5×30 mL). The combined EtOAc extracts were dried (MgSO₄), filtered and concentrated to provide the title compound 38a which was used without additional purification. Yield: 1.5 g. ¹H NMR (400 MHz, CDCl₃) δ=4.36-4.30 (m, 2H), 4.09 (s, 2H), 3.76 (s, 3H), 3.41 (t, 2H), 2.16-2.06 (m, 2H)

### Step 2: 2-(2-Oxo-1,3-oxazinan-3-yl)acetic acid (38b

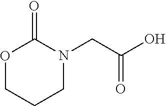 [SMILES: O=C(O)CN1CCCOC1=O]

A solution of Preparation 38a (500 mg, 2.89 mmol) in THF (45 mL) at about 10° C. was treated with methanol (15 mL), water (15 mL) and 2 N aq. NaOH (4.33 mL). The mixture was stirred at RT for about 3 h. The reaction mixture was concentrated to remove volatile solvents and the residue was acidified to ˜ pH 2 with 2 N aq. HCl. The mixture was extracted with 3:1 (v/v) chloroform/iPrOH (4×30 mL). The combined organic extracts were dried (MgSO₄), filtered and concentrated to provide the title compound 38b. Yield: 380 mg (83%). ¹H NMR (400 MHz, CD₃OD) δ=4.39-4.27 (m, 2H), 4.04 (s, 2H), 3.42 (t, 2H), 2.14-2.04 (m, 2H).

### Step 3: N-Methyl-N-(6-methyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)-2-(2-oxo-1,3-oxazinan-3-yl)acetamide

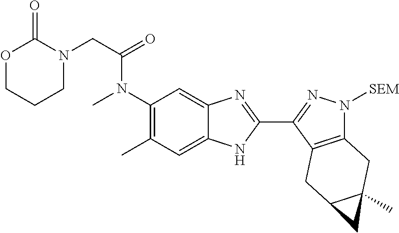 [SMILES: Cc1cc2[nH]c(-c3nn(COCC[Si](C)(C)C)c4c3C[C@@H]3C[C@]3(C)C4)nc2cc1N(C)C(=O)CN1CCCOC1=O]

A solution of Preparation 22 (50 mg, 0.11 mmol) and Preparation 38b (27 mg, 0.17 mmol) in pyridine (1.7 mL) was treated with EDCl (35 mg, 0.18 mmol) at RT. The reaction mixture was stirred at about 50° C. for about 18 h. The reaction mixture was combined with that of another identical reaction conducted using 30 mg compound 38b with similar proportions of other reagents. The combined reaction mixtures were treated with water and extracted with EtOAc (2×). The combined EtOAc extracts were dried, filtered and concentrated to provide the title compound. Yield: 120 mg. LC/MS m/z (M+H)⁺=579.2.

### Preparation 39: N-(7-Fluoro-6-methyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)-N-methylacetamide

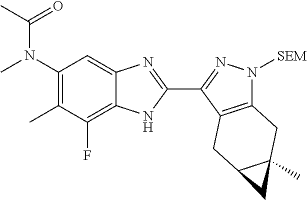

### Step 1: 3,4-Difluoro-2-methylaniline (39a

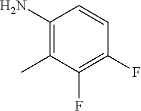

A solution of 3,4-difluoro-2-methylnitrobenzene (3.5 g, 20.2 mmol) in AcOH (100 mL) was treated with iron powder (6.77 g, 121 mmol). The mixture was stirred at RT for about 1 h. The reaction mixture was filtered and the filtrate was concentrated. The residue was taken up in EtOAc (300 mL) and washed with sat. aq. NaHCO₃ (50 mL). The EtOAc phase was dried and concentrated to provide the title compound 39a. Yield: 2.8 g, (97%). ¹H NMR (400 MHz, CDCl₃) δ=6.82 (q, 1H), 6.36 (ddd, 1H), 3.54 (br s, 2H), 2.10 (d, 3H).

### Step 2: N-(3,4-Difluoro-2-methylphenyl)acetamide (39b

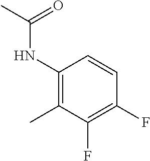

A mixture of Preparation 39a (2.80 g, 19.56 mmol) in THF (100 mL) was treated with Et₃N (5.4 mL, 39.1 mmol) and Ac₂O (3.69 mL, 39.1 mmol). The mixture was stirred at RT for about 16 h. The reaction mixture was combined with that from another identical reaction conducted using 390 mg Preparation 39a with similar proportions of other reagents. The combined reaction mixtures were concentrated and the crude product was purified by chromatography to provide the title compound 39b. Yield: 3.7 g (90%). ¹H NMR (400 MHz, CDCl₃) δ=7.36 (dt, 1H), 6.99 (q, 2H), 2.20 (s, 3H), 2.19 (d, 3H).

### Step 3: N-(3,4-Difluoro-2-methylphenyl)-N-methylacetamide (39c

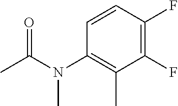

A solution of Preparation 39b (3.50 g, 18.9 mmol) in THF (100 mL) at about 0° C. was treated with sodium hydride (60% suspension, 1.51 g, 37.8 mmol) and the mixture was stirred at about 0° C. for about 30 min. The reaction mixture was treated with methyl iodide (1.76 mL, 28.4 mmol). The reaction mixture was stirred at RT for about 16 h. The reaction mixture was cooled to about 0° C. and diluted with water (10 mL). The mixture was extracted with EtOAc (2×100 mL). The combined EtOAc extracts were dried, filtered and concentrated. The crude product was purified by chromatography to provide the title compound 39c. Yield: 3.2 g (85%). ¹H NMR (400 MHz, CDCl₃) δ=7.07 (q, 1H), 6.92 (ddd, 1H), 3.16 (s, 3H), 2.20 (d, 3H), 1.78 (s, 3H).

### Step 4: N-(3,4-Difluoro-2-methyl-5-nitrophenyl)-N-methylacetamide (39d

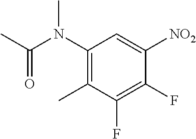 [SMILES: CC(=O)N(C)c1cc([N+](=O)[O-])c(F)c(F)c1C]

A solution of Preparation 39c (3.0 g, 15.06 mmol) in conc. H₂SO₄ (80 mL) at about 0° C. was treated with potassium nitrate (2.19 g, 22.6 mmol) in portions while keeping the internal reaction temperature below about 10° C. The mixture was stirred at about 5° C. for about 3 h. The reaction mixture was poured into ice water and extracted with EtOAc (2×200 mL). The EtOAc extracts were washed with sat. aq NaHCO₃ (3×100 mL), dried, filtered and concentrated to provide the title compound 39d. Yield: 3.40 g (92%). ¹H NMR (400 MHz, CDCl₃) δ=7.77 (dd, 1H), 3.20 (s, 3H), 2.31 (d, 3H), 1.82 (s, 3H).

### Step 5: N-(4-Amino-3-fluoro-2-methyl-5-nitrophenyl)-N-methylacetamide (39e

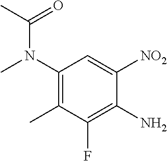 [SMILES: CC(=O)N(C)c1cc([N+](=O)[O-])c(N)c(F)c1C]

A solution of Preparation 39d (3.4 g, 13.9 mmol) in THF (50 mL) was treated with conc. NH₄OH (50 mL) and the mixture was stirred at RT for about 2 h. The reaction mixture was extracted with EtOAc (2×100 mL). The combined organic extracts were dried, filtered and concentrated to provide the title compound 39e. Yield: 3.1 g (92%). ¹H NMR (400 MHz, CDCl₃) δ=7.82 (s, 1H), 6.34-6.16 (m, 2H), 3.16 (s, 3H), 2.20 (d, 3H), 1.82 (s, 3H).

### Step 6: N-(4,5-Diamino-3-fluoro-2-methylphenyl)-N-methylacetamide (39f

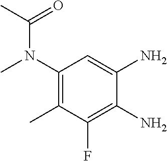

A solution of Preparation 39e (2.20 g, 9.12 mmol) in AcOH (80 mL) was treated with iron powder (3.06 g, 54.7 mmol). The mixture was stirred at RT for about 3 h. The reaction mixture was filtered and the filtrate was concentrated. The residue was taken up in EtOAc (200 mL) and washed with sat. aq. NaHCO₃ (100 mL). The EtOAc phase was dried and concentrated. The crude product was purified by chromatography to provide the title compound 39f. Yield: 1.6 g, (83%). ¹H NMR (400 MHz, CDCl₃) δ=6.33 (d, 1H), 3.46 (br s, 4H), 3.12 (s, 3H), 2.04 (d, 3H), 1.80 (s, 3H).

### Step 7: N-(7-Fluoro-6-methyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)-N-methylacetamide

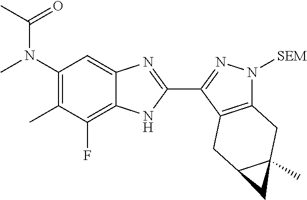

A solution of Preparation 39f (205 mg, 0.97 mmol) and Preparation 9 (297 mg, 0.97 mmol) in DMF (3 mL) and DMSO (0.17 mL, 2.43 mmol) was treated with sodium metabisulfite (92 mg, 0.49 mmol). The mixture was heated at about 110° C. for about 16 h. The reaction mixture was cooled to RT and poured into water (100 mL). The solids were collected by filtration, rinsed with water (100 mL) and dried to provide the title compound. Yield: 480 mg (99%). LC/MS m/z (M+H)⁺=498.2.

### Preparation 40: N-(6-Bromo-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)-N-methylacetamide

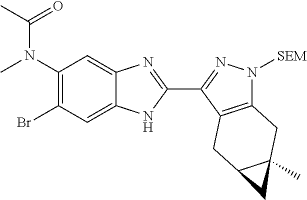

### Step 1: N-(3-Fluoro-4-nitrophenyl)acetamide (40a

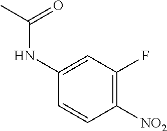

A mixture of 3-fluoro-4-nitroaniline (25 g, 160 mmol) in Ac₂O (300 mL) was stirred at about 15° C. for about 16 h. The reaction mixture was treated with water (100 mL) and the resultant solids were collected, rinsed with water (2×50 mL) and dried to provide the title compound 40a. Yield: 25 g (79%). ¹H NMR (400 MHz, CDCl₃) δ=8.09 (t, 1H), 7.86 (dd, 1H), 7.38 (td, 1H), 2.17 (s, 3H).

### Step 2: N-(3-Fluoro-4-nitrophenyl)acetamide (40b

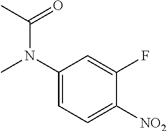

A solution of Preparation 40a (23 g, 116 mmol) in THF (300 mL) was treated with KOtBu (14.3 g, 128 mmol) in portions at about 0° C. The mixture was stirred at about 0° C. for 1 h. A solution of methyl iodide (7.94 mL, 128 mmol) in THF (80 mL) was added dropwise at about 0° C. The reaction mixture was stirred at about 15° C. for 16 h. The reaction mixture was treated with sat. aq. NH₄Cl (120 mL) and extracted with EtOAc (2×100 mL). The combined EtOAc extracts were washed with brine, dried (Na₂SO₄), filtered and concentrated. The crude product was purified by chromatography to provide the title compound 40b. Yield: 22 g (89%). ¹H NMR (400 MHz, CDCl₃) δ=8.14 (t, 1H), 7.26-7.17 (m, 2H), 3.36 (s, 3H), 2.11 (s, 3H).

### Step 3: N-(3-Amino-4-nitrophenyl)-N-methylacetamide (40c

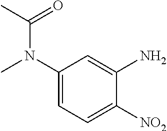

A solution of Preparation 40b (22.0 g, 103.7 mmol) in ethanol (400 mL) was treated with conc. NH₄OH (160 mL) and the mixture was stirred at about 50° C. for about 16 h. Additional NH₄OH (80 mL) was added and the mixture was stirred at about 60° C. for about 16 h. The reaction mixture was combined with that from another identical reaction conducted using 1.51 g of compound 40b and similar proportions of other reagents. The combined reaction mixtures were concentrated and the residue was triturated with MTBE (200 mL). The solids were collected by filtration and dried to provide the title compound 40c. Yield: 20.5 g (88%). ¹H NMR (400 MHz, CDCl₃) δ=8.18 (d, 1H), 6.68 (d, 1H), 6.55 (dd, 1H), 6.23 (br s, 2H), 3.27 (s, 3H), 2.03 (s, 3H).

### Step 4: N-(5-Amino-2-bromo-4-nitrophenyl)-N-methylacetamide (40d

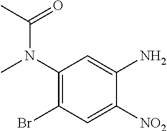 [SMILES: CC(=O)N(C)c1cc(N)c([N+](=O)[O-])cc1Br]

A solution of Preparation 40c (1.0 g, 4.78 mmol) in DMF (25 mL) was treated with N-bromo succinimide (1.11 g, 6.21 mmol) at about 0° C. The reaction mixture was stirred at about 0° C. for about 30 min and then at RT for about 1 h. The reaction mixture was diluted with water (50 mL) and extracted with EtOAc (150 mL and 50 m). The combined EtOAc extracts were washed with brine, dried, filtered and concentrated. The crude product was purified by chromatography to provide the title compound 40d. Yield: 1.05 g (76%). LC/MS m/z (M+H)⁺=289.9 (⁸¹Br).

### Step 5: N-(4,5-diamino-2-bromophenyl)-N-methylacetamide (40e

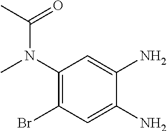

A solution of Preparation 40d (1.0 g, 3.47 mmol) in ethanol (50 mL) was treated with sat. aq. NH₄Cl (3 mL) and iron powder (582 mg, 10.4 mmol). The mixture was stirred at about 70° C. for about 1 h. The reaction mixture was filtered and the filtrate was concentrated. The crude product was purified by chromatography to provide the title compound 40e. Yield: 850 mg, (95%). LC/MS m/z (M+H)=259.9 (⁸¹Br).

### Step 6: N-(7-Fluoro-6-methyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)-N-methylacetamide

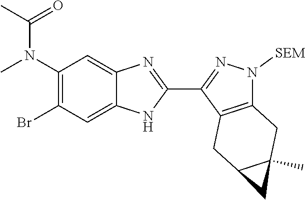

A solution of Preparation 40e (150 mg, 0.58 mmol) in DMF (3 mL) was treated with Preparation 9 (178 mg, 0.58 mmol) and sodium metabisulfite (221 mg, 1.16 mmol). The mixture was heated in a microwave reactor at about 150° C. for about 2 h. The reaction mixture was cooled to RT and poured into water (20 mL) and extracted with EtOAc (2×50 mL). The combined EtOAc extracts were washed with brine, dried, filtered and concentrated. The crude product was purified by prep-HPLC (Boston Prime C18 30 mm×150 mm, 5 μm; Mobile phase A: water (0.05% NH₄OH); Mobile phase B: MeCN; 70-90% B gradient; 10 min, 25 mL/min) to provide the title compound. Yield: 161 mg (80%). LC/MS m/z (M+H)⁺=546.1 (⁸¹Br).

### Preparation 41: N-(7-Fluoro-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)-N-methyl-2-(2-oxo-1,3-oxazinan-3-yl)propenamide

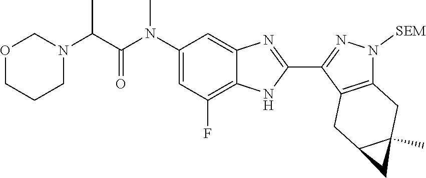 [SMILES: CC(C(=O)N(C)c1cc(F)c2[nH]c(-c3nn(COCC[Si](C)(C)C)c4c3C[C@@H]3C[C@]3(C)C4)nc2c1)N1CCCOC1]

### Step 1: Benzyl 2-bromopropanoate (41a

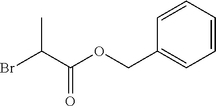

A solution of 2-bromopropionic acid (5.0 g, 32.7 mmol) and Et₃N (5.0 mL, 36.0 mmol) in DCM (100 mL) at about 0° C. was treated with benzyl chloroformate (4.67 mL, 32.7 mmol) dropwise. The mixture was stirred at about 0° C. for about 10 min and then treated with DMAP (399 mg, 3.3 mmol). The mixture was stirred at about 0° C. for about 30 min and then stirred at about 30° C. for about 4 h. The reaction mixture was diluted with 1 N aq. HCl (15 mL) and brine (80 mL) and then extracted with DCM (2×80 mL). The combined DCM extracts were concentrated and purified by chromatography to provide the title compound 41a. Yield: 4.81 g, 86%). ¹H NMR (400 MHz, CDCl₃) δ=7.42-7.33 (m, 5H), 5.29-5.16 (m, 2H), 4.43 (q, 1H), 1.85 (d, 3H).

### Step 2: Benzyl 2-(2-oxo-1,3-oxazinan-3-yl)propanoate (41b

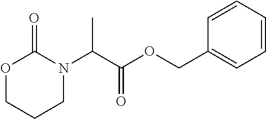 [SMILES: CC(C(=O)OCc1ccccc1)N1CCCOC1=O]

A solution of 1,3-oxazinan-2-one (800 mg, 7.91 mmol) in THF (53 mL) at about 10° C. was treated with sodium hydride (60% suspension, 506 mg, 12.7 mmol) and the mixture was stirred at about 10° C. for about 30 min. The reaction mixture was treated with Preparation 41a (2.13 g, 9.50 mmol) at about 10° C. and stirred at RT for about 3 h. The reaction mixture was treated with sat. aq. NH₄Cl (50 mL) and diluted with water (40 mL). The mixture was extracted with EtOAc (30 mL). The EtOAc extract was dried (MgSO₄), filtered and concentrated. The crude product was purified by chromatography to provide the title compound 41b. Yield: 371 mg (18%). ¹H NMR (400 MHz, CDCl₃) δ=7.43-7.29 (m, 5H), 5.25-5.09 (m, 2H), 5.02 (q, 1H), 4.36-4.15 (m, 2H), 3.36-3.22 (m, 2H), 2.10-1.94 (m, 2H), 1.47 (d, 3H).

### Step 3: Benzyl 2-(2-oxo-1,3-oxazinan-3-yl)propanoate (41c

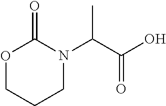

A solution of Preparation 41b (371 mg, 1.41 mmol) in methanol (16 mL) was treated with 10% Pd/C (300 mg, 50% water) and stirred under hydrogen (1 atm) at RT for about 16 h. The mixture was filtered through Celite© and rinsed with methanol. The filtrate was concentrated to provide the title compound 41c. Yield: 259 mg. ¹H NMR (400 MHz, CDCl₃) δ=4.63 (br d, 1H), 4.35-4.15 (m, 2H), 3.44-3.24 (m, 2H), 2.21-1.91 (m, 2H), 1.40 (br d, 3H).

### Step 4: N-(7-fluoro-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)-N-methyl-2-(2-oxo-1,3-oxazinan-3-yl)propanamide

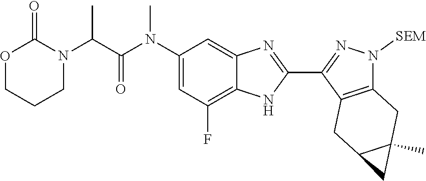 [SMILES: CC(C(=O)N(C)c1cc(F)c2[nH]c(-c3nn(COCC[Si](C)(C)C)c4c3C[C@@H]3C[C@]3(C)C4)nc2c1)N1CCCOC1=O]

A solution of Preparation 32 (350 mg, 0.79 mmol) and Preparation 41c (137 mg, 0.79 mmol) in pyridine (11.3 mL) was treated with EDCl (304 mg, 1.59 mmol) at RT. The reaction mixture was stirred at RT for about 16 h. The reaction mixture was combined with that of another identical reaction conducted using 50 mg of compound 32 and similar proportions of other reagents. The combined reaction mixtures were treated with water (5 mL) and concentrated. The crude product was purified by chromatography to provide the title compound. Yield: 250 mg (46%). LC/MS m/z (M+H)⁺=597.2.

### Preparation 42: 4-Fluoro-N-methyl-N-(5-methyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-6-yl)tetrahydro-2H-pyran-4-carboxamide

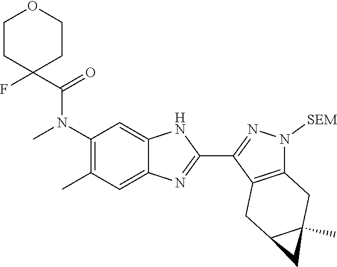 [SMILES: Cc1cc2nc(-c3nn(COCC[Si](C)(C)C)c4c3C[C@@H]3C[C@]3(C)C4)[nH]c2cc1N(C)C(=O)C1(F)CCOCC1]

A solution of Preparation 22 (100 mg, 0.23 mmol) in pyridine (5 mL) was treated with EDCl (88 mg, 0.46 mmol) and 4-fluorotetrahydro-2H-pyran-4-carboxylic acid (CAS: 1150617-62-1, 34 mg, 0.23 mmol). The reaction mixture was stirred at RT for about 16 h. The reaction mixture was treated with water (10 mL) and extracted with EtOAc (10 mL). The EtOAc extract was dried (Na₂SO₄), filtered and concentrated to provide the title compound. Yield: 160 mg. LC/MS m/z (M+H)⁺=568.3.

### Preparation 43: (1R,5S)-6,6-Difluoro-N-methyl-N-(5-methyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-6-yl)bicyclo[3.1.0]hexane-3-carboxamide

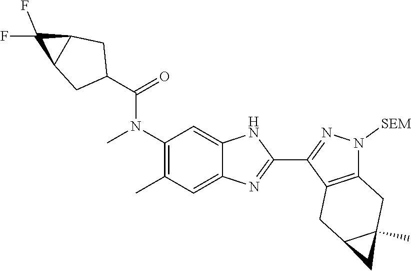 [SMILES: Cc1cc2nc(-c3nn(COCC[Si](C)(C)C)c4c3C[C@@H]3C[C@]3(C)C4)[nH]c2cc1N(C)C(=O)C1C[C@@H]2[C@H](C1)C2(F)F]

A solution of Preparation 22 (100 mg, 0.23 mmol) in pyridine (5 mL) was treated with EDCl (88 mg, 0.46 mmol) and (1R,5S)-6,6-difluorobicyclo[3.1.0]hexane-3-carboxylic acid (MFCD29920567, 37 mg, 0.23 mmol). The reaction mixture was stirred at RT for about 16 h. The reaction mixture was combined with that of another identical reaction conducted using 20 mg of Preparation 22 and similar proportions of other reagents. The combined reaction mixtures were treated with water (15 mL) and extracted with EtOAc (20 mL). The EtOAc extract was dried (Na₂SO₄), filtered and concentrated to provide the title compound. Yield: 190 mg. LC/MS m/z (M+H)⁺=582.3.

### Preparation 44: N-Methyl-N-(6-methyl-2-((4aS,5aR)-1-((2-(trimethylsilyl)ethoxy)methyl)-1,4,4a,5,5a,6-hexahydrocyclopropa[f]indazol-3-yl)-1H-benzo[d]imidazol-5-yl)-2-(piperidin-1-yl)propanamide

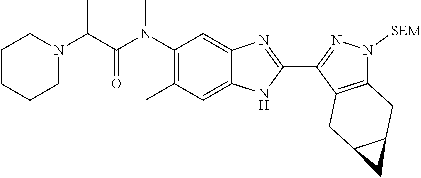 [SMILES: Cc1cc2[nH]c(-c3nn(COCC[Si](C)(C)C)c4c3C[C@@H]3C[C@@H]3C4)nc2cc1N(C)C(=O)C(C)N1CCCCC1]

### Step 1: Benzyl 2-(piperidin-1-yl)propanoate (44a

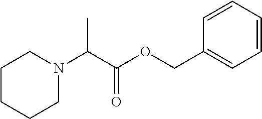 [SMILES: CC(C(=O)OCc1ccccc1)N1CCCCC1]

A solution of piperidine (300 mg, 3.52 mmol) in MeCN (15 mL) was treated with Preparation 41a (942 mg, 3.88 mmol) and NaHCO₃ (444 mg, 5.28 mmol). The reaction mixture was stirred at RT for about 3 h. The reaction mixture was filtered and the filtrate was concentrated. The crude product was purified by chromatography to provide the title compound 44a. Yield: 450 mg (52%). ¹H NMR (400 MHz, CDCl₃) δ=7.44-7.30 (m, 5H), 5.16 (d, 2H), 3.33 (q, 1H), 2.64-2.44 (m, 4H), 1.65-1.51 (m, 4H), 1.47-1.37 (m, 2H), 1.31 (d, 3H).

### Step 2: 2-(Piperidin-1-yl)propanoic acid (44b

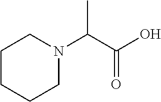

A solution of Preparation 44a (344 mg, 1.39 mmol) in methanol (10 mL) was treated with 10% Pd/C (296 mg, 50% water). The mixture was stirred under hydrogen (1 atm) at RT for about 16 h. The mixture was filtered through Celite© and rinsed with methanol (3×20 mL). The filtrate was concentrated to provide the title compound 44b. Yield: 152 mg (70%). ¹H NMR (400 MHz, CD₃OD) δ=3.53 (q, 1H), 3.37-3.07 (br m, 4H), 1.96-1.80 (m, 4H), 1.74-1.57 (m, 2H), 1.49 (d, 3H).

### Step 3: N-Methyl-N-(6-methyl-2-((4aS,5aR)-1-((2-(trimethylsilyl)ethoxy)methyl)-1,4,4a,5,5a,6-hexahydrocyclopropa[f]indazol-3-yl)-1H-benzo[d]imidazol-5-yl)-2-(piperidin-1-yl)propanamide

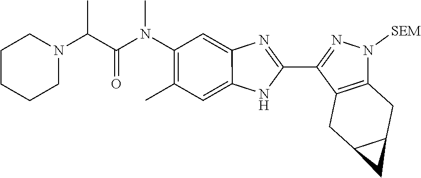 [SMILES: Cc1cc2[nH]c(-c3nn(COCC[Si](C)(C)C)c4c3C[C@@H]3C[C@@H]3C4)nc2cc1N(C)C(=O)C(C)N1CCCCC1]

A solution of Preparation 44b (120 mg, 0.76 mmol) in DMF (15 mL) was treated with HATU (435 mg, 1.14 mmol), Preparation 22 (334 mg, 0.76 mmol) and iPr₂NEt (0.26 mL, 1.53 mmol). The reaction mixture was stirred at about 60° C. for about 16 h. The reaction mixture was treated with 3% aq. LiCl (50 mL) and extracted with EtOAc (3×5 mL). The combine EtOAc extracts were washed with brine (10 mL) and concentrated. The crude product was purified by chromatography to provide the title compound. Yield: 150 mg (34%). LC/MS m/z (M+H)⁺=577.3.

### Preparation 45 N-Methyl-N-(6-methyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)-2-(1,4-oxazepan-4-yl)propanamide (45)

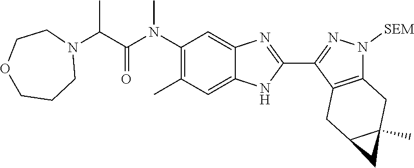 [SMILES: Cc1cc2[nH]c(-c3nn(COCC[Si](C)(C)C)c4c3C[C@@H]3C[C@]3(C)C4)nc2cc1N(C)C(=O)C(C)N1CCCOCC1]

### Step 1: Benzyl 2-(1,4-oxazepan-4-yl)propanoate (45a

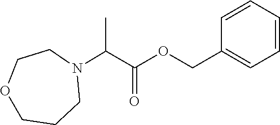 [SMILES: CC(C(=O)OCc1ccccc1)N1CCCOCC1]

A suspension of 1,4-oxazepane (458 mg, 4.52 mmol) in MeCN (20 mL) was treated with Preparation 41a (1.00 g, 4.11 mmol) and NaHCO₃ (518 mg, 6.17 mmol). The reaction mixture was stirred at RT for about 20 h and then at about 50° C. for about 16 h. The reaction mixture was filtered and the filtrate was concentrated. The crude product was purified by chromatography to provide the title compound 45a. Yield: 560 mg (52%). ¹H NMR (400 MHz, CDCl₃) δ=7.45-7.30 (m, 5H), 5.15 (d, 2H), 3.78 (t, 2H), 3.75-3.61 (m, 2H), 3.54 (q, 1H), 2.95-2.87 (m, 2H), 2.84-2.73 (m, 2H), 1.94-1.75 (m, 2H), 1.33 (d, 3H).

### Step 2: 2-(1,4-Oxazepan-4-yl)propanoic acid (45b

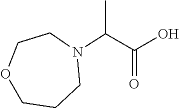

A solution of Preparation 45a (460 mg, 1.75 mmol) in methanol (20 mL) was treated with 10% Pd/C (300 mg, 50% water). The mixture was stirred under hydrogen (1 atm) at RT for about 16 h. The mixture was filtered through Celite© and the filter was washed with methanol (3×50 mL). The filtrate was concentrated to provide the title compound 45b. Yield: 280 mg (93%). ¹H NMR (400 MHz, CD₃OD) δ=3.95-3.90 (m, 2H), 3.87-3.80 (m, 2H), 3.75 (q, 1H), 3.51-3.33 (m, 4H), 2.16 (quin, 2H), 1.52 (d, 3H).

### Step 3: N-Methyl-N-(6-methyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)-2-(1,4-oxazepan-4-yl)propanamide

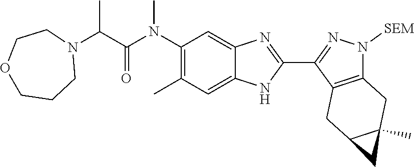 [SMILES: Cc1cc2[nH]c(-c3nn(COCC[Si](C)(C)C)c4c3C[C@@H]3C[C@]3(C)C4)nc2cc1N(C)C(=O)C(C)N1CCCOCC1]

A solution of Preparation 45b (256 mg, 1.48 mmol) in DMF (30 mL) was treated with HATU (843 mg, 2.22 mmol), Preparation 22 (647 mg, 1.48 mmol) and iPr₂NEt (0.51 mL, 2.96 mmol). The reaction mixture was stirred at about 60° C. for about 16 h. The reaction mixture was treated with 3% aq. LiCl (50 mL) and extracted with EtOAc (3×5 mL). The combine EtOAc extracts were washed with brine and concentrated. The crude product was purified by chromatography to provide the title compound. Yield: 90 mg (10%). LC/MS m/z (M+H)⁺=593.3.

### Preparation 46: N-(6-Ethyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)-N-methylacetamide

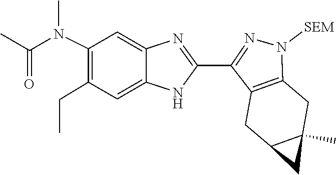 [SMILES: CCc1cc2[nH]c(-c3nn(COCC[Si](C)(C)C)c4c3C[C@@H]3C[C@]3(C)C4)nc2cc1N(C)C(C)=O]

### Step 1: N-(5-Chloro-2-ethylphenyl)acetamide (46a

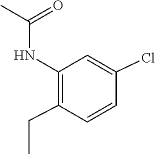

5-Chloro-2-ethylaniline (1.0 g, 6.43 mmol) was added to Ac₂O (30 mL) with stirring at about 15° C. The reaction mixture was stirred at about 15° C. for about 16 h and filtered to collect the precipitate. The solids were washed with water (3×15 mL) and dried. The filtrate was extracted with EtOAc (30 mL). The EtOAc extract was washed with sat. aq Na₂CO₃ (2×60 mL) and water (60 mL). The EtOAc extract was dried, filtered and concentrated. The residue was combined with the previously isolated solids to provide the title compound 46a. Yield: 920 mg (72%). ¹H NMR (400 MHz, CDCl₃) δ 7.91 (s, 1H), 7.12 (t, 2H), 6.96 (s, 1H), 2.56 (q, 2H), 2.21 (s, 3H), 1.22 (t, 3H); LC/MS m/z (M+H) 10=197.9.

### Step 2: N-(5-Chloro-2-ethylphenyl)-N-methylacetamide (46b

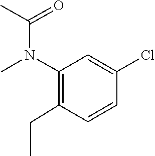

A solution of Preparation 46a (1.14 g, 5.77 mmol) in THF (55 mL) at about 0° C. was treated with sodium hydride (60% suspension, 350 mg, 8.80 mmol) and the mixture was stirred at about 15° C. for about 30 min. The reaction mixture was treated with methyl iodide (0.4 mL, 6.5 mmol). The reaction mixture was stirred about 15° C. for about 18 h. The reaction mixture was treated with sat. aq. NH₄Cl (50 mL) and extracted with EtOAc (2×50 mL). The combined EtOAc extracts were concentrated. The residue was combined with that of another identical reaction conducted using 100 mg Preparation 46a and similar proportions of other reagents. The combined residues were purified by chromatography to provide the title compound 46b. Yield: 1.29 g (97%). ¹H NMR (400 MHz, CDCl₃) δ=7.34-7.27 (m, 2H), 7.14 (d, 1H), 3.18 (s, 3H), 2.54 (dq, 2H), 1.80 (s, 3H), 1.23 (t, 3H).

### Step 3: N-(5-Chloro-2-ethyl-4-nitrophenyl)-N-methylacetamide (46c

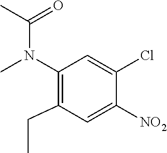 [SMILES: CCc1cc([N+](=O)[O-])c(Cl)cc1N(C)C(C)=O]

A solution of Preparation 46b (1.09 g, 5.17 mmol) in conc. H₂SO₄ (8.2 mL) at about 0° C. was treated with potassium nitrate (522 mg, 5.17 mmol) added in portions while keeping the internal reaction temperature below about 5° C. The mixture was stirred at about 0° C. for about 2 h. The reaction mixture was poured into ice water (50 mL) and stirred for about 10 min. The solids were collected by filtration, washed with water (3×10 mL) and dried. The crude product was purified by chromatography to provide the title compound 46c. Yield: 1.07 g (80%). ¹H NMR (400 MHz, CDCl₃) δ=7.89 (s, 1H), 7.36 (s, 1H), 3.20 (s, 3H), 2.63 (dq, 2H), 1.83 (s, 3H), 1.29 (t, 3H).

### Step 4: N-(5-(Benzylamino)-2-ethyl-4-nitrophenyl)-N-methylacetamide (46d

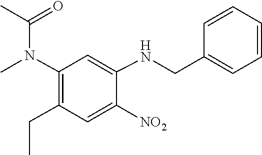 [SMILES: CCc1cc([N+](=O)[O-])c(NCc2ccccc2)cc1N(C)C(C)=O]

A mixture of Preparation 46c (965 mg, 3.76 mmol) and benzylamine (2.5 mL, 22.6 mmol) was treated with ammonium acetate (290 mg, 3.76 mmol) at about 15° C. The mixture was stirred at about 100° C. for about 16 h. The reaction mixture was combined with that of another identical reaction conducted using 100 mg compound 46c and similar proportions of other reagents. This mixture was diluted with EtOAc and washed with 3 N aq. HCl (2×30 mL) and brine (40 mL). The EtOAc layer was concentrated and the residue was purified by chromatography to provide the title compound 46d. Yield: 1.33 g (98%). ¹H NMR (400 MHz, CDCl₃) δ=8.39-8.30 (m, 1H), 8.18 (s, 1H), 7.40-7.28 (m, 7H), 6.52 (s, 1H), 4.62-4.47 (m, 2H), 3.09 (s, 3H), 2.46 (q, 2H), 1.63 (s, 3H), 1.23 (t, 3H).

### Step 5: N-(4,5-Diamino-2-ethylphenyl)-N-methylacetamide (46e

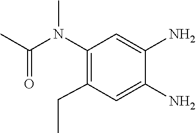

A suspension of Preparation 46d (1.33 g, 4.08 mmol) and 10% Pd/C (140 mg) in methanol (10 mL) was stirred under hydrogen (3 atm) at RT for about 24 h. Additional 10% Pd/C (200 mg) was added and the mixture stirred under hydrogen (3 atm) for about 72 h. The mixture was filtered through Celite©, the filter was washed with methanol and the filtrate was concentrated. The crude product was purified by chromatography to provide the title compound 46e. Yield: 665 mg (79%). ¹H NMR (400 MHz, CDCl₃) δ=6.62 (s, 1H), 6.45 (s, 1H), 3.48-3.38 (m, 2H), 3.58-3.28 (m, 2H), 3.15 (s, 3H), 2.47-2.35 (m, 2H), 1.80 (s, 3H), 1.17 (t, 3H).

### Step 5: N-(6-Ethyl-2-((4aS,5aR)-5a-methyl-1-((2-(trimethylsilyl)ethoxy)methyl)-1,4,4a,5,5a,6-hexahydrocyclopropa[f]indazol-3-yl)-1H-benzo[d]imidazol-5-yl)-N-methylacetamide

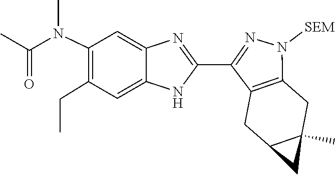 [SMILES: CCc1cc2[nH]c(-c3nn(COCC[Si](C)(C)C)c4c3C[C@@H]3C[C@]3(C)C4)nc2cc1N(C)C(C)=O]

A solution of Preparation 46e (665 mg, 3.21 mmol) and sodium metabisulfite (305 mg, 1.60 mmol) in DMF (17 mL) was treated with Preparation 9 (983 mg, 3.21 mmol) and DMSO (0.57 mL, 8.02 mmol). The mixture was stirred at about 110° C. for about 16 h. The reaction mixture was poured into water (30 mL). The solids were collected by filtration and dried to provide the title compound. Yield: 1.18 g (75%). LC/MS m/z (M+H)⁺=493.9.

### EXAMPLES

### Example 1: N-Methyl-N-(6-methyl-2-((4aS,5aR)-5a-methyl-1,4,4a,5,5a,6-hexahydrocyclopropa[f]indazol-3-yl)-1H-benzo[d]imidazol-5-yl)-2-(2-oxo-1,3-oxazinan-3-yl)acetamide

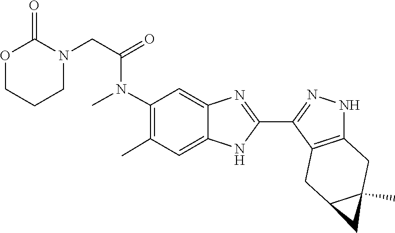 [SMILES: Cc1cc2[nH]c(-c3n[nH]c4c3C[C@@H]3C[C@]3(C)C4)nc2cc1N(C)C(=O)CN1CCCOC1=O]

A solution of Preparation 38 (120 mg, 0.21 mmol) in TFA (2 mL) at about 0° C. was treated with triethylsilane (0.17 mL, 1.04 mmol). The mixture was stirred at RT for about 2 h. The reaction mixture was concentrated and the residue was treated with sat. aq. NaHCO₃ and extracted with DCM (3×8 mL). The combined DCM extracts were concentrated and the crude product was purified by prep-HPLC (YMC Actus Triart C18 150 mm×30 mm, 5 μm; Mobile phase A: water (0.05% conc. NH₄OH); Mobile phase B: MeCN; Gradient: 33-53% B over 10 min, hold at 100% B for 2 min.; flow rate=35 mL/min) to provide the title compound. Yield: 40 mg (43%). ¹H NMR (400 MHz, CD₃OD) δ=7.71-7.40 (m, 2H), 4.35-4.26 (m, 2H), 3.75-3.68 (m, 1H), 3.44-3.33 (m, 2H), 3.26 (s, 3H), 3.17-3.10 (m, 1H), 3.06 (d, 1H), 2.77 (d, 1H), 2.40 (s, 3H), 2.13-1.97 (m, 2H), 1.29 (s, 3H), 1.19-1.11 (m, 1H), 0.41 (dd, 1H), 0.25 (br t, 1H); LC/MS m/z (M+H)+=449.1.

Examples 2 to 16 in the table below were prepared in a similar fashion to Example 1, from the appropriate precursor Preparation (shown in parenthesis), using similar proportions of reagents. Observed LC-MS m/z is shown in the table as [M+H]⁺.

| Ex | Structure and Name (Precursor Preparation No.) | [M + H]⁺ |
|---|---|---|
| 2 | 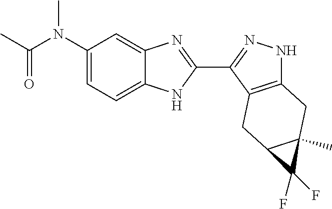 [SMILES: CC(=O)N(C)c1ccc2[nH]c(-c3n[nH]c4c3C[C@@H]3C(F)(F)[C@]3(C)C4)nc2c1] | 372.3 |
| | N-(2-((4aS,5aR)-5,5-difluoro-5a-methyl-1,4,4a,5,5a,6- | |
| | hexahydrocyclopropa[f]indazol-3-yl)-1H-benzo[d]imidazol-5-yl)-N- | |
| | methylacetamide (Preparation 33) | |
| 3 | 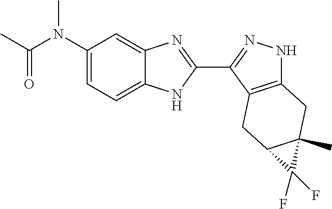 [SMILES: CC(=O)N(C)c1ccc2[nH]c(-c3n[nH]c4c3C[C@H]3C(F)(F)[C@@]3(C)C4)nc2c1] | 372.3 |
| | N-(2-((4aR,5aS)-5,5-difluoro-5a-methyl-1,4,4a,5,5a,6- | |
| | hexahydrocyclopropa[f]indazol-3-yl)-1H-benzo[d]imidazol-5-yl)-N- | |
| | methylacetamide (Preparation 33) | |
| 4 | 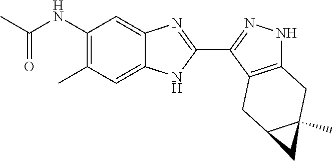 [SMILES: CC(=O)Nc1cc2nc(-c3n[nH]c4c3C[C@@H]3C[C@]3(C)C4)[nH]c2cc1C] | 336.3 |
| | N-(6-methyl-2-((4aS,5aR)-5a-methyl-1,4,4a,5,5a,6- | |
| | hexahydrocyclopropa[f]indazol-3-yl)-1H-benzo[d]imidazol-5- | |
| | yl)acetamide (Preparation 34) | |
| 5 | 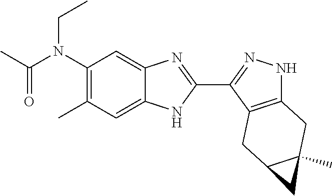 [SMILES: CCN(C(C)=O)c1cc2nc(-c3n[nH]c4c3C[C@@H]3C[C@]3(C)C4)[nH]c2cc1C] | 364.3 |
| | N-ethyl-N-(6-methyl-2-((4aS,5aR)-5a-methyl-1,4,4a,5,5a,6- | |
| | hexahydrocyclopropa[f]indazol-3-yl)-1H-benzo[d]imidazol-5- | |
| | yl)acetamide (Preparation 35) | |
| 6 | 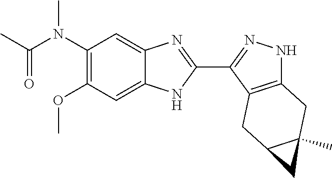 [SMILES: COc1cc2[nH]c(-c3n[nH]c4c3C[C@@H]3C[C@]3(C)C4)nc2cc1N(C)C(C)=O] | 366.1 |
| | N-(6-methoxy-2-((4aS,5aR)-5a-methyl-1,4,4a,5,5a,6- | |
| | hexahydrocyclopropa[f]indazol-3-yl)-1H-benzo[d]imidazol-5-yl)-N- | |
| | methylacetamide (Preparation 36) | |
| 7 | 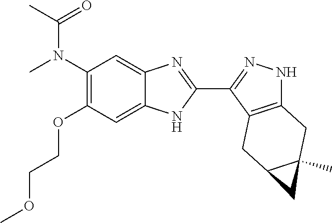 | 410.3 |
| | N-(6-(2-methoxyethoxy)-2-((4aS,5aR)-5a-methyl-1,4,4a,5,5a,6- | |
| | hexahydrocyclopropa[f]indazol-3-yl)-1H-benzo[d]imidazol-5-yl)-N- | |
| | methylacetamide (Preparation 37) | |
| 8 | 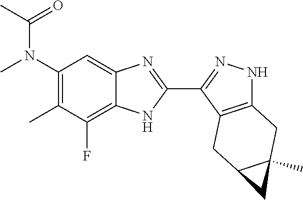 | 368.3 |
| | N-(7-fluoro-6-methyl-2-((4aS,5aR)-5a-methyl-1,4,4a,5,5a,6- | |
| | hexahydrocyclopropa[f]indazol-3-yl)-1H-benzo[d]imidazol-5-yl)-N- | |
| | methylacetamide (Preparation 39) | |
| 9 | 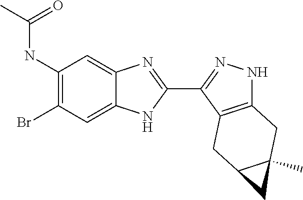 [SMILES: CC(=O)Nc1cc2nc(-c3n[nH]c4c3C[C@@H]3C[C@]3(C)C4)[nH]c2cc1Br] | 414.2 |
| | N-(6-bromo-2-((4aS,5aR)-5a-methyl-1,4,4a,5,5a,6- | |
| | hexahydrocyclopropa[f]indazol-3-yl)-1H-benzo[d]imidazol-5-yl)-N- | |
| | methylacetamide (Preparation 40) | |
| 10 | 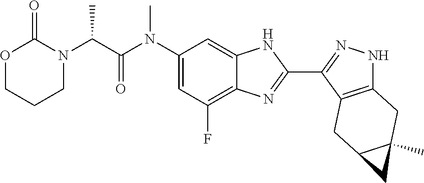 [SMILES: C[C@H](C(=O)N(C)c1cc(F)c2nc(-c3n[nH]c4c3C[C@@H]3C[C@]3(C)C4)[nH]c2c1)N1CCCOC1=O] | 467.3 |
| | (R)-N-(4-fluoro-2-((4aS,5aR)-5a-methyl-1,4,4a,5,5a,6- | |
| | hexahydrocyclopropa[f]indazol-3-yl)-1H-benzo[d]imidazol-6-yl)-N- | |
| | methyl-2-(2-oxo-1,3-oxazinan-3-yl)propenamide (Preparation 41) | |
| 11 | 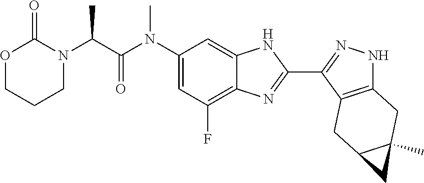 [SMILES: C[C@@H](C(=O)N(C)c1cc(F)c2nc(-c3n[nH]c4c3C[C@@H]3C[C@]3(C)C4)[nH]c2c1)N1CCCOC1=O] | 467.2 |
| | (S)-N-(4-fluoro-2-((4aS,5aR)-5a-methyl-1,4,4a,5,5a,6- | |
| | hexahydrocyclopropa[f]indazol-3-yl)-1H-benzo[d]imidazol-6-yl)-N- | |
| | methyl-2-(2-oxo-1,3-oxazinan-3-yl)propenamide (Preparation 41) | |
| 12 | 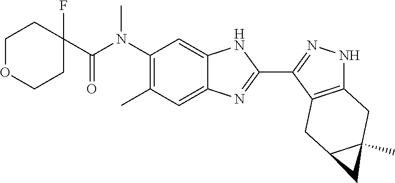 [SMILES: Cc1cc2nc(-c3n[nH]c4c3C[C@@H]3C[C@]3(C)C4)[nH]c2cc1N(C)C(=O)C1(F)CCOCC1] | 438.2 |
| | 4-fluoro-N-methyl-N-(5-methyl-2-((4aS,5aR)-5a-methyl-1,4,4a,5,5a,6- | |
| | hexahydrocyclopropa[f]indazol-3-yl)-1H-benzo[d]imidazol-6- | |
| | yl)tetrahydro-2H-pyran-4-carboxamide (Preparation 42) | |
| 13 | 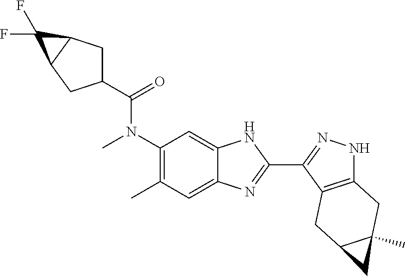 [SMILES: Cc1cc2nc(-c3n[nH]c4c3C[C@@H]3C[C@]3(C)C4)[nH]c2cc1N(C)C(=O)C1C[C@@H]2[C@H](C1)C2(F)F] | 452.2 |
| | (1R,5S)-6,6-difluoro-N-methyl-N-(5-methyl-2-((4aS,5aR)-5a-methyl- | |
| | 1,4,4a,5,5a,6-hexahydrocyclopropa[f]indazol-3-yl)-1H- | |
| | benzo[d]imidazol-6-yl)bicyclo[3.1.0]hexane-3-carboxamide (Prep'n 43) | |
| 14 | 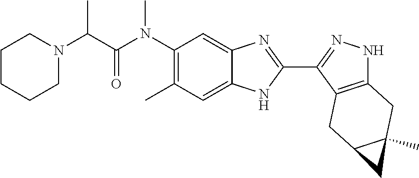 [SMILES: Cc1cc2[nH]c(-c3n[nH]c4c3C[C@@H]3C[C@]3(C)C4)nc2cc1N(C)C(=O)C(C)N1CCCCC1] | 447.3 |
| | N-methyl-N-(6-methyl-2-((4aS,5aR)-5a-methyl-1,4,4a,5,5a,6- | |
| | hexahydrocyclopropa[f]indazol-3-yl)-1H-benzo[d]imidazol-5-yl)-2- | |
| | (piperidin-1-yl)propenamide (Preparation 44) | |
| 15 | 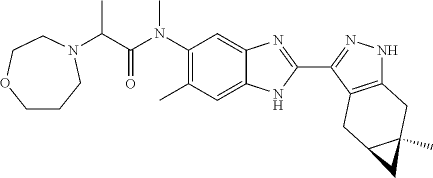 [SMILES: Cc1cc2[nH]c(-c3n[nH]c4c3C[C@@H]3C[C@]3(C)C4)nc2cc1N(C)C(=O)C(C)N1CCCOCC1] | 463.4 |
| | N-methyl-N-(6-methyl-2-((4aS,5aR)-5a-methyl-1,4,4a,5,5a,6- | |
| | hexahydrocyclopropa[f]indazol-3-yl)-1H-benzo[d]imidazol-5-yl)-2-(1,4- | |
| | oxazepan-4-yl)propenamide (Preparation 45) | |
| 16 | 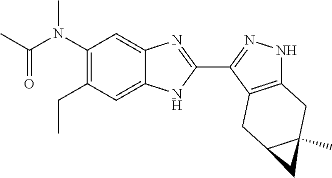 [SMILES: CCc1cc2[nH]c(-c3n[nH]c4c3C[C@@H]3C[C@]3(C)C4)nc2cc1N(C)C(C)=O] | 364.2 |
| | N-(6-ethyl-2-((4aS,5aR)-5a-methyl-1,4,4a,5,5a,6- | |
| | hexahydrocyclopropa[f]indazol-3-yl)-1H-benzo[d]imidazol-5-yl)-N- | |
| | methylacetamide (Preparation 46) | |

Examples 17 to 40 in the table below were prepared by parallel synthesis according to the scheme and reaction procedure that follows:

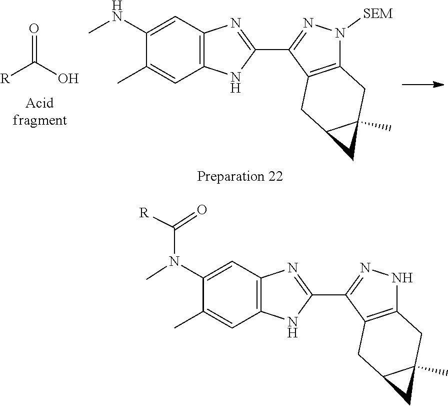

Each 2-dram vial was charged with acid fragment (0.1 mmol) and Preparation 22 (0.15 mmol) followed by addition of pyridine (34 μL, 0.45 mmol), THF (0.11 mL) and T3P solution (50 wt % solution in EtOAc, 0.2 mmol). The vials were heated to about 80° C. and shaken for about 16 h. The solvents were removed under reduced pressure. The residue was treated with sat. aq. NaHCO₃ and extracted with EtOAc (3×). The combined EtOAc extracts were dried (Na₂SO₄), filtered and concentrated. The residue in each vial was suspended in 5:1 (v/v) DCM/TFA (1 mL) and shaken at about 30° C. and shaken for about 16 h. The solvents were removed under reduced pressure. The residue in each vial was taken up in 2:1 (v/v) methanol/conc. NH₄OH (1 mL) and shaken at about 30° C. and shaken for about 16 h. The solvents were removed under reduced pressure and the residues were purified by preparative HPLC or chiral SFC to provide the title compounds in the table below, where observed LC-MS m/z is shown as [M+H]⁺.

| Ex | Structure and Name (Acid fragment) | [M + H]⁺ |
|---|---|---|
| 17 | 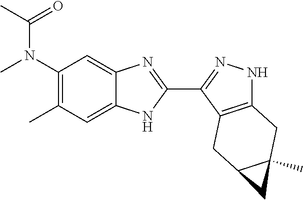 | 350 |
| | N-Methyl-N-(6-methyl-2-((4aS,5aR)-5a-methyl-1,4,4a,5,5a,6- | |
| | hexahydrocyclopropa[f]indazol-3-yl)-1H-benzo[d]imidazol-5- | |
| | yl)acetamide (acetic acid CAS: 64-19-7) | |
| 18 | 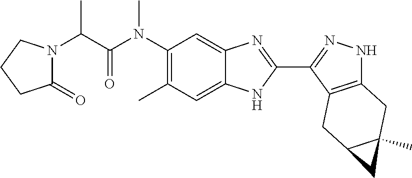 [SMILES: Cc1cc2[nH]c(-c3n[nH]c4c3C[C@@H]3C[C@]3(C)C4)nc2cc1N(C)C(=O)C(C)N1CCCC1=O] | 447 |
| | N-Methyl-N-(5-methyl-2-((4aS,5aR)-5a-methyl-1,4,4a,5,5a,6- | |
| | hexahydrocyclopropa[f]indazol-3-yl)-1H-benzo[d]imidazol-6-yl)-2-(2- | |
| | oxopyrrolidin-1-yl)propenamide | |
| | (2-(2-oxopyrrolidin-1-yl)propanoic acid CAS: 67118-32-5) | |
| 19 | 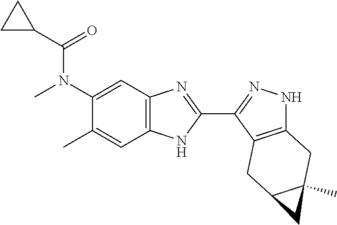 [SMILES: Cc1cc2[nH]c(-c3n[nH]c4c3C[C@@H]3C[C@]3(C)C4)nc2cc1N(C)C(=O)C1CC1] | 376 |
| | N-Methyl-N-(5-methyl-2-((4aS,5aR)-5a-methyl-1,4,4a,5,5a,6- | |
| | hexahydrocyclopropa[f]indazol-3-yl)-1H-benzo[d]imidazol-6- | |
| | yl)cyclopropanecarboxamide | |
| | (cyclopropanecarboxylic acid CAS: 1759-53-1) | |
| 20 | 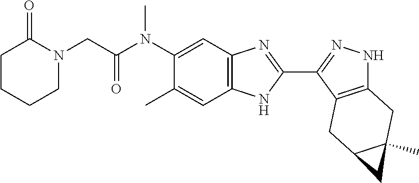 [SMILES: Cc1cc2[nH]c(-c3n[nH]c4c3C[C@@H]3C[C@]3(C)C4)nc2cc1N(C)C(=O)CN1CCCCC1=O] | 447 |
| | N-Methyl-N-(5-methyl-2-((4aS,5aR)-5a-methyl-1,4,4a,5,5a,6- | |
| | hexahydrocyclopropa[f]indazol-3-yl)-1H-benzo[d]imidazol-6-yl)-2-(2- | |
| | oxopiperidin-1-yl)acetamide | |
| | (2-(2-oxopiperidin-1-yl)acetic acid CAS: 72253-28-2) | |
| 21 | 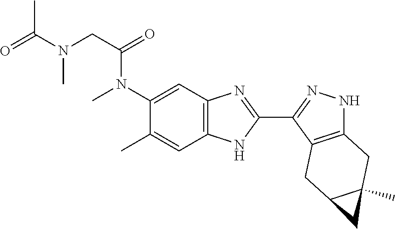 [SMILES: CC(=O)N(C)CC(=O)N(C)c1cc2nc(-c3n[nH]c4c3C[C@@H]3C[C@]3(C)C4)[nH]c2cc1C] | 421 |
| | N-Methyl-N-(5-methyl-2-((4aS,5aR)-5a-methyl-1,4,4a,5,5a,6- | |
| | hexahydrocyclopropa[f]indazol-3-yl)-1H-benzo[d]imidazol-6-yl)-2-(N- | |
| | methylacetamido)acetamide | |
| | (N-acetyl-N-methylglycine CAS: 5888-91-5) | |
| 22 | 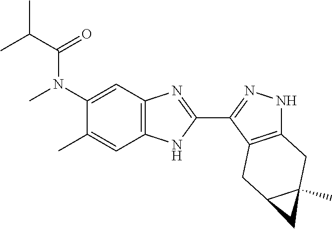 [SMILES: Cc1cc2[nH]c(-c3n[nH]c4c3C[C@@H]3C[C@]3(C)C4)nc2cc1N(C)C(=O)C(C)C] | 378 |
| | N-Methyl-N-(5-methyl-2-((4aS,5aR)-5a-methyl-1,4,4a,5,5a,6- | |
| | hexahydrocyclopropa[f]indazol-3-yl)-1H-benzo[d]imidazol-6- | |
| | yl)isobutyramide (isobutyric acid CAS: 79-31-2) | |
| 23 | 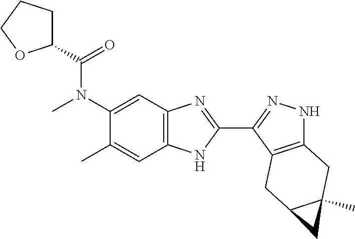 [SMILES: Cc1cc2[nH]c(-c3n[nH]c4c3C[C@@H]3C[C@]3(C)C4)nc2cc1N(C)C(=O)[C@H]1CCCO1] | 406 |
| | (R)-N-Methyl-N-(5-methyl-2-((4aS,5aR)-5a-methyl-1,4,4a,5,5a,6- | |
| | hexahydrocyclopropa[f]indazol-3-yl)-1H-benzo[d]imidazol-6- | |
| | yl)tetrahydrofuran-2-carboxamide | |
| | ((R)-tetrahydrofuran-2-carboxylic acid CAS: 87392-05-0) | |
| 24 | 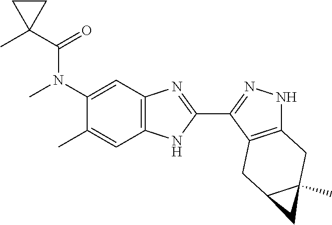 | 390 |
| | N,1-Dimethyl-N-(5-methyl-2-((4aS,5aR)-5a-methyl-1,4,4a,5,5a,6- | |
| | hexahydrocyclopropa[f]indazol-3-yl)-1H-benzo[d]imidazol-6- | |
| | yl)cyclopropane-1-carboxamide | |
| | (1-methylcyclopropane-1-carboxylic acid CAS: 6914-76-7) | |
| 25 | 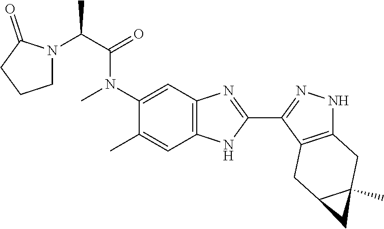 [SMILES: Cc1cc2[nH]c(-c3n[nH]c4c3C[C@@H]3C[C@]3(C)C4)nc2cc1N(C)C(=O)[C@H](C)N1CCCC1=O] | 447 |
| | (S)-N-Methyl-N-(6-methyl-2-((4aS,5aR)-5a-methyl-1,4,4a,5,5a,6- | |
| | hexahydrocyclopropa[f]indazol-3-yl)-1H-benzo[d]imidazol-5-yl)-2-(2- | |
| | oxopyrrolidin-1-yl)propenamide | |
| | ((S)-2-(2-oxopyrrolidin-1-yl)propanoic acid CAS: 96219-55-5) | |
| 26 | 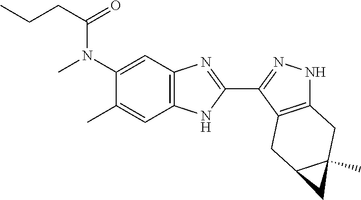 [SMILES: CCCC(=O)N(C)c1cc2nc(-c3n[nH]c4c3C[C@@H]3C[C@]3(C)C4)[nH]c2cc1C] | 378 |
| | N-Methyl-N-(6-methyl-2-((4aS,5aR)-5a-methyl-1,4,4a,5,5a,6- | |
| | hexahydrocyclopropa[f]indazol-3-yl)-1H-benzo[d]imidazol-5- | |
| | yl)butyramide (Butyric acid CAS: 107-92-6) | |
| 27 | 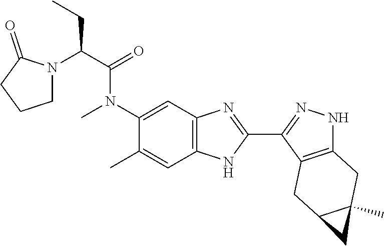 [SMILES: CC[C@@H](C(=O)N(C)c1cc2nc(-c3n[nH]c4c3C[C@@H]3C[C@]3(C)C4)[nH]c2cc1C)N1CCCC1=O] | 461 |
| | (S)-N-Methyl-N-(6-methyl-2-((4aS,5aR)-5a-methyl-1,4,4a,5,5a,6- | |
| | hexahydrocyclopropa[f]indazol-3-yl)-1H-benzo[d]imidazol-5-yl)-2-(2- | |
| | oxopyrrolidin-1-yl)butanamide | |
| | ((S)-2-(2-oxopyrrolidin-1-yl)butanoic acid CAS: 102849-49-0) | |
| 28 | 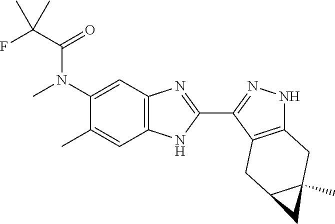 [SMILES: Cc1cc2[nH]c(-c3n[nH]c4c3C[C@@H]3C[C@]3(C)C4)nc2cc1N(C)C(=O)C(C)(C)F] | 396 |
| | 2-Fluoro-N,2-dimethyl-N-(6-methyl-2-((4aS,5aR)-5a-methyl- | |
| | 1,4,4a,5,5a,6-hexahydrocyclopropa[f]indazol-3-yl)-1H-benzo[d]imidazol- | |
| | 5-yl)propenamide | |
| | (2-fluoro-2-methylpropanoic acid CAS: 63812-15-7) | |
| 29 | 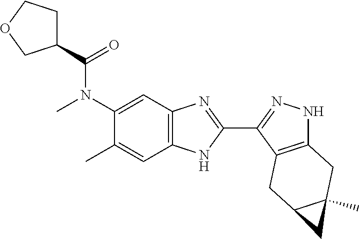 [SMILES: Cc1cc2[nH]c(-c3n[nH]c4c3C[C@@H]3C[C@]3(C)C4)nc2cc1N(C)C(=O)[C@@H]1CCOC1] | 406 |
| | (R)-N-Methyl-N-(6-methyl-2-((4aS,5aR)-5a-methyl-1,4,4a,5,5a,6- | |
| | hexahydrocyclopropa[f]indazol-3-yl)-1H-benzo[d]imidazol-5- | |
| | yl)tetrahydrofuran-3-carboxamide | |
| | ((R)-tetrahydrofuran-3-carboxylic acid CAS: 66838-42-4) | |
| 30 | 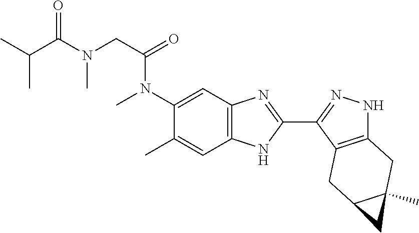 [SMILES: Cc1cc2[nH]c(-c3n[nH]c4c3C[C@@H]3C[C@]3(C)C4)nc2cc1N(C)C(=O)CN(C)C(=O)C(C)C] | 449 |
| | N-Methyl-N-(2-(methyl(6-methyl-2-((4aS,5aR)-5a-methyl-1,4,4a,5,5a,6- | |
| | hexahydrocyclopropa[f]indazol-3-yl)-1H-benzo[d]imidazol-5-yl)amino)- | |
| | 2-oxoethyl)isobutyramide | |
| | (N-isobutyryl-N-methylglycine CAS: 155256-79-4) | |
| 31 | 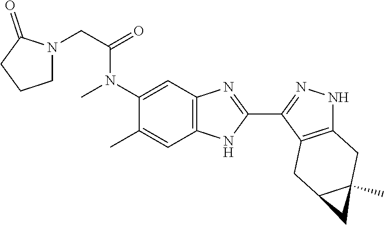 [SMILES: Cc1cc2[nH]c(-c3n[nH]c4c3C[C@@H]3C[C@]3(C)C4)nc2cc1N(C)C(=O)CN1CCCC1=O] | 433 |
| | N-Methyl-N-(6-methyl-2-((4aS,5aR)-5a-methyl-1,4,4a,5,5a,6- | |
| | hexahydrocyclopropa[f]indazol-3-yl)-1H-benzo[d]imidazol-5-yl)-2-(2- | |
| | oxopyrrolidin-1-yl)acetamide | |
| | (2-(2-oxopyrrolidin-1-yl)acetic acid CAS: 53934-76-2) | |
| 32 | 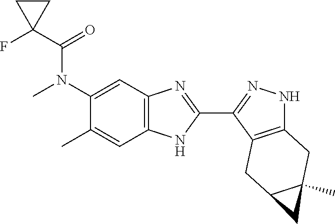 [SMILES: Cc1cc2[nH]c(-c3n[nH]c4c3C[C@@H]3C[C@]3(C)C4)nc2cc1N(C)C(=O)C1(F)CC1] | 394 |
| | 1-Fluoro-N-methyl-N-(6-methyl-2-((4aS,5aR)-5a-methyl-1,4,4a,5,5a,6- | |
| | hexahydrocyclopropa[f]indazol-3-yl)-1H-benzo[d]imidazol-5- | |
| | yl)cyclopropane-1-carboxamide | |
| | (1-fluorocyclopropane-1-carboxylic acid CAS: 137081-41-5) | |
| 33 | 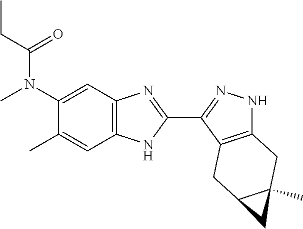 | 364 |
| | N-Methyl-N-(6-methyl-2-((4aS,5aR)-5a-methyl-1,4,4a,5,5a,6- | |
| | hexahydrocyclopropa[flindazol-3-yl)-1H-benzo[d]imidazol-5- | |
| | yl)propionamide (propionic acid CAS: 79-09-4) | |
| 34 | 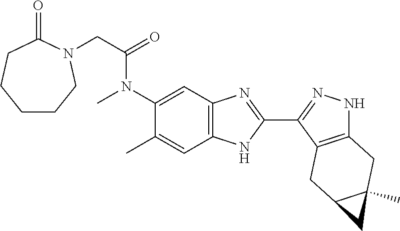 [SMILES: Cc1cc2[nH]c(-c3n[nH]c4c3C[C@@H]3C[C@]3(C)C4)nc2cc1N(C)C(=O)CN1CCCCCC1=O] | 461 |
| | N-Methyl-N-(6-methyl-2-((4aS,5aR)-5a-methyl-1,4,4a,5,5a,6- | |
| | hexahydrocyclopropa[f]indazol-3-yl)-1H-benzo[d]imidazol-5-yl)-2-(2- | |
| | oxoazepan-1-yl)acetamide | |
| | (2-(2-oxoazepan-1-yl)acetic acid CAS: 35048-56-7) | |
| 35 | 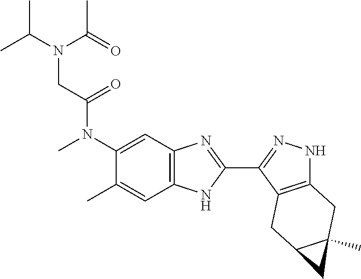 [SMILES: CC(=O)N(CC(=O)N(C)c1cc2nc(-c3n[nH]c4c3C[C@@H]3C[C@]3(C)C4)[nH]c2cc1C)C(C)C] | 449 |
| | 2-(N-Isopropylacetamido)-N-methyl-N-(6-methyl-2-((4aS,5aR)-5a- | |
| | methyl-1,4,4a,5,5a,6-hexahydrocyclopropa[f]indazol-3-yl)-1H- | |
| | benzo[d]imidazol-5-yl)acetamide | |
| | (N-acetyl-N-isopropylglycine CAS: 105260-25-1) | |
| 36 | 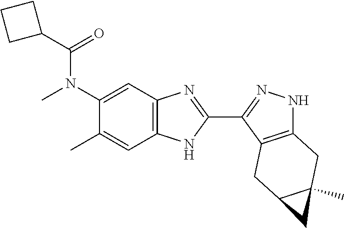 [SMILES: Cc1cc2[nH]c(-c3n[nH]c4c3C[C@@H]3C[C@]3(C)C4)nc2cc1N(C)C(=O)C1CCC1] | 390 |
| | N-Methyl-N-(6-methyl-2-((4aS,5aR)-5a-methyl-1,4,4a,5,5a,6- | |
| | hexahydrocyclopropa[f]indazol-3-yl)-1H-benzo[d]imidazol-5- | |
| | yl)cyclobutanecarboxamide | |
| | (cyclobutanecarboxylic acid CAS: 3721-95-7) | |
| 37 | 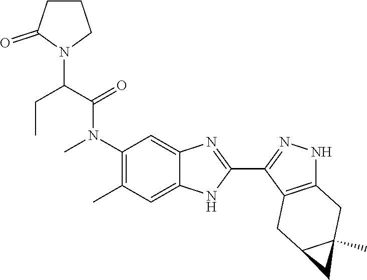 [SMILES: CCC(C(=O)N(C)c1cc2nc(-c3n[nH]c4c3C[C@@H]3C[C@]3(C)C4)[nH]c2cc1C)N1CCCC1=O] | 461 |
| | N-Methyl-N-(6-methyl-2-((4aS,5aR)-5a-methyl-1,4,4a,5,5a,6- | |
| | hexahydrocyclopropa[f]indazol-3-yl)-1H-benzo[d]imidazol-5-yl)-2-(2- | |
| | oxopyrrolidin-1-yl)butanamide | |
| | (2-(2-oxopyrrolidin-1-yl)butanoic acid CAS: 67118-31-4) | |
| 38 | 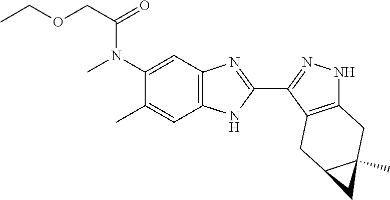 [SMILES: CCOCC(=O)N(C)c1cc2nc(-c3n[nH]c4c3C[C@@H]3C[C@]3(C)C4)[nH]c2cc1C] | 394 |
| | 2-Ethoxy-N-methyl-N-(6-methyl-2-((4aS,5aR)-5a-methyl-1,4,4a,5,5a,6- | |
| | hexahydrocyclopropa[f]indazol-3-yl)-1H-benzo[d]imidazol-5- | |
| | yl)acetamide (2-ethoxyacetic acid CAS: 627-03-2) | |
| 39 | 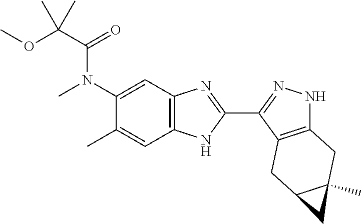 | 408 |
| | 2-Methoxy-N,2-dimethyl-N-(6-methyl-2-((4aS,5aR)-5a-methyl- | |
| | 1,4,4a,5,5a,6-hexahydrocyclopropa[f]indazol-3-yl)-1H-benzo[d]imidazol- | |
| | 5-yl)propenamide | |
| | (2-methoxy-2-methylpropanoic acid CAS: 13836-62-9) | |
| 40 | 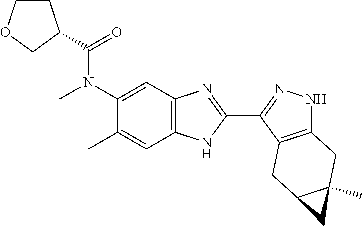 [SMILES: Cc1cc2[nH]c(-c3n[nH]c4c3C[C@@H]3C[C@]3(C)C4)nc2cc1N(C)C(=O)[C@H]1CCOC1] | 406 |
| | (S)-N-Methyl-N-(6-methyl-2-((4aS,5aR)-5a-methyl-1,4,4a,5,5a,6- | |
| | hexahydrocyclopropa[f]indazol-3-yl)-1H-benzo[d]imidazol-5- | |
| | yl)tetrahydrofuran-3-carboxamide | |
| | ((S)-tetrahydrofuran-3-carboxylic acid CAS: 168395-26-4) | |

### Biological Assays

In Vitro Studies

IL-2-Inducible T-Cell Kinase (ITK) Activity, IC₅₀ (uM)

ITK activity was determined by measuring the effect of a test compound in an ITK enzyme assay.

1.0 M HEPES Buffer pH 7.5 solution was prepared as follows: 238.3 g HEPES free acid (Sigma) and 800 mL of water were combined, and the mixture was stirred until complete dissolution. The pH was adjusted to 7.5 via titration with 5N NaOH and the volume adjusted to 1000 mL. The solution was filtered and sterilized.

ITK assay buffer was prepared as follows: 50 mL of HPLC-grade water was treated with 2 mL of 1.0 M HEPES Buffer, 500 μL of 2% Gelatin (Sigma), 1.0 mL of aqueous MgCl₂ solution (1.0 M), and 1.0 mL of aqueous glutathione solution (0.5 M), and the solution was mixed. The solution was brought to 99 mL in a graduated cylinder by addition of water and sterilized through a 0.2 μm filter. 0.1 mL of Brij-35™ Surfact-AmpS™ Detergent Solution (10% w/v aqueous solution, ThermoFisher) and 1.0 mL of ATP (Teknova, 100 mM) were added and the solution was mixed.

Preparation of 1.33×ITK enzyme solution was as follows: 49.99 mL of ITK assay buffer was treated with 4.1 μL of ITK enzyme (ITK FL (N-Flag and C-His tagged, ˜72 kDa) Lake Pharma, 0.25 mg/ml in a buffer containing 25 mM Tris pH 7.8, 150 mM NaCl, 10% glycerol and 2 mM TCEP) and the mixture was gently agitated. The resulting solution was stored on ice. 30 Minutes prior to use, the enzyme solution was removed from ice and equilibrated to RT by incubation in a RT water bath.

Preparation of 4×ITK substrate solution was as follows: 50 mL of ITK assay buffer was treated with 100 μL of BTK peptide (China Peptide Company, 2 mM stock solution in DMSO). The tube was capped, mixed by gently inverting the tube, and then stored on ice. 30 Minutes prior to use, the substrate solution was removed from ice and equilibrated to RT by incubation in a RT water bath.

At the time of assay, 7.5 μL of the 1.33×ITK enzyme solution was added to plate wells containing 0.1 μL of varying concentrations of test compound in DMSO. The plate was incubated 30 min at RT. The plate wells were each treated with 2.5 uL of the 4×ITK substrate solution and the plate was sealed (TopSeal™, Perkin Elmer). The plate was spun at 1000 rpm for 30 sec and then incubated for 60 min at RT. The seal was removed, and each well was treated with 10 μL of Stop/Detect Buffer (20 mM HEPES pH 7.5, 0.01% gelatin, 1 nM LANCE PT66 (Perkin Elmer), 16.5 μg/ml Surelight APC (Perkin Elmer), 10 mM EDTA, 250 mM NaCl). The plate was again covered and was spun at 1000 rpm for 30 seconds. The plate was allowed to incubate overnight at RT and in a closed carrier to reduce dehydration. The seal was removed, and the fluorescence was read with a plate reader with an excitation wavelength of 665 nm and an emission wavelength of 615 nm. The concentrations and resulting effect values for the tested compound were plotted and the concentration of compound required for 50% effect (IC₅₀) was determined with the four-parameter logistic dose response equation.

IC₅₀ (uM) values for compounds of the invention are presented in the Table that follows.

IL-2-Inhibition Activity, IC₅₀ (uM)

IL-2 inhibition activity in supernatants from activated CD4+ human T-cells was determined by measuring the effect of a test compound on the activity using the cisbio HTRF™ technology.

Human CD4+ T cells were activated with CD3/CD28 for 3 days and expanded for an additional 4-6 days (7 to 9 days total). On day 0, frozen CD4+ T cells were thawed, treated with CD3/CD28 Dynabeads, and incubated at 37° C./5% CO₂. On day 3, the beads were removed, and the cells were diluted to 5×10⁵ cells/cm², placed in G-Rex10 flask, and incubated at 37° C./5% CO₂. On day 7 to day 9 the cells were removed from the G-Rex flasks, counted and diluted back to 1×10⁶ cells/ml in standard tissue culture flask.

The expanded CD4+ T-cells were centrifuged at 300×g for 10 minutes and resuspended to 0.5 million cells per ml (30,000 cells/well). 60 μl of CD4+ T cells were added per well to a 384 well plate containing 0.1 μL of varying concentrations of test compound in DMSO. The plates were incubated for 15 min at 37° C./5% CO₂. 20 μl of diluted ImmunoCult™ (STEMCELL Technologies, 1:12.5 in T cell assay media) were added to all wells of the plate (1:50 final assay concentration). The plates were incubated for an additional 20 to 24 hrs at 37° C./5% CO₂. The plates were centrifuged at 300×g for 10 minutes. 16 μL of supernatant was removed and combined with 4 μl of IL-2 HTRF Abs. (cisbio kit). Plates were incubated for 3 hours at RT and read with an EnVision plate reader at 665 nm and 615 nm wavelengths. The concentrations and resulting effect values for the tested compound were plotted and the concentration of compound required for 50% effect (IC50) was determined with the four-parameter logistic dose response equation.

IC₅₀ (uM) values for compounds of the invention are presented in the Table that follows.

Tropomyosin Receptor Kinase a (TRKA) Activity, % Inhibition

Assays to determine TRKA activity are known in the art; e.g. see those described in:
Skerratt S E, et al. J. Med. Chem. (2016), 59(22):10084-10099 PMID: 27766865. DOI: 10.1021/acs.imedchem.6b00850
Bagal S K, et al., J. Med. Chem. (2018), 61(15):6779-6800 PMID: 29944371. DOI: 10.1021/acs.imedchem.8b00633

TRKA, also known as neurotrophic tyrosine kinase receptor type 1 (NTKR1) activity was determined by measuring the effect of a test compound on the activity against the NTRK1 enzyme using the ThermoFisher Z′-LYTE Assay fluorescence-based coupled enzyme format (www.thermofisher.com/selectscreen). Test compounds were screened at a fixed concentration of 1 uM and the % inhibition was determined compared to controls at a fixed ATP concentration of 1 mM. The resulting effect value for the tested compound was compared to the assay controls to determine the % inhibition (%). % Inhibition (%) values for compounds of the invention are presented in the Table that follows.

TABLE
In Vitro Study Data

| | ITK | ITK | IL-2 | IL-2 | TRKA % | TRKA |
|---|---|---|---|---|---|---|
| | IC₅₀ | count | IC₅₀ | count | inhibition | count |
| Ex # | (uM)¹ | (n) | (uM)² | (n) | (%)³ | (n) |
| 1 | 0.003 | 3 | 0.059 | 3 | NT | |
| 2 | 0.004 | 2 | 0.015 | 1 | 95 | 2 |
| 3 | 0.821 | 2 | 0.848 | 1 | NT | |
| 4 | 0.071 | 3 | 0.391 | 4 | 105 | 2 |
| 5 | 0.016 | 3 | 0.086 | 3 | NT | |
| 6 | 0.037 | 3 | 0.173 | 3 | 103 | 2 |
| 7 | 0.013 | 3 | 0.595 | 3 | 105 | 2 |
| 8 | 0.020 | 3 | 0.129 | 2 | NT | |
| 9 | 0.020 | 3 | 0.139 | 2 | NT | |
| 10 | 0.049 | 4 | 0.342 | 3 | NT | |
| 11 | 0.031 | 4 | 0.324 | 3 | NT | |
| 12 | 0.007 | 3 | 0.047 | 3 | NT | |
| 13 | 0.007 | 3 | 0.049 | 3 | NT | |
| 14 | 0.047 | 3 | 0.196 | 3 | NT | |
| 15 | 0.042 | 3 | 0.166 | 3 | NT | |
| 16 | 0.023 | 3 | 0.118 | 3 | NT | |
| 17 | 0.010 | 4 | 0.081 | 4 | 100 | 4 |
| 18 | 0.054 | 2 | 0.370 | 1 | NT | |
| 19 | 0.008 | 3 | 0.067 | 3 | NT | |
| 20 | 0.004 | 2 | 0.059 | 3 | NT | |
| 21 | 0.004 | 3 | 0.079 | 3 | NT | |
| 22 | 0.005 | 3 | 0.043 | 3 | NT | |
| 23 | 0.010 | 2 | 0.089 | 3 | 97 | 2 |
| 24 | 0.037 | 3 | 0.259 | 2 | NT | |
| 25 | 0.072 | 3 | 0.572 | 1 | NT | |
| 26 | 0.006 | 2 | 0.047 | 3 | NT | |
| 27 | 0.077 | 3 | 0.489 | 1 | NT | |
| 28 | 0.007 | 3 | 0.057 | 3 | NT | |
| 29 | 0.006 | 3 | 0.061 | 3 | 97 | 2 |
| 30 | 0.003 | 3 | 0.051 | 3 | 98 | 2 |
| 31 | 0.003 | 3 | 0.069 | 3 | 98 | 4 |
| 32 | 0.008 | 2 | 0.118 | 2 | NT | |
| 33 | 0.005 | 2 | 0.053 | 3 | NT | |
| 34 | 0.004 | 3 | 0.043 | 3 | NT | |
| 35 | 0.005 | 2 | 0.062 | 3 | 97 | 2 |
| 36 | 0.005 | 2 | 0.057 | 3 | NT | |
| 37 | 0.061 | 3 | 0.506 | 1 | NT | |
| 38 | 0.007 | 3 | 0.074 | 3 | 97 | 2 |
| 39 | 0.074 | 3 | 0.268 | 2 | NT | |
| 40 | 0.013 | 3 | 0.114 | 3 | NT | |

Key:
¹ITK IC₅₀ values are presented as a geometric mean of count n
²IL-2 IC₅₀ values are presented as a geometric mean of count n
³TRKA % inhibition values are presented as an arithmetic mean of count n
NT means not tested

1. A compound of formula (I) 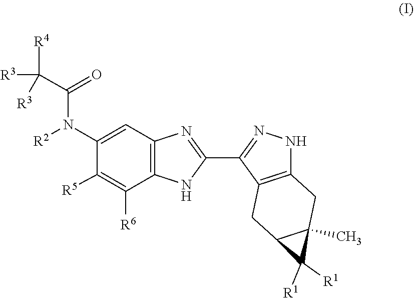
or a pharmaceutically acceptable salt thereof, wherein:
each R¹ is independently H or F;
R² is H or (C₁-C₄)alkyl;
each R³ is independently H, F or (C₁-C₄)alkyl; or
both R³ taken together with the carbon atom to which they are attached form:
a (C₃-C₆)cycloalkyl, optionally substituted by one or two F;
a (C₆-C₇)bicycloalkyl, optionally substituted by one or two F or;
a C-linked 4-7 membered saturated heterocycle containing one O;
R⁴ is H, F, (C₁-C₆)alkyl, (C₁-C₆)alkoxy, —NR⁷R⁸, or N-linked 4-8 membered saturated heterocycle containing one N and optionally one O, wherein R⁴ is not morpholinyl, wherein said heterocycle is optionally substituted by oxo;
R⁵ is H, halogen, (C₁-C₆)alkyl, (C₁-C₆)alkoxy, or (C₁-C₆)alkoxy substituted by (C₁-C₄)alkoxy;
R⁶ is independently H or F;
R⁷ is (C₁-C₆)alkyl; and
R⁸ is (C₁-C₆)alkyl or —C(O)(C₁-C₆)alkyl.

2. The compound or a pharmaceutically acceptable salt thereof according to claim 1, wherein each R¹ is H.

3. The compound or a pharmaceutically acceptable salt thereof according to claim 1, wherein R² is methyl.

4. The compound or a pharmaceutically acceptable salt thereof according to claim 1, wherein each R³ is independently H, methyl or ethyl.

5. The compound or a pharmaceutically acceptable salt thereof according to claim 1, wherein both R³ taken together with the carbon atom to which they are attached form:
a (C₃-C₆)cycloalkyl, optionally substituted by one or two F;
a (C₆-C₇)bicycloalkyl, optionally substituted by one or two F; or
a C-linked 4-7 membered saturated heterocycle containing one O.

6. The compound or a pharmaceutically acceptable salt thereof according to claim 1, wherein R⁴ is H, F, (C₁-C₃)alkyl, (C₁-C₃)alkoxy or —NR⁷R⁸.

7. The compound or a pharmaceutically acceptable salt thereof according to claim 1, wherein R⁴ is N-linked 5-7 membered saturated heterocycle containing one N and optionally one O (with the proviso that R⁴ is not morpholinyl) wherein said heterocycle is optionally substituted by oxo.

8. The compound or a pharmaceutically acceptable salt thereof according to claim 1, wherein R⁵ is H, methyl or ethyl.

9. The compound or a pharmaceutically acceptable salt thereof according to claim 1, wherein R⁶ is H.

10. A pharmaceutical composition comprising a compound of formula (I) 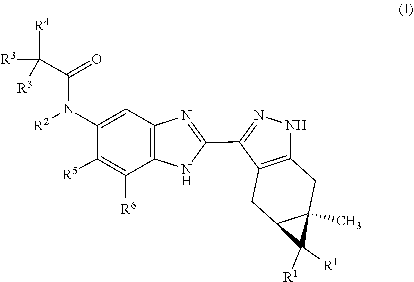
or a pharmaceutically acceptable salt thereof, wherein:
each R¹ is independently H or F;
R² is H or (C₁-C₄)alkyl;
each R³ is independently H, F or (C₁-C₄)alkyl; or
both R³ taken together with the carbon atom to which they are attached form:
a (C₃-C₆)cycloalkyl, optionally substituted by one or two F;
a (C₆-C₇)bicycloalkyl, optionally substituted by one or two F or;
a C-linked 4-7 membered saturated heterocycle containing one O;
R⁴ is H, F, (C₁-C₆)alkyl, (C₁-C₆)alkoxy, —NR⁷R⁸, or N-linked 4-8 membered saturated heterocycle containing one N and optionally one O, wherein R⁴ is not morpholinyl, wherein said heterocycle is optionally substituted by oxo;
R⁵ is H, halogen, (C₁-C₆)alkyl, (C₁-C₆)alkoxy, or (C₁-C₆)alkoxy substituted by (C₁-C₄)alkoxy;
R⁶ is independently H or F;
R⁷ is (C₁-C₆)alkyl; and
R⁸ is (C₁-C₆)alkyl or —C(O)(C₁-C₆)alkyl,
and a pharmaceutically acceptable excipient.

11. The pharmaceutical composition according to claim 10, further comprising an additional therapeutic agents.

12-15. (canceled)

16. A method of treating a disorder in a human or animal for which an ITK inhibitor is indicated, comprising administering to said human or animal a therapeutically effective amount of a compound of formula (I) 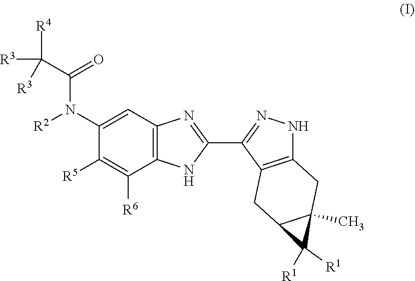
or a Pharmaceutically acceptable salt thereof, wherein:
each R¹ is independently H or F;
R² is H or (C₁-C₄)alkyl;
each R³ is independently H, F or (C₁-C₄)alkyl; or
both R³ taken together with the carbon atom to which they are attached form:
a (C₃-C₆)cycloalkyl, optionally substituted by one or two F;
a (C₆-C₇)bicycloalkyl, optionally substituted by one or two F or;
a C-linked 4-7 membered saturated heterocycle containing one O;
R⁴ is H, F, (C₁-C₆)alkyl, (C₁-C₆)alkoxy, —NR⁷R⁸, or N-linked 4-8 membered saturated heterocycle containing one N and optionally one O, wherein R⁴ is not morpholinyl, wherein said heterocycle is optionally substituted by oxo;
R⁵ is H, halogen, (C₁-C₆)alkyl, (C₁-C₆)alkoxy, or (C₁-C₆)alkoxy substituted by (C₁-C₄)alkoxy;
R⁶ is independently H or F;
R⁷ is (C₁-C₆)alkyl; and
R⁸ is (C₁-C₆)alkyl or —C(O)(C₁-C₆)alkyl.

17. The method of claim 16, wherein the disorder is a skin disorder.

18. The method of claim 17, wherein the skin disorder is dermatitis.

19. The method of claim 17, wherein the skin disorder is atopic dermatitis.

20. The method of claim 16, wherein the administering is topical.

21. The method of claim 16, wherein the compound is formulated as a cream.

22. The method of claim 16, wherein the compound is formulated as an ointment.

23. The method of claim 16, wherein the administering is oral.

24. The method of claim 16, wherein the therapeutically effective amount is from about 100 mg to about 1.5 g.
